(19) United States
(12) Patent Application Publication
(10) Pub. No.: US 2023/0226016 A1
(43) Pub. Date: Jul. 20, 2023

(54) AGENTS AND METHODS FOR TREATING TAUOPATHIES

(71) Applicant: The Regents of University of California, Oakland, CA (US)

(72) Inventors: David S. Eisenberg, Los Angeles, CA (US); Paul M. Seidler, Los Angeles, CA (US); Patrick G. Harran, Santa Monica, CA (US); Darsheed Mustafa, Los Angeles, CA (US); Melinda Balbirnie, Los Angeles, CA (US); Anton EI Khoury, Sherman Oaks, CA (US); Kevin A. Murray, Los Angeles, CA (US)

(21) Appl. No.: 17/927,639

(22) PCT Filed: May 14, 2021
(86) PCT No.: PCT/US21/32553

§ 371 Date: Nov. 23, 2022

Related U.S. Application Data
(60) Provisional application No. 63/032,124, filed on May 29, 2020.

(51) Int. Cl. A61K 31/353; A61K 31/4192; A61K 47/69; A61K 47/60; A61P 25/28

(52) U.S. Cl. CPC A61K 31/353; A61K 31/4192; A61K 47/6929; A61K 47/6923; A61K 47/60; A61P 25/28

(57) ABSTRACT

Disclosed are agents that include a flavanol (e.g., epigallocatechin-3-gallate) or a flavanol analog, a linker coupled to the flavanol or the flavanol analog, and a carrier (e.g., iron oxide nanoparticle) coupled to the linker. The disclosed agents can be used in methods for destabilizing a tau amyloid fibril, and for treating a tauopathy (e.g., Alzheimer's disease, progressive supranuclear palsy) in a subject.

### RELATED APPLICATION

This application claims a right of priority to and the benefit of the filing date of U.S. Provisional Application No. 63/032,124, filed on May 29, 2020, which is hereby incorporated by reference in its entirety.

### STATEMENT OF RIGHTS

This invention was made with government support under Grant Numbers AG029430, AG054022 and NS095661, awarded by National Institutes of Health. The government has certain rights in the invention.

### BACKGROUND

Alzheimer's disease (AD) is one among dozens of neurological disorders that are driven by aggregation of functional protein monomers into pathological fibrillar aggregates called “amyloid.” Owing to their extreme stability and self-reproducing nature, amyloids persist in brains once formed, multiply, and spread from cell to cell, killing neurons and thereby causing dementia or movement disorders.

Owing to frustration from inability to effectively target amyloids with small molecules, pharmaceutical companies have almost entirely shifted drug discovery efforts from small molecule programs to the development of biologics, such as antibodies, for targeted inhibition of amyloids. The limitation of biologics, especially antibodies, is that they have little ability to penetrate the brain and cell membranes to reach cell interiors, the location of important amyloid targets. As an additional problem, antibodies are extremely expensive to produce, which limits their potential impact on the general population, since billions of aging people are expected to require anti-amyloid therapies. As a result, it is unlikely that antibody production will meet the desired criteria as a robust and economical anti-amyloid treatment.

No structures of any inhibitors bound to any amyloids have been described. Whereas compounds with in vitro anti-amyloid activity are known, none are proven clinical therapeutics, often lacking in vivo efficacy for reasons including limited bioavailability, poor metabolic stability, poor drug-like properties, and lack of specificity. Therefore, there is a need in the field for improved agents for treating amyloid diseases, especially tauopathies.

### SUMMARY OF THE INVENTION

The disclosed embodiments are based, at least in part, on the determination of a cryo-EM structure of EGCG bound to brain-extracted fibrils of tau from an Alzheimer's patient, which reveals that columns of EGCG wedge into a crevice at the interface between the two tau protofilaments of pathogenic paired helical filaments, forcing the protofilaments apart and weakening the electrostatic forces that bind the layers of tau molecules into the fibril. Achieving this structure required careful timing of electron microscope grid preparation after addition of EGCG to the brain-derived fibrils, permitting the complex to form but not to disaggregate the fibrils. One significance of the structure is that it reveals a pharmacophore-like binding site on pathogenic tau and suggests the mechanism of action of EGCG's ability to disaggregate toxic amyloid.

In some aspects, the disclosed agents comprise a flavanol or a flavanol analog, a linker coupled to the flavanol or the flavanol analog, and a carrier coupled to the linker.

In some embodiments, the flavanol or the flavanol analog comprises a compound selected from Table J. In some embodiments, the linker comprises a compound selected from Table L. In some embodiments, the carrier comprises a nanoparticle. In some embodiments, the nanoparticle has a hydrodynamic particle size that is at least 4 nanometers and at most 150 nanometers as measured by dynamic light scattering. In some embodiments, the carrier comprises an iron oxide nanoparticle (IONP) (e.g., selected from Table N). In certain embodiments, the carrier comprises a coating that comprises polyethylene glycol (PEG), dextran, starch, chitosan, lipid, citrate, polyaniline, meso-2,3-dimercaptosuccinic acid, poly(maleic anhydride-alt-1-octadecene), polyacrylamide, phosphonate, or silica. In certain embodiments, the coating comprises dextran, e.g., wherein the dextran comprises dextran-20 kDa, dextran-40 kDa, carboxy dextran, and/or cross-linked dextran-20 kDa. In some embodiments, the carrier is coupled to the linker via said PEG.

In some embodiments, the agent further comprises an anti-amyloid antibody or peptide coupled to the carrier for the purposes of aiding the delivery of the agent to the target of interest, and/or interfering with the aggregation of the said amyloid by capping the target fibril as an additional mode of amyloid inhibition. In certain embodiments, the agent destabilizes a tau amyloid fibril when in contact with said tau amyloid fibril. In some embodiments, the agent is permeable across the blood-brain barrier.

In certain aspects, the disclosed compositions comprise one or more of the agents described above and in the rest of the disclosure. Such compositions, in some embodiments, are effective in treating Alzheimer's disease.

In certain aspects, the disclosed methods of preparing one or more of the agents described above and in the rest of the disclosure comprise reacting EGCG with a bromine-activated molecule to couple the linker and EGCG. In some embodiments, such methods further comprise reacting the molecule with succinic anhydride or N-hydroxysuccinimide to form an activated linker. In certain embodiments, these methods further comprise reacting the activated linker with the carrier to form the agent.

In certain aspects, the disclosed methods of preparing one or more of the agents described herein comprise reacting the flavanol or the flavanol analog with propargyl bromide, thereby forming a propargylated compound. In some embodiments, such methods further comprise reacting the propargylated compound with an amino azide and tris((1-benzyl-4-triazolyl)methyl)amine, thereby forming a conjugate of the flavanol or the flavanol analog with a linker. In certain embodiments, these methods further comprise reacting the conjugate with the carrier, thereby covalently attaching the two to each other, to form the agent.

In some aspects, the disclosed methods of treating a tauopathy in a subject comprise administering to the subject an effective amount of a composition that comprises one or more of the agents described above and in the rest of the disclosure.

In some embodiments, the compounds and compositions described herein are administered intravenously. In some embodiments, the tauopathy comprises Alzheimer's disease. In other embodiments, the tauopathy comprises progressive supranuclear palsy, which is shown also to be sensitive to treatment with EGCG, or chronic traumatic encephalopathy, which is structurally-related to AD-tau fibrils that contain the EGCG binding cavity that is described here.

In certain aspects, the disclosed methods of destabilizing a tan amyloid fibril comprise contacting the tau amyloid fibril with one or more of the agents described above and in the rest of the disclosure.

In some embodiments, the tau amyloid fibril comprises a paired helical filament. In certain embodiments, the agent dis-aggregates the paired helical filament, e.g., by disrupting ion pairing of lysine at position 340 of tau (i.e., position 340 of SEQ ID NO: 1) by forming at least one hydrogen bond with said lysine.

Further embodiments and details for each of these aspects are presented throughout the disclosure.

### BRIEF DESCRIPTION OF THE FIGURES

FIG. 1 shows inhibition of seeding by known small molecule amyloid inhibitors measured in tau-K18 biosensor cells using tau fibrils from AD brain tissue as seeds. (a) Seeding following overnight preincubation of AD brain purified tau fibrils with a final inhibitor concentration of 10 μM. Seeding was measured 3 days after transfecting biosensor cells with fibril-inhibitor complexes. A dashed magenta line marks 50% inhibition. Error bars show the standard deviation of triplicate measurements. (b-d) Representative images from seeding inhibition experiments in a. Red arrows point to representative cells containing seeded tau aggregates, and white arrows point to representative cells lacking aggregated tau. (e) EGCG dose dependence on seeding by fibrils of recombinant tau40. Seeding (expressed as 10 power) was measured as in a, and IC50 values were calculated from dose-response plots. (f) Heat maps showing inhibition of seeding by crude brain extracts from donors with AD (Left, N=15) or PSP (Right, N=7). Each column represents the inhibitor response of a different donor. Donor information is given in Table 1. Inhibitor concentrations were 10 μM. Responses to inhibitors were normalized to seeding in the absence of inhibitor, abbreviated “No Inh”, and normalized seeding values for each inhibitor were plotted from maximal inhibitor response (low fractional seeding, left) to minimal response (seeding approaching a value of 1.0, right) for each of the indicated inhibitors. The second row, labeled “Theo Perfect Inh”, shows the performance of a theoretically perfect inhibitor that reduces fractional seeding to 0. Samples that were inhibited by at least 50% by treatment with WIW or lacmoid are indicated with text and a white arrow. All patient samples were inhibited by at least 50% by treatment with EGCG.

FIG. 2 shows that EGCG binds to and disaggregates tau fibrils. (a) Isothermal titration calorimetry (ITC) of 750 μM EGCG into 75 μM recombinant tau-K18+ fibrils. The binding isotherm was fit using a standard 1 site binding model. (b) ThT assay showing the effect EGCG or negative control, doxycycline, have on the aggregation of 20 μM tau-K18+ monomer³³, a construct that contains all of the residues that are observed in the AD tau fibril core^{7,9}. Note the sudden drop in ThT signal at 10 hrs (marked by a red arrow) for the sample containing EGCG. (c) Negative stain electron micrographs of 75 μM recombinant tau-K18+ fibrils prior to incubation with EGCG (top) or following 1 or 3 hour incubation with a two-fold molar excess of EGCG at room temperature, as indicated. (d) As in c except EGCG micrographs of tau are of fibrils purified from AD brain tissue and EGCG treatments were carried out at a concentration of 100 μM for a 3 hour or overnight duration at 37° C., as indicated.

FIG. 3 shows the CryoEM structure of AD brain-derived tau fibril bound to inhibitor, EGCG. (a) Cross-section of fibril from 3D classification showing strand separation, with a spacing of 4.8 Δ. (b) Published map of AD-brain derived tau PHF (PDB 5O3L), viewed down the fibril axis, for comparison with c. (c) Cryo-EM density map of AD-brain derived tau PHF complexed with EGCG. Yellow arrows in c indicate new densities that are attributed to bound EGCG, and dashed triangles in b show the position of the inhibitor binding cleft mapped to the unliganded fibril. See text for explanation of numbering. (d) Overlay of EGCG-bound (green) and unliganded (black, PDB 5O3L) tau PHFs showing 2.3 Å separation of tau protofilaments in the EGCG-bound form and imposed clash of EGCG with H329 in the unliganded conformation. EGCG is shown as yellow/red space fill and is marked with a magenta wedge. Note the overlap of His 329 from unliganded structure with the EGCG ligand. H-bonds are marked with gray dashed lines. (e) EGCG ligand (yellow) modeled into 3.9 Å resolution map.

FIG. 4 shows that the homopeptide recreates the EGCG binding domain. (a) The tau PHF (rendered as gray spacefill) is formed by stacks of identical protofilaments, labeled Protofilaments 1 and 2. Stacked protofilaments are colored in alternating light and dark gray to aid discernment. EGCG (yellow) binds in a cleft that is created by the dimerization of a 14-residue homopeptide segment of the tau protofilament, referred to as PHFE2 (boxed region colored in green and highlighted with transparent black lines). (b) Fibrils formed by the PHFE2 peptide. Inset shows AD-brain derived tau PHFs for comparison. Red lines highlight similar crossover distances of fibrils of PHFE2 peptide and AD brain-derived tau PHFs. (c) ThT signal of fibrils formed from PHFE2 peptide without EGCG, or following 30 min incubation. Error bars show the standard deviation of triplicate measurements.

FIG. 5 shows the wedge-charge-destabilization hypothesis for fibril disaggregation by EGCG. (a) Overlay of EGCG-bound (green) and unliganded (black, PDB 6HRE) tau PHFs showing separation of tau protofilaments in the EGCG-bound form, and imposed clash of EGCG with Asn327 and His329 in the unliganded conformation. EGCG is shown as yellow/red space fill. Inset shows the depicted region of the PHF, and the anticipated wedging effect of EGCG, which forces the protomer interface apart in counteracting directions from binding in the two symmetry-related clefts. (b) Alternating negatively (red) and positively (blue) charged sidechains of the p-helix segment of AD-tau shown from the enlargement of the boxed region of the Inset, viewed down the fibril axis. Stabilization of the fibril structure is provided by pairing of neighboring oppositely charged residues (pairs shown by boomerang-shaped brackets). (c) By forming a H-bond with Lys340 of tau (dotted black line), bound EGCG diminishes the effect of ion pair-mediated charge stabilization with neighboring negative glutamates, Glu338 and Glu342, thereby increasing inter-layer repulsion of unpaired glutamates in neighboring layers. This repulsion is expected to weaken inter-layer bonding, favoring fibril disruption.

FIG. 6 shows the Fourier Shell Correlation (FSC) plot used to determine resolution of 432-pixel data set using a 0.5 FSC cutoff, as indicated with orange lines. FSC analysis was performed using postprocess in Relion⁴², and confirms lack of overfitting owing to strong agreement between the unmasked (green), masked (blue) and corrected black) curves. In addition, the curve plotted for the phase randomized masked map (red) shows sharply approaches zero indicating lack of correlation of noise between two half maps, also suggesting maps lack indication of overfitting.

FIG. 7 shows how EGCG contacts with the Paired Helical Filament polymorph of AD brain-derived tau. Multiple binding modes of the EGCG inhibitor in the binding cavity of the tau PHF can be modeled, as shown. Model 1 is judged to be of best qualitative fit as it satisfies the greatest number of favorable protein contacts with: His329. Asn327 and Lys340 (of the opposite protein chain). Model 2 is a rotation of the EGCG pose in Model 1 (relative to the fibril axis), and also makes favorable contacts with His329 and Asn327. Model 1 is distinguished from Model 2 in that the EGCG pose in Model 2 projects a H-bond donor, the hydroxyl of the phenyl ring, towards Lys340, reducing the chance of H-bonding. Model 3 a rotation of Models 1 and 2, fails to fit the density as well as either Model 1 or 2, exhibits a clash of the phenyl ring of EGCG with His329.

FIG. 8 shows the EGCG sites anticipated to be compatible with AD-tau binding and nanoparticle/linker conjugation. Protein-ligand contacts and shielding obstruct sites in red from solvent, and hence are not expected to retain AD-tau binding when modified to carry linkers for nanoparticle conjugation. Sites shown in green are solvent-facing and hence are to be compatible with AD-tau binding when modified to carry linkers for nanoparticle conjugation. The sites shown in green are, with respect to the orientation and nomenclature shown in FIG. 14, the two on the A ring the two on the D ring (excluding the one on D ring closest-as-drawn to the C ring, which, along with the three on the C ring, is shown in red in the original drawing).

FIG. 9 shows the assigned conjugation site in the context of a conjugation reaction.

FIG. 10 shows how the seeded aggregates are affected by the tested compounds. Images from seeding inhibition experiments are also shown, specifically for AD-tau+EGCG (control).

FIG. 11 shows how the seeded aggregates is affected by the tested compounds. Images from seeding inhibition experiments are also shown, specifically for AD-tau+CNS-set 7220009.

FIG. 12 shows examples of improved lead compounds, including CNS-set 7953084.

FIG. 13 shows the rationale for covalent conjugation of EGCG to nanoparticles: Reduced binding to off-target globular proteins with retained binding to fibrils of AD-tau (A) and AD-tau Fibril with exposed columns of bound EGCG (B). Nanoparticles-fusion as a means of reducing EGCG off-target binding (A) EGCG binding sites in off-target globular proteins are buried deep inside of solvent-excluded pockets (marked by red arrows and yellow-filled circles), which are inaccessible to nanoparticle fused EGCG due to steric constraints. Metabolic enzymes share similar properties with model globular proteins shown in A, having buried active sites with restricted binding to nanoparticle-fused EGCG. (B) EGCG binding sites in AD-tau fibril determined from cryoEM structure (Seidler P M et al., bioRxiv 2020). EGCG (yellow) binds in two solvent-accessible column-shaped clefts marked by red columns with yellow fill. EGCG binding sites remain accessible to nanoparticle-fused EGCG given its surface-exposed character. Thus, EGCG binding site in fibrils of AD-Tau are uniquely solvent-exposed and retain binding to nanoparticle-fused EGCG, and nanoparticle fusion offers a route to improving drug-like properties of EGCG by inhibiting EGCG binding to off-target proteins and metabolic enzymes.

FIG. 14 shows a CryoEM structure of non-liganded AD-tau fibrils (A, PDB 6HRE) and fibrils bound to disaggregant EGCG (B), from reference Seidler bioRxiv 2020). EGCG is rendered green with oxygens shown in red. Residues from the Tau protein are rendered grey with oxygens red, nitrogens blue and sulfur gold. The surface on EGCG that remains solvent accessible in the fibril-bound pose is labeled. (C) Chemical structure of EGCG showing the nomenclature of ring systems. Density map of EGCG-tau binding cleft (green—EGCG, blue/grey—tau fibril).

FIG. 15 shows Scheme 1 of Example 5 for properly assigning the structure of monopropargylated EGCG.

FIG. 16 shows the reaction for Table 3 of Example 5 for protecting group free, copper catalyzed Huisgen cycloaddition to form aminoPEGylated EGCG^{a}.

FIG. 17 shows the reaction for Table 4 of Example 5 for a set of EGCG derivatives having varied linker lengths^{a}.

FIG. 18 shows Scheme 2 of Example 5 for rapid and selective A-ring deuteration of EGCG conjugates.

FIG. 19 shows that linker conjugated EGCG analogs retain inhibitory activity towards AD crude brain extracts. (A) Seeding by crude AD brain extract pre-treated with EGCG or experimental linker-conjugated analogs, as indicated. Inhibitor activity is read-out by measuring seeding in tau biosensor cells. Seeding is taken as a proxy for the fibril load that is contained within the AD crude brain extracts. Reduction in fibril load following treatment with experimental linker-conjugated analogs of EGCG reduces prion-like seeding by AD-tau nearly as effectively as EGCG itself. (B) Representative fluorescence images of tau biosensor cells experiments from A. Intracellular aggregates seeded by crude AD brain extracts are identified as puncta (green dots in the “No inhibitor” treated sample, left fluorescence micrograph). Inhibitor treatment reduces the number of puncta (right fluorescence micrograph). The number of puncta as a function of inhibitor pre-treatment is plotted in A.

FIG. 20 shows that nanoparticle-conjugated EGCG retains inhibitor activity and clusters with fibrils of AD-tau. (A) Seeding by crude AD brain extract measured in tau biosensor cells that were co-transfected with nanoparticles coupled to EGCG by linkers of varying length. (B-C) Negative-stain electron micrographs of EGCG-conjugated and non-conjugated nanoparticles. Nanoparticle coupled with EGCG analog 5c (B) cluster with fibrils of AD-tau. No clustering is seen between non-conjugated nanoparticles and AD-tau fibrils (C).

### DETAILED DESCRIPTION OF THE INVENTION

The disclosed methods relate, at least in part, to structure-based design of a novel class of drugs for Alzheimer's Disease and other tauopathies.

In the context of Alzheimer's disease, amyloid aggregates of tau are recognized as a cause of the disease. If tau amyloid could be disaggregated, there are strong reasons to believe that the progression of Alzheimer's disease could be halted. Some challenges to developing such a drug are first, to get the drug across the blood-brain-barrier and into neurons where tan aggregates residue, and then to break down the tan aggregates.

The structure-based design disclosed herein achieves both steps by coupling a natural product amyloid disaggregator, EGCG, via a synthetic linker to iron-oxide nanoparticles that cross the blood-brain-barrier and enter cells. EGCG is a flavonoid in green tea known to somehow disaggregate amyloid, but it has poor drug-like properties and has failed efficacy in clinical trials. The designed construct, alternatively referred to as an agent in this disclosure, links in some embodiments a modified EGCG (or a flavanol or a flavanol analog) with improved in vivo stability to iron oxide nanoparticles that cross the blood-brain-barrier. This design has been enabled by a cryo-electron microscopic determination of the structure of EGCG bound to tau amyloid fibrils. This structure reveals how EGCG binds to and weakens the non-covalent bonds holding tau molecules into the fibril. From the structure one can deduce the moiety of EGCG that is available for linkage to the nanoparticle. The disclosed designs include the linker itself, the linkage site on EGCG (or a flavanol or a flavanol analog), and the nanoparticle.

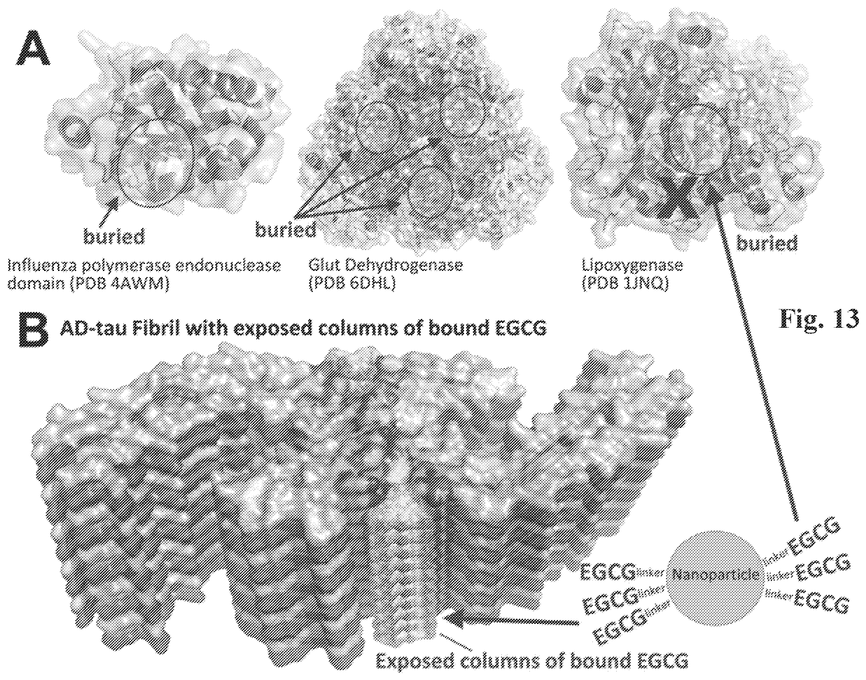

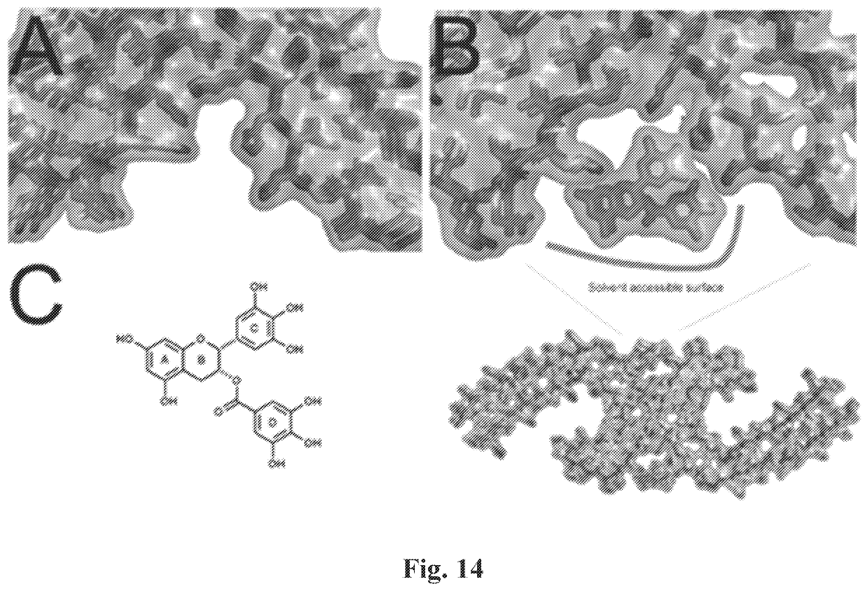

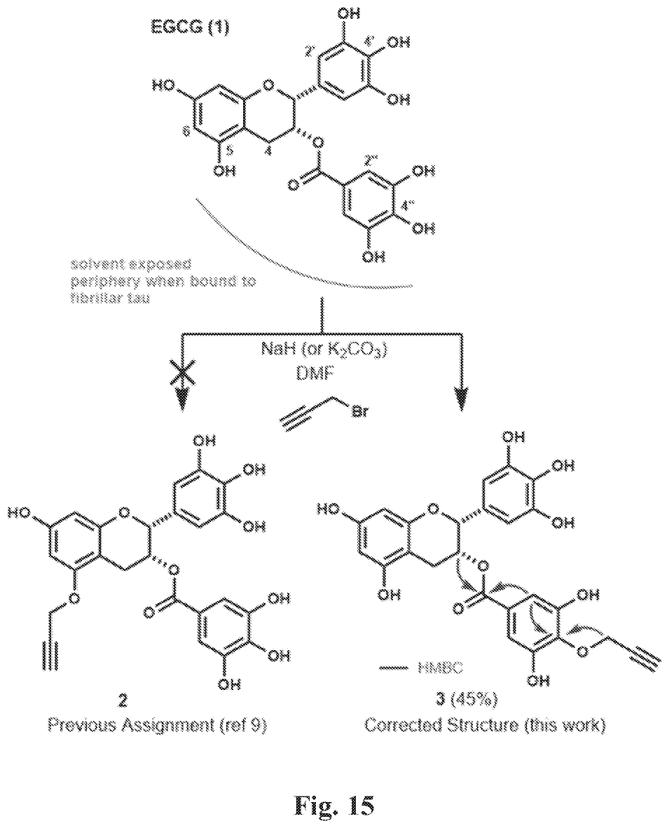

EGCG lacks optimal properties for convenient therapeutic use, and has been limited by low brain permeability, poor metabolic stability and significant off-target protein binding. Based on the structure of EGCG bound to fibrils of AD-tau (Seidler P M et al., bioRxiv 2020), we disclose here the design and synthesis of linker-conjugated analogs of EGCG that, when coupled with nanoparticles that are known to cross the blood-brain-barrier and enter cells, retain inhibitory activity towards Alzheimer's tau. Our disclosed EGCG-linker analog conjugates offer a means to improving the drug-like properties of EGCG by reducing binding to metabolic and other off-target globular proteins (FIG. 13).

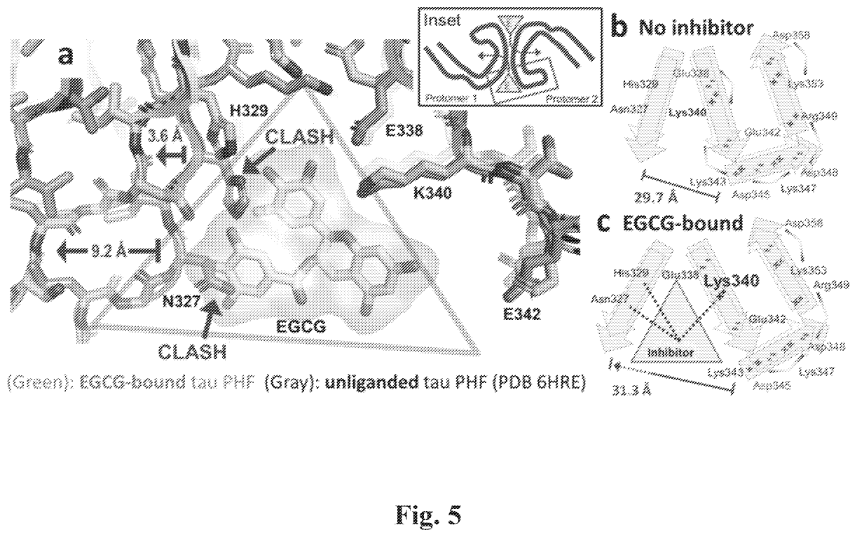

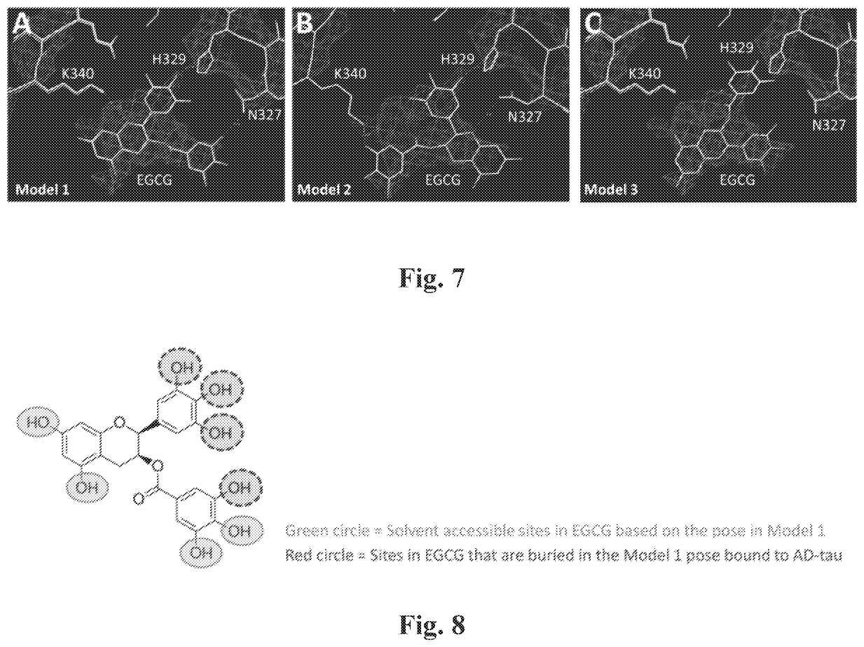

Our data show that the D-ring of EGCG is amenable to site-specific derivatization for linker conjugation with and subsequent conjugation with the nanoparticle carrier Ferumoxytol. Our design was informed by the cryo-electron microscopic structure of EGCG bound to AD-tau amyloid fibrils (FIG. 5, FIG. 7, and FIG. 8). From these structures, we deduced that the D-ring of EGCG is compatible with linker/nanoparticle conjugation. We also find that the D-ring of EGCG is most susceptible to site-specific conjugation with synthetic linkers.

We describe here methodology to couple EGCG (or a flavanol or a flavanol analog) with nanoparticles to create an amyloid-specific and metabolically stabilized entity owing to nanoparticle fusion. The EGCG binding sites in globular proteins that account for off-target binding are well studied and reside inside of deep, solvent-excluded pockets that bear no resemblance to the AD-Tau EGCG binding cleft (FIG. 13). To the contrary, EGCG binds to a solvent-accessible surface of AD-tau fibrils in a column-shaped cleft that runs contiguously along the fibril. We exploit this observation by fusing EGCG to nanoparticles to overcome off-target binding by sterically interfering with the internalization of EGCG into buried binding pockets of globular proteins. Thus, nanoparticle fusion offers a rational path to reducing off-target binding of EGCG without precluding binding to AD-Tau.

Our approach is to make covalent EGCG-nanoparticle conjugates that exhibit functional binding to AD-tau and restricted binding to off-target globular proteins by virtue of steric clashes between the fused nanoparticle carrier and buried active sites in off-target globular proteins.

The disclosure, among other things, describes the mechanism of amyloid fibril reversibility that is exerted by the natural product EGCG, as discovered by the disclosed structural study of tau amyloid fibrils that were purified from the brain of an AD patient, complexed with EGCG. From this structure, rules were distilled to guide the discovery and design of small molecules that reverse amyloid fibrils of tau from AD by inducing (a) electrostatic repulsions between layered protein sheets of the AD tau fibril, and (b) physical destabilization of the AD tau fibril by targeting a cryptic inhibitor binding site that resides in the most common tau fibril polymorph in AD patients (see Examples 1 through 6).

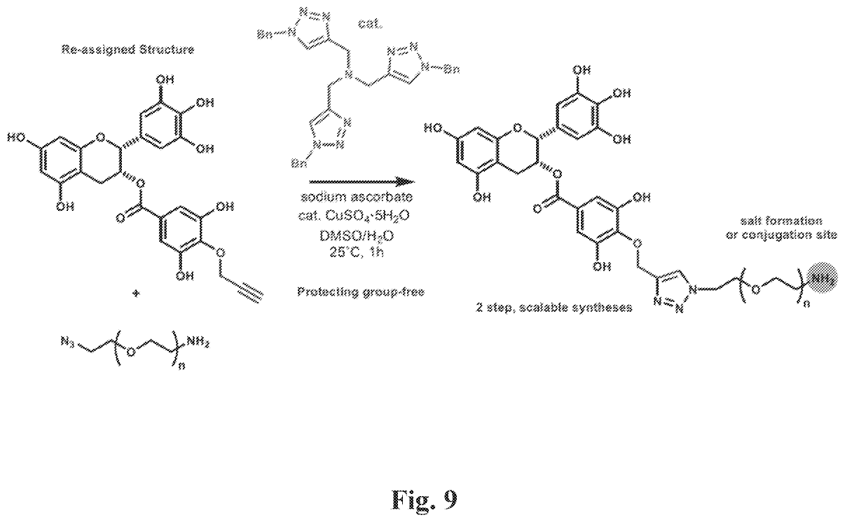

Based on these data, this disclosure (1) delineates moieties of EGCG that are either (a) important for target engagement with AD brain-derived tau fibrils, or (b) dispensable, and hence subjectable to derivatization (FIG. 8); (2) describes a single site on EGCG that is amenable to site-specific derivatization for conjugation of EGCG to nanoparticle carriers, and/or other excipients (such as PEGylation), which can be used to enhance target specificity, cell penetration, brain penetration, and biostability (FIG. 9); and (3) provides the pharmacophore model of the AD brain-derived tau fibril (FIG. 5), which allows alternative scaffolds to be envisaged for the rational de novo design of small molecules and biologics that inhibit the tau PHF, or other amyloids with similar structural architecture.

The disclosed designed constructs (i.e., agents) are amenable to inexpensive production. EGCG is a non-toxic natural product. Iron oxide nanoparticles have been approved by the FDA for several applications, and are non-toxic.

### Definitions

As used in the description, the words “a” and “an” can mean one or more than one. As used in the claims in conjunction with the word “comprising,” the words “a” and “an” can mean one or more than one. As used in the description, “another” can mean at least a second or more.

The term “treating” includes curing, relieving, or ameliorating to any extent a symptom of an illness or medical condition or preventing further worsening of such a symptom. For example, treating Alzheimer's disease includes making the Alzheimer's disease less severe.

The phrase “pharmaceutically-acceptable carrier” as used herein means a pharmaceutically-acceptable material, composition or vehicle, such as a liquid or solid filler, diluent, excipient, solvent or encapsulating material. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin: (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol: (12) esters, such as ethyl oleate and ethyl laurate: (13) agar; (14) buffering agents, such as magnesium hydroxide and aluminum hydroxide; (15) alginic acid: (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “subject” refers to a mammal, including, but not limited to, a human or non-human mammal, such as a bovine, equine, canine, ovine, or feline.

### Agents and Compositions

In some aspects, the present disclosure provides agents that are effective in disaggregating a tau amyloid fibril or that can be used to treat a tauopathy. The agents, in various embodiments, can destabilize a tau amyloid fibril when in contact with the tau amyloid fibril. In some embodiments, the agents are permeable across the blood-brain barrier.

Such disclosed agents include a flavanol or a flavanol analog, a linker coupled to the flavanol or the flavanol analog, and a carrier coupled to the linker. The flavanol or the flavanol analog can be epigallocatechin-3-gallate (EGCG), a structure of which is depicted in FIG. 8. FIG. 8 shows that, based on the present disclosure, four of the hydroxyl groups (circled in green) are amenable to conjugation. Therefore, the linker can be attached to EGCG at those positions. FIG. 9 shows one potential scheme for attaching a linker to EGCG. Further details of the scheme are provided in Example 5. One of skill in the art will recognize that the elements of the conjugate can be coupled in other orders using other coupling procedures, e.g., by reacting EGCG with activated linkers coupled to iron oxide particles, for example, to prepare the conjugates as disclosed herein.

In some embodiments, the carrier includes a nanoparticle, such as an iron oxide nanoparticle (IONP). An IONP can further be coated through suitable means, such as those described in Arami H. et al., In vivo delivery, pharmacokinetics, biodistribution and toxicity of iron oxide nanoparticles, Chem Soc Rev. 44(23): 8576-8607 (2015). As an example, the coating may include polyethylene glycol (PEG), dextran (e.g., dextran-20 kDa, dextran-40 kDa, carboxy dextran, or cross-linked dextran-20 kDa), starch, chitosan, lipid, citrate, polyaniline, meso-2,3-dimercaptosuccinic acid, poly(maleic anhydride-alt-1-octadecene), polyacrylamide, phosphonate, or silica. The carrier can be coupled to the linker via the coating, for example via PEG. In some embodiments, the agent can also include an anti-amyloid antibody coupled to the carrier.

The present disclosure also provides compositions for treating a tauopathy. The compositions, in various embodiments, include the agents described above and in the rest of this disclosure. Such compositions can be effective in treating tauopathies, such as Alzheimer's disease.

The composition may comprise a carrier (e.g., a pharmaceutically-acceptable carrier, for any of the disclosed embodiments). The pharmaceutical compositions disclosed herein may be delivered by any suitable route of administration (e.g., oral, intravenous), and can be supplied in various forms (e.g., powders, ointments, drops, liquids, gels, tablets, capsules, pills, or creams).

Below, Table J provides, with further references to other tables or parts of this disclosure, some examples of flavanols or flavanol analogs that can be a part of the described agents. Similarly, Table L provides some examples of linkers, and Table N provides some examples of iron oxide nanoparticles (an example of a carrier).

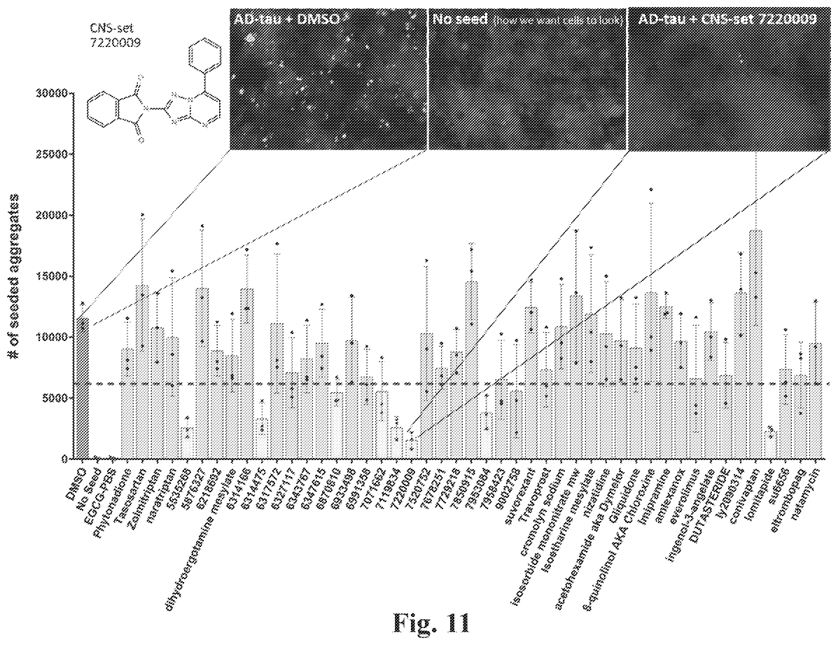

TABLE J
Examples of flavanols or flavanol analogs
Compounds listed in Table A of Example 1
Compounds listed in Table B of Example 1
Compounds 5a, 5b, 5c, 5d, 5e described in
Example 5 (which also include a conjugated
linker)
Compounds listed in the x-axis of FIG. 11
Compounds listed in Table C of Example 6
Compounds listed in Table D of Example 6
Compounds listed in Table E of Example 6
Compounds listed in Table F of Example 6
Compounds listed in Table G of Example 6

TABLE L
Examples of linkers
Compounds 4a, 4b, 4c, 4d, 4e described in
Example 5 (in pre-conjugated form)
Compounds listed in Table H of Example 6
(shown as conjugated to flavanols or flavanol
analogs)

TABLE N
Examples of iron oxide nanoparticles (a type of carrier)
Polyglucose sorbitol carboxymethylether-coated iron
oxide (ferumoxytol) (e.g., ferumoxytol AMI-7228)
Carboxydextran-coated iron oxide (ferucarbotran)
(e.g., ferucarbotran SHU555A, ferucarbotran SHU555C)
Dextran-coated iron oxide (ferumoxtran) (e.g.,
ferumoxtran-10 AMI-227)
Dextran-coated iron oxide (ferumoxide) (e.g.,
ferumoxide AMI-24)
Feruglose NC100150

### Methods of Destabilizing a Tau Amyloid Fibril and of Treating a Tauopathy

In some aspects, the present disclosure provides methods for treating a tauopathy in a subject. Such methods include administering to the subject an effective amount of a composition disclosed in the “Agents and Compositions” section as well as the rest of this disclosure. The administration of the compositions can be intravenous. The treated tauopathy, in some embodiments, includes Alzheimer?s disease and/or progressive supranuclear palsy.

The selected dosage level, as can be determined by a medical practitioner, will depend upon a variety of factors including the activity of the particular agent employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

The present disclosure also provides methods for destabilizing a tau amyloid fibril. Such methods include contacting the tau amyloid fibril with an agent disclosed in the “Agents and Compositions” section as well as the rest of this disclosure. The tau amyloid fibril may include a paired helical filament, and the used agent can dis-aggregate that paired helical filament, for example by disrupting ion pairing of lysine at position 340 of tau with respect to SEQ ID NO: 1 by forming at least one hydrogen bond with said lysine.

>SEQ ID NO: 1
MAEPRQEFEVMEDHAGTYGLGDRKDQGGYTMHQDQEGDTDAGLKESPLQ
TPTEDGSEEPGSETSDAKSTPTAEDVTAPLVDEGAPGKQAAAQPHTEIP
EGTTAEEAGIGDTPSLEDEAAGHVTQARMVSKSKDGTGSDDKKAKGADG
KTKIATPRGAAPPGQKGQANATRIPAKTPPAPKTPPSSGEPPKSGDRSG
YSSPGSPGTPGSRSRTPSLPTPPTREPKKVAVVRTPPKSPSSAKSRLQT
APVPMPDLKNVKSKIGSTENLKHQPGGGKVQIINKKLDLSNVQSKCGSK
DNIKHVPGGGSVQIVYKPVDLSKVTSKCGSLGNIHHKPGGGQVEVKSEK
LDFKDRVQSKIGSLDNITHVPGGGNKKIETHKLTFRENAKAKTDHGAEI
VYKSPVVSGDTSPRHLSNVSSTGSIDMVDSPQLATLADEVSASLAKQGL

### EXAMPLES

### Example 1: CryoEM Reveals how the Small Molecule EGCG Binds to Alzheimer's Brain-Derived Tau Fibrils and Initiates Fibril Disaggregation

EGCG, the most abundant flavanol in green tea, is one of the few natural compounds known to inhibit amyloid fibril formation of proteins associated with neurodegeneration, and to disaggregate amyloid fibrils. Little is known of the mechanism of molecular action of EGCG, or how it or other small molecules interact with amyloid fibrils. Here we present a 3.9 Å resolution cryoEM structure that reveals the site of EGCG binding to Alzheimer's disease (AD) brain-derived tau fibrils. The structure suggests that EGCG disaggregates fibrils by wedging into a cleft that forms between the two tau protofilaments, and by causing charge repulsions between tau layers of the fibril. In support of this, we observe separation of the protofilaments that EGCG wedges between, and accompanying displacement of the adjacent pi-helix domain. We find that EGCG inhibits pathogenic seeding by human brain extracts from 15 donors with AD, and 7 with progressive supranuclear palsy (PSP). In short, our structure suggests a mechanism for the disaggregation of amyloid fibrils by EGCG, and defines a pharmacophore-like cleft in the AD tau fibril.

### Introduction

Single-particle cryoEM has delivered spectacular structures of protein amyloid fibrils¹⁻⁵, including several of Alzheimer's associated tau purified from pathogenic human brain tissues⁶⁻¹⁰. These structures offer the opportunity to learn atomic-level details of how small molecules inhibit and disaggregate amyloid fibrils. Epigallocatechin gallate (EGCG), a small molecule natural product in green tea, inhibits aggregation of amyloid proteins, and disaggregates fibrils into smaller units, despite their great stability.¹¹⁻¹⁵ To illuminate the action of EGCG on tau fibrils, we determined the single-particle cryoEM structure of the AD-associated brain-derived paired-helical filament (PHF) polymorph of tau in complex with EGCG.

Aggregated tau forms fibrillar structures that propagate and cause neurodegeneration by prion-like seeding.¹⁶⁻¹⁷ Single-particle cryoEM structures of brain-purified tau from donors with AD show that pathological tau fibrils, like other amyloids, involve combinations of homo- and heteromeric steric zippers with rich p character, strong shape complementarity and tightly interdigitated sidechains^{7,9}. The structures formed by amyloid fibrils are particularly stable owing to steric zipper interactions, and an extensive network of polarized hydrogen bonds (H-bonds) that forms along the fibril axis.^{18,19} The binding enthalpies of the resulting amyloid fibril structures rival that of crystalline ice.²⁰ These features give rise to the extreme stability that is characteristic of pathogenic amyloid fibrils, which can withstand heating to boiling temperatures, and resist solubilization in detergents such as sarkosyl. The tremendous stability of pathogenic amyloid explains its ability to seed new fibrils and to evade degradation by cellular machinery; and at the same time, presents the question of how amyloid fibrils can be disaggregated.

Chaperones function as high molecular weight homo- and heteromeric assemblies that use chemical energy to disaggregate yeast prions and certain amyloid fibrils.^{21,22} Amyloid disaggregation is an attractive potential therapeutic mechanism of action, and the efficacy of chaperone-mediated disaggregation can be tuned by protein engineering to target pathological aggregates.^{23,21} Remarkably EGCG, a small molecule natural product, is also reported to inhibit at least 14 different amyloids, in part by disaggregating amyloid fibrils, and also by blocking the aggregation of protein monomers.^{11-13,15,25}

Despite its potent in vitro anti-amyloid activity. EGCG is not a proven clinical therapeutic. Its lack of clinical efficacy stems from poor drug-like properties, which include limited bioavailability and lack of specificity. In addition, EGCG is known to interact with numerous biomolecules, often with differing ligand poses²⁶⁻³⁰, and has been described as a pan-assay interfering compound.³¹ Thus it is not surprising that EGCG is reported to modulate the activity of dozens of different disease-related pathways and proteins³². Despite EGCG's limitations as a clinical agent, we sought to understand how EGCG interacts with, and inhibits the AD tau amyloid fibril polymorph. The EGCG binding site we discovered on the AD tau fibril is quite unlike EGCG binding sites in globular proteins, and constitutes a tau amyloid pharmacophore that can possibly be exploited to create and discover alternative AD small-molecule inhibitors with improved drug-like properties.

### Selection of a Small Molecule Inhibitor for Structure Determination

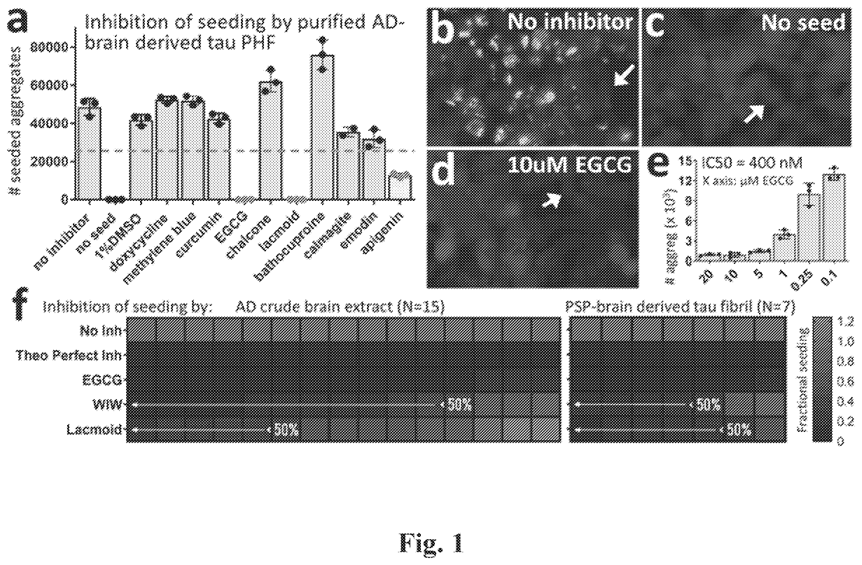

To identify small molecules that interfere with seeding by AD tau fibrils, we screened a panel of 9 compounds that are reported to bind to various amyloids. We found 3 compounds that blocked the ability of tau fibrils purified from AD brain to seed new fibrils in tau biosensor cells (FIG. 1a-d). The most effective inhibitors of seeding were lacmoid and epigallocatechin gallate (EGCG), the latter having an IC50 of 400 nM (FIG. 1e). EGCG was the most consistent inhibitor of seeding among 15 AD crude brain extracts tested (FIG. 1f), operating essentially as a “theoretically perfect inhibitor” that reduces fractional seeding by every AD brain sample to zero (Row 2 of FIG. 1f). For comparison, the structure-based inhibitor of tau called WIW, which we previously developed as an inhibitor of AD³³, blocked seeding by 80% of crude AD brain extracts from the same panel. In addition, we found that EGCG blocked seeding by crude brain extracts from donors with another tauopathy called progressive supranuclear palsy (PSP), also with near perfect efficacy. On these bases, we selected the small molecule EGCG as the focus of our structural and biochemical studies.

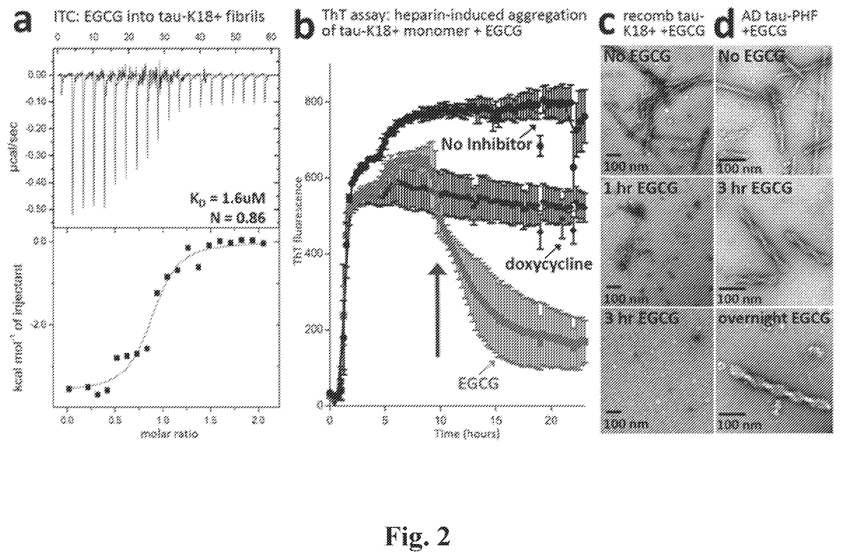

EGCG disaggregates tau by stoichiometric binding to fibrils Biochemical and electron microscope studies indicate that EGCG, the best small molecule inhibitor of seeding that we identified (FIG. 1f), binds tau fibrils with a stoichiometry of nearly one molecule of EGCG for each molecule of tau, and then breaks down the fibrils. By isothermal titration calorimetry (ITC) we found that EGCG binds recombinant tau-K18+ fibrils with an affinity of 1.6 μM and a near equimolar stoichiometry with an N of 0.86 (FIG. 2a). To examine the effect of EGCG on amyloid formation of tau-K18+, we carried out the thioflavin T assay shown in FIG. 2b.

We observed no shift in the lag time of tau aggregation, suggesting that EGCG has no inhibitory effect on the tau-K18+ monomer. However, fibrils formed in the presence of EGCG were metastable insomuch as the ThT signal abruptly fell after reaching a plateau at 10 hr (FIG. 2b), suggesting that EGCG binding either displaces the ThT dye from the fibrils, or causes tau fibrils to disaggregate. Negative staining electron microscopy shows that EGCG disaggregates the fibrils (FIG. 2c). An alternative explanation that EGCG prevents the fibrils from adhering to the EM grid is less likely because the disappearance of fibrils increases from the 1 to 3 hr time points in spite of all other variables remaining unchanged, and because others report that EGCG disaggregates fibrils of other amyloids including α-synuclein, amyloid β, and apolipoprotein A-I.^{12,15}

To prepare specimens of AD brain-derived tau fibrils for structural studies, we examined negatively stained EM grids of purified tau fibrils from the frontal cortex of AD donor 1 (FIG. 1a) at various times following incubation with EGCG. AD brain-derived tau fibrils were more robust than recombinant tau fibrils and disaggregation was accelerated by incubation at 37° C. As shown in FIG. 2d before incubation with EGCG, brain-extracted tau fibrils appear as relatively narrow twisted filaments with a high abundance on the EM grid. A 3 hr incubation with EGCG produced AD tau fibrils with radically different characteristics compared to non-inhibitor treated fibrils (compare FIG. 2d top and middle), which were broadened and more braided in appearance. Following overnight incubation with EGCG, most of the AD brain-derived tau fibrils are lost; although some remain, unlike recombinant tau fibrils, which virtually vanish after a 3 hr incubation with EGCG. The AD brain-extracted tau fibrils that remained after overnight incubation with EGCG appeared broadened and overtly shabby (FIG. 2d, bottom). Therefore, we incubated AD brain-derived fibrils with EGCG for 3 hr at 37° C. immediately prior to preparing cryoEM grids for single-particle cryoEM data collection.

### CryoEM Structure of AD Tau Fibril Bound to EGCG

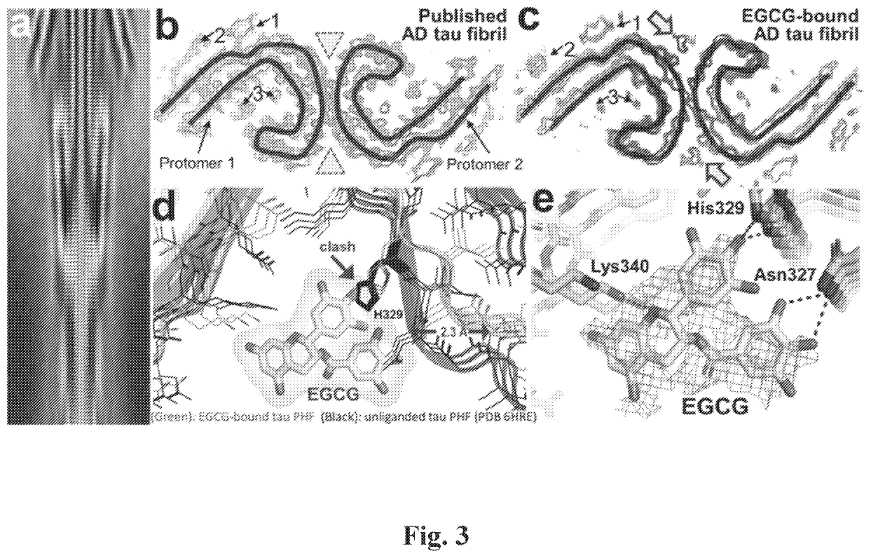

We carried out helical reconstruction of EGCG-treated tau fibrils purified from AD brain donor 1 using unbiased 3D classifications of manually picked particles with a cylindrical reference having an outer diameter of 250 pixels (266 Å). Only the tau PHF polymorph was seen in our 3D reconstructions suggesting that the other known AD tau fibril polymorph, straight filaments, were either particularly sensitive to treatment with EGCG, or too few to observe. It is noteworthy that straight filaments are a minor species in the AD brain.^{7,9} As shown in FIG. 3a, the spacing between individual β-strands was apparent in the 3D projection of aligned particles extracted using a 686-pixel box with a resolution of 4.4 Å (determined using a 0.5 FSC cutoff). From this initial low resolution map, we were able to observe density for EGCG, and also other conserved densities that have been noted in all four other published structures of AD brain-derived tau PHFs;^{7,9} specifically: (1) islands of density proposed to derive either from ubiquitination³⁴ or the N-terminal segment, 7-EFE-9, around residues K317 and K321^{9,35}, (2) a large patch of density near Q307, V309 and K311 thought to be ubiquitin³⁴ and/or steric zipper interaction with other tau sequences³³, and (3) two patches of density in the interior core of the fibril (compare FIGS. 3b and c, labels 1-3).

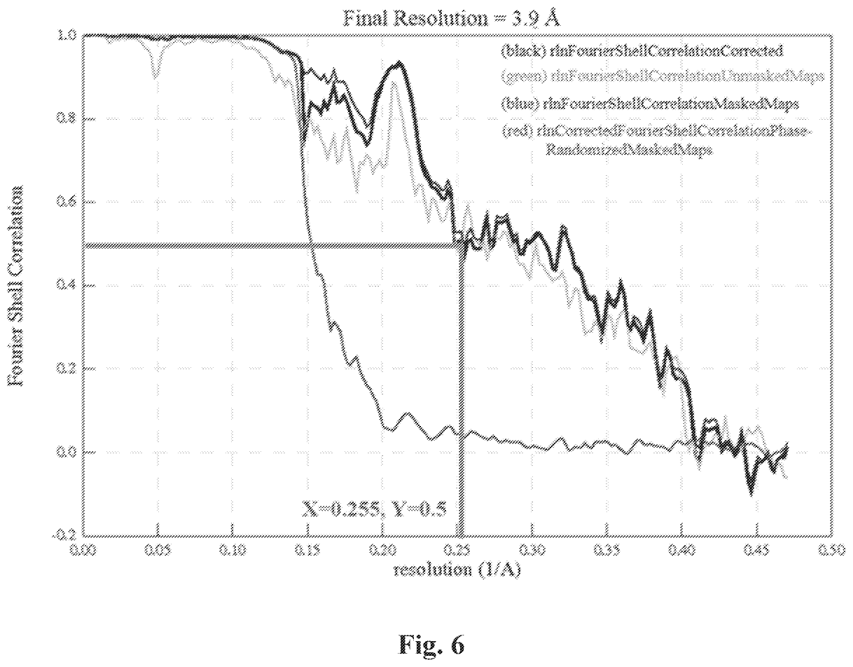

In addition to patches of conserved density, we observed two new peaks highlighted by yellow-filled arrows in FIG. 3c, which lie in a cleft that is formed by the junction of two tau protofilaments. The intensity of these symmetry related peaks, which we attribute to bound EGCG, is approximately equal to conserved density 1, described above. Owing to the site of EGCG binding in the cleft formed at the PHF interface. EGCG wedges the protofilaments of the dimer apart by ˜2.3 Å (FIG. 3d and FIG. 5c). To visualize the atomic contacts made by EGCG with the tau fibril, we obtained a higher resolution map by re-extracting the high resolution subset of particles from the 686-pixel box boxes with smaller box sizes. Attempts to re-extract with a box smaller than 432 pixels failed to yield high resolution particle alignments, however 3D classification using a 432-pixel box size produced a 3.9 Å electron density map (see FSC in FIG. 6).

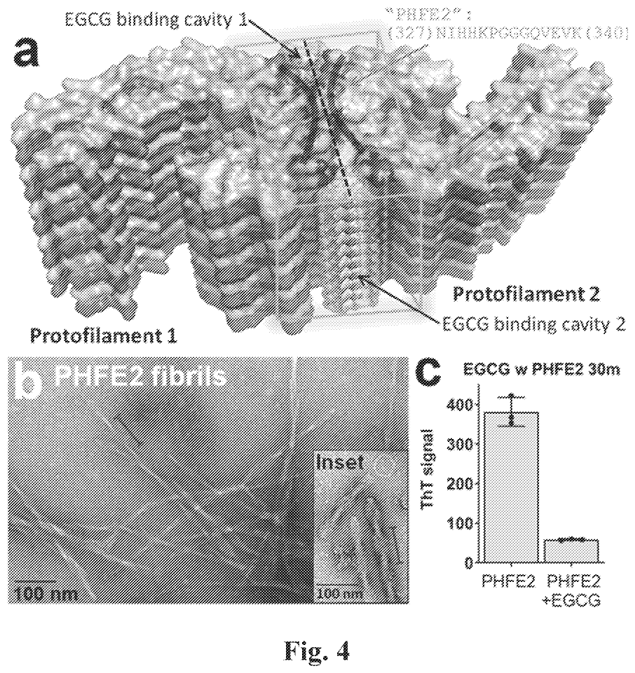

Modeling EGCG into the electron density of the 3.9 Å resolution map revealed a single inhibitor pose shown in FIG. 3e that is superior to the competing inhibitor binding poses that we considered (FIG. 7). Molecules of EGCG stack in two separate columns along the fibril axis in an equimolar ratio making equivalent contacts in each symmetry related site (FIGS. 3e and 4a). The hydroxyl of one monocyclic ring of EGCG H-bonds with His329, and a hydroxyl from the other monocyclic ring of EGCG H-bonds with Asn327 of the same protofilament (FIG. 3e). A third H-bond is made between Lys340 from the opposite tau protofilament and the oxygen of the ether in the bicyclic moiety of EGCG. In addition, each bound EGCG molecule is close enough to EGCG molecules in the layers above and below to form self-stabilizing n-stacking interactions by all three rings of the stacked ligands (FIG. 4a).

### Validation of the EGCG Binding Cleft

The model of EGCG bound to the AD brain-derived tau fibril shown in FIG. 3 suggests that the EGCG binding cleft is created by a homomeric peptide interface that is formed by two tau protofilaments involving residues 327-340 with amino acid sequence NKHHKPGGGQVEVK, referred to here as “PHFE2”. If so, we would expect that the PHFE2 peptide could pair to form amyloid-like fibrils in the absence of the other 427 residues of tau, and that the resulting fibrils should recreate the EGCG binding cleft (FIG. 4a). Accordingly, we find fibrils of PHFE2 have a crossover distance that is similar to AD brain-derived PHFs (FIG. 4b and inset showing AD brain-derived tau fibrils for comparison), although PHFE2 fibrils are much narrower than brain-derived PHFs, as expected, since the PHFE2 peptide lacks most of the residues that are present in brain-derived tau fibrils. In this respect, PHFE2 fibrils are akin to a protein domain, which we hypothesized retains EGCG binding.

We tested the ability of EGCG to disrupt fibrils of PHFE2, which harbors the putative EGCG binding cleft. The results, shown in FIG. 4c reveal that PHFE2 fibrils possess a strong ThT signal, consistent with electron micrographs demonstrating fibril formation. Addition of EGCG to the same batch of PHFE2 fibrils eliminated nearly any measurable ThT signal, suggesting that PHFE2 fibrils indeed recreate the EGCG binding cleft, and the associated inhibitor-mediated fibril reversibility.

### Identification of Alternative Disaggregator of Tau Improved Drug-Like Properties Based on the AD-Tau Pharmacophore

An alternative route to a clinically promising inhibitor is to discover surrogate brain-penetrating small molecule disaggregators of AD-tau with drug-like properties that are better than EGCG. Towards that end, we screened a carefully curated subset of small molecule by in silico docking using the AD-tau pharmacophore that is defined by the EGCG binding cavity. To increase the chances of identifying small molecule surrogates that are more amenable for clinical translation, we carried out in silico docking using compound libraries that derived from (1) a subset of compounds from LOPAC (Library of Pharmacologically Active Compounds) that were pre-filtered on the basis of having predicted brain-penetration, and (2) ChemBridge CNS-set, a library containing drug-like compounds that have increased probability of brain-penetration and oral bioavailability. Docking was carried using AutoDock for both FDA-approved compounds from LOPAC, and the CNS-set library. For the LOPAC library of FDA-approved compounds, docking was also carried out with RosettaDock, which yielded a slightly different ranking of top-scoring compounds. The 25 best-scoring compounds each docking experiment are shown below. It is possible that any of these could compounds that are discovered based on the reported AD-tau pharmacophore could serve as valuable lead compounds, given their established drug-like properties and predicted activity as disaggregators of AD-tau.

TABLE A

| | FDA-approved | FDA-approved |
|---|---|---|
| | hit compounds | hit compounds |
| Rank | (RosettaDock) | (AutoDock) |
| 1 | Docarpamine | Ergotamine |
| 2 | Gliquidone | BMS-201038, Lomitapide |
| 3 | Arteflene | Dihydroergotamine nasal |
| 4 | Naratriptan | Temoporfin |
| 5 | Fudosteine | Tasosartan |
| 6 | Lanoconazole | Candesartan |
| 7 | Nizatidine | Everolimus |
| 8 | Acetohexamide | Dihydroergocristine |
| 9 | Phytonadione | Dutasteride |
| 10 | Sarecycline | Amlexanox |
| 11 | Tubocurarine | Teniposide |
| 12 | Phentermine | Saprisartan |
| 13 | Levobupivacaine | Ingenol mebutate |
| 14 | Isoetharine | MK-8228, Letermovir |
| 15 | Prolixin decanoate | Eltrombopag |
| 16 | Sulfamethazine | Conivaptan |
| 17 | Isosorbide Mononitrate | Irbesartan |
| 18 | Travoprost | Fenquizone |
| 19 | Natamycin | Cyclothiazide |
| 20 | Phenylbutazone | Suvorexant |
| 21 | Agrimophol | Raltegravir |
| 22 | Trimetrexate | Ajmalicine |
| 23 | Sodium cromoglycate | Dihydroergotamine |
| 24 | Imipramine | Idarubicin |
| 25 | Zolmitriptan | Daunorubicin |

TABLE B

| Rank | CNS-set hit compounds (AutoDock) |
|---|---|
| 1 | 10,12-dimethyl-10,12-dihydro-7H,11H-benzo[de]imidazo[4′,5′:5,6]benzimidazo |
| | [2,1-a]isoquinoline-7,11-dione |
| 2 | 2-(7-phenyl[1,2,4]triazolo[1,5-a]pyrimidin-2-yl)-1H-isoindole-1,3(2H)-dione |
| 3 | 6-chloro-3-[5-(3-fluorophenyl)-1,3,4-oxadiazol-2-yl]-2H-chromen-2-one |
| 4 | 4-(4-fluorophenyl)-2-methyl-5-oxo-N-2-pyridinyl-1,4,5,6,7,8-hexahydro-3- |
| | quinolinecarboxamide |
| 5 | 4-(3-methoxyphenyl)-2-methyl-5-oxo-N-2-pyridinyl-1,4,5,6,7,8-hexahydro-3- |
| | quinolinecarboxamide |
| 6 | 2-methyl-4-(2-methylphenyl)-N-(6-methyl-2-pyridinyl)-5-oxo-1,4,5,6,7,8- |
| | hexahydro-3-quinolinecarboxamide |
| 7 | 9-(1,3-benzodioxol-5-yl)-3,3,6,6-tetramethyl-3,4,6,7,9,10-hexahydro-1,8(2H,5H)- |
| | acridinedione |
| 8 | 2-methyl-N-(6-methyl-2-pyridinyl)-5-oxo-4-(3-pyridinyl)-1,4,5,6,7,8-hexahydro- |
| | 3-quinolinecarboxamide |
| 9 | N-(tert-butyl)-N-(difluoromethyl)-4-methylbenzenesulfonamide |
| 10 | 6-bromo-3-(3,4-dihydro-2(1H)-isoquinolinylcarbonyl)-2H-chromen-2-one |
| 11 | N-mesityl-2-(3-oxoindeno[1,2,3-de]phthalazin-2(3H)-yl)acetamide |
| 12 | 1-(3-chlorophenyl)-3-(3,5-dimethylphenyl)tetrahydro-1H-thieno[3,4-d]imidazol- |
| | 2(3H)-one 5,5-dioxide |
| 13 | N-(4-methyl-2-pyridinyl)-2-[(5-methyl-5H-[1,2,4]triazino[5,6-b]indol-3-yl)thio] |
| | acetamide |
| 14 | 2-methyl-4-(4-methylphenyl)-N-(4-methyl-2-pyridinyl)-5-oxo-1,4,5,6,7,8- |
| | hexahydro-3-quinolinecarboxamide |
| 15 | 2-methyl-5-oxo-N-2-pyridinyl-4-(2-thienyl)-1,4,5,6,7,8-hexahydro-3- |
| | quinolinecarboxamide |
| 16 | N-(6-methyl-2-pyridinyl)-2-oxo-2H-chromene-3-carboxamide |
| 17 | N-[3-(trifluoromethyl)phenyl]-10H-phenothiazine-10-carboxamide |
| 18 | 2-phenyl-4,5,6,7-tetrahydro-8H-cyclopenta[d]pyrazolo[1,5-a]pyrimidin-8-one |
| 19 | N-[5-(3-methyl-4-oxo-3,4-dihydro-1-phthalazinyl)-2-(1-piperidinyl)phenyl] |
| | acetamide |
| 20 | 5,14-dioxo-5,14-dihydrobenzo[5,6]indolo[1,2-b]isoquinoline-13-carbonitrile |
| 21 | N-(2-methoxy-5-methylphenyl)-2-(2-oxobenzo[cd]indol-1(2H)-yl)acetamide |
| 22 | 3-(1H-benzimidazol-1-yl)-1-(2,4-dimethylphenyl)-2,5-pyrrolidinedione |
| 23 | 3-(2,3-dihydro-1,4-benzodioxin-2-yl)-6-phenyl-7H-[1,2,4]triazolo[3,4-b][1,3,4] |
| | thiadiazine |
| 24 | 2-[3-(3-methylphenyl)-1,2,4-oxadiazol-5-yl]-N-(3-pyridinylmethyl)benzamide |
| 25 | N-(2-methoxyphenyl)-3-oxo-3H-benzo[f]chromene-2-carboxamide |

### Discussion

The EGCG inhibitor binding cleft on the tau PHF is quite unlike binding sites of small molecules on globular and membrane proteins²⁶⁻³⁰. Firstly, it is not a single site, but rather is a contiguous column that is created by thousands of stacked inhibitor binding clefts. Secondly, each of these many clefts bristles with charged and polar sidechains, ready to form hydrogen bonds with some of the 11 hydrogen-bond acceptors, and 8 hydrogen-bond donors on EGCG. FIG. 5a shows the charged sidechains on the protofilament to the left of EGCG; these include Glu338, Lys340, and continuing along the p-helix Glu342, Lys343, Asp345, and Arg347. Notice that the charges of these sidechains alternate negative and positive. In fact, the pattern of alternating acids and bases makes the PHF structure possible: in order for like charges to stack roughly 4.8 Å apart along the fibril axis, neighboring residues on the same level can form ion pairs to partially neutralize charge repulsion between identical residues in adjacent layers. We define this phenomenon as intra-layer charge neutralization. Perhaps also helping to achieve charge neutralization are bound water molecules and/or posttranslational modifications such as the acetylation of Lys343³⁴. Potential hydrogen bonding residues on the other protofilament include Asn327, His329, and Lys331.

Our Wedge-Charge-Neutralization hypothesis is shown in FIGS. 5b and c. By rotation about its single bonds, EGCG adopts a roughly planar wedge to fit into the wedge-shaped clefts at the junctures of the two protofilaments of the PHF of tau. EGCG, by being in position to form hydrogen bonds with Lys340, His329, and Asn327, diminishes intra-layer charge neutralization, thereby increasing inter-layer charge repulsion of stacked charged residues. For example, by H-bonding with Lys340, EGCG disrupts ion pairing with neighboring acidic residues, and increases inter-layer repulsion by creating stacks of unpaired lysines. Lys340 is particularly susceptible to the effect of charge destabilization since it is solvent excluded by the protofilament interface, and is increasingly excluded from solvent by the column of bound EGCG molecules. The disruption of ion pairing in the solvent excluded environment of amyloid fibrils is found to be detrimental to aggregation, whereas outward pointing residues with unpaired charge appear to be somewhat more tolerated.³⁶

EGCG binding at the protofilament junction also destabilizes the tau PHF by forcing apart the two protofilaments that form the dimer interface (FIGS. 5b and c). The wedge effect of EGCG binding is apparent both from the translation of the protofilament backbone adjacent to the EGCG binding cleft, shown in FIG. 3d, and from breaks in the density at Lys340 and the hinge point of Gly355 (FIG. 5a). These are the only breaks in density that are observed in the structure of the EGCG complex, and suggest that EGCG exerts its effect primarily by perturbing the β-helix domain. In short, we attribute the dissolution of fibrils by EGCG to the combined wedge effect of forcing the protofilaments apart, and the inhibition of charge neutralization, which weakens the binding of layers to one another.

In summary, the structure of EGCG bound to AD brain-derived tau fibrils illustrates how an exceptionally rich H-bonding molecule that nestles between protofilaments of amyloid fibrils can weaken interactions that otherwise stabilize fibrils. In addition, the structure defines what might be termed a pathogenic amyloid pharmacophore. Previous suggestions of how small molecules interact with, and inhibit fibrils have been mainly limited to computational docking. The structure of the pharmacophore of the AD brain-derived tau PHF opens the door to informed screening and the design of small molecules with drug-like properties.

### Example 2: Experimental Methods for Example 1

### Recombinant Protein Expression and Purification

Human Tau tau-K18+(residues Q244-E380 of 4R tau) was expressed in a pNG2 vector in BL21-Gold E. coli cells grown in LB to an OD600=0.8. Cells were induced with 0.5 mM IPTG for 3 hours at 37° C. and lysed by sonication in 20 mM MES buffer (pH 6.8) with 1 mM EDTA, 1 mM MgCl₂, 1 mM DTT and HALT protease inhibitor before addition of NaCl 500 mM final concentration. Lysate was boiled for 20 minutes and the clarified by centrifugation at 15,000 rpm for 15 minutes and dialyzed to 20 mM MES buffer (pH 6.8) with 50 mM NaCl and 5 mM DTT. Dialyzed lysate was purified on a 5 ml HighTrap SP ion exchange column and eluted over a gradient of NaCl from 50 to 550 mM. Protein was polished on a HiLoad 16/600 Superdex 75 pg in 10 mM Tris (pH 7.6) with 100 mM NaCl and 1 mM DTT, and concentrated to ˜20-60 mg/ml by ultrafiltration using a 3 kDa cutoff.

### Preparation of Crude and Purified Brain-Derived Tau Seeds

Tissue for neuropathologically confirmed tauopathy cases from brain regions indicated in the figure legend were fresh-frozen, and extracted without freeze-thaw. A brief description of the characteristics of each donor is provided in Table 1. Tissue was cut into a 0.2-0.3 g section on a block of dry ice, and then manually homogenized in a 15 ml disposable tube in 1 ml of 50 mM Tris, pH 7.4 with 150 mM NaCl containing 1×HALT protease inhibitor. Samples were then aliquoted to PCR tubes and sonicated in a cuphorn bath for 120 min under 30% power at 4° C. in a recirculating ice water bath, according to reference³⁷. For purification of PHFs and SFs from AD brain tissue, extractions were performed according to the previously published protocol without any modifications⁹.

### CryoEM Data Collection and 3D Reconstruction

AD brain purified tau fibrils were applied to negatively glow-discharged Quantifoil 1.2/1.3 electron microscope grids (2.6 μl for 1 min), and subsequently plunge-frozen in liquid ethane on a Vitrobot Mark IV (FEI). Data were collected on a Titan Krios (FEI) microscope equipped with a Gatan Quantum LS/K2 Summit direct electron detection camera (operated with 300 kV acceleration voltage and slit width of 20 eV). Counting mode movies were collected on a Gatan K2 Summit direct electron detector with a nominal physical pixel size of 1.07 Å per pixel with a dose per frame 1.26 e-/Å2. A total of 30 frames with a frame rate of 5 Hz were taken for each movie, resulting in a final dose 38 e-/Å2 per image. Automated data collection was driven by the Leginon automation software package.³⁸

Data were processed as described by Boyer et al.¹ Briefly. CTF estimation was performed using CTFFIND 4.1.8³⁹, and Unblur⁴⁰ was used to correct beam-induced motion. Particle picking was performed manually using EMAN2 e2helixboxer.py⁴¹, and particles were extracted and classified in RELION⁴² using the 90% overlap scheme. Particles were extracted using a 686-pixel box size for 2D classifications and a first series of 3D classifications. Typically, we performed Class3D jobs with K=3 and manual control of the tau_fudge factor and healpix to reach a resolution of ˜5-6 Å to select for particles that contributed to the highest resolution class. The deposited maps and atomic coordinates derived from the subset of particles of 3D classification with a 686-pixel box size, re-extracted using a 432-pixel box. Two subsequent rounds of 3D classification were carried out for the 432-pixel box, followed by a single K=1 Class3D job. We performed the map-map Fourier shell correlation (FSC) with a generous, soft-edged solvent mask and high-resolution noise substitution in RELION PostProcess, resulting in a resolution estimate of 3.9 Å with a 0.5 FSC cutoff.

### Atomic Model Building

The highest resolution structure of an AD-brain derived tau PHF (PDB 6HRE) was used as a starting model for atomic model building. Coordinates were docked in the 3.9 Å electron density map as a rigid body, and minor fitting was performed in COOT⁴³. EGCG was modeled into the electron density by first rigid body docking from molecule KDH 911 of PDB 4AWM and subsequent real space refinement. Fibril layers were modeled to maintain local contacts between chains in the fibril during structure refinement. We performed automated structure refinement using phenix.real_space_refine⁴⁴.

### Inhibitor Screening in Tau Biosensor Cells

HEK293 cell lines stably expressing tau-K18 P301 S-eYFP were engineered by Marc Diamond's lab at UTSW¹⁷ and used without further characterization or authentication. Cells were maintained in DMEM (Life Technologies, cat. 11965092) supplemented with 10% (vol/vol) FBS (Life Technologies, cat. A3160401), 1% penicillin/streptomycin (Life Technologies, cat. 15140122), and 1% Glutamax (Life Technologies, cat. 35050061) at 37° C., 5% CO2 in a humidified incubator. Fibrils and patient-derived seeds were incubated for 16 hours with indicated inhibitor to yield a final inhibitor concentration of 10 μM (on the biosensor cells), except for IC50 determinations, which instead used adjustments to achieve the final indicated inhibitor concentration. Note that the IUPAC name of the chalcone inhibitor used is 3-(4-methylphenyl)-1-(3-nitrophenyl)-prop-2-en-1-one. For seeding, inhibitor-treated seeds were sonicated in a cuphorn water bath for 3 minutes, and then mixed with 1 volume of Lipofectamine 3000 (Life Technologies, cat. 11668027) prepared by diluting 1 μl of Lipofectamine in 19 μl of OptiMEM. After twenty minutes, 10 μl of fibrils were added to 90 μl of tau biosensor cells. The number of seeded aggregates was determined by imaging the entire well of a 96-well plate in triplicate using a Celigo Image Cytometer (Nexcelom) in the YFP channel. Aggregates were counted using ImageJ⁴⁵ by subtracting the background fluorescence from unseeded cells and then counting the number of peaks with fluorescence above background using the built-in Particle Analyzer. The number of aggregates was normalized to the confluence of each well, and dose-response plots were generated by calculating the average and standard deviations from triplicate measurements. For high quality images, cells were photographed on a ZEISS Axio Observer D1 fluorescence microscope using the YFP fluorescence channel.

### ThT Aggregation Kinetics Assay

Concentrated tau-K18+ was diluted into PBS buffer (pH 7.4) to a final concentration of 20 μM with an equimolar ratio of indicated small molecule inhibitor. Proteins were shaken in solutions containing 40 μM ThT, 0.225 mg/ml heparin (Sigma cat. H3393), and 1 mM DTT in a 96-well plate with a plastic bead to enhance agitation. ThT fluorescence was measured with excitation and emission wavelengths of 440 and 480 nm, and averaged curves were generated from triplicate measurements as indicated in the figure legend. Error bars show the standard deviation of replicates measurements. For endpoint ThT

### Peptide Fibril Formation and ThT Measurements

PHFE2 peptide was disaggregated in water to a concentration of 120 mg/ml (80 mM), and the concentrated peptide stock was subsequently diluted to 100 mg/ml (66 mM) by addition of water and 10×PBS (to a final concentration of 1×PBS). Fibrils were grown at 37° C. in an orbital shaker with shaking at 350 rpm for 3-4 weeks. Solutions were removed from shaking and heat upon appearance of fibrils, confirmed by negative stain EM. For ThT experiments, fibrils were diluted to 30 mM in PBS, and ThT was added to a final concentration of 40 μM. EGCG was added from a concentrated stock to a final concentration of 5 mM where indicated. ThT measurements were carried out as described above, except readings were taken from a 384-well plate with a volume of 35 μl.

### REFERENCES FOR EXAMPLES 1 AND 2

1. Boyer, D. R. et al. Structures of fibrils formed by α-synuclein hereditary disease mutant H50Q reveal new polymorphs. Nature Structural & Molecular Biology 26, 1044-1052, doi:10.1038/s41594-019-0322-y (2019).
2. Gallagher-Jones, M. et al. Sub-ångström cryo-EM structure of a prion protofibril reveals a polar clasp. Nature structural & molecular biology 25, 131-134, doi:10.1038/s41594-017-0018-0 (2018).
3. Guerrero-Ferreira, R. et al. Two new polymorphic structures of alpha-synuclein solved by cryo-electron microscopy. bioRxiv, 654582, doi:10.1101/654582 (2019).
4. Iadanza, M. G. et al. The structure of a β2-microglobulin fibril suggests a molecular basis for its amyloid polymorphism. Nature Communications 9, 4517, doi:10.1038/s41467-018-06761-6 (2018).
5. Li, B. et al. Cryo-EM of full-length α-synuclein reveals fibril polymorphs with a common structural kernel. Nature communications 9, 3609-3609, doi:10.1038/s41467-018-05971-2 (2018).
6. Falcon, B. et al. Structures of filaments from Pick's disease reveal a novel tau protein fold. Nature 561, 137-140, doi:10.1038/s41586-018-0454-y (2018).
7. Falcon, B. et al. Tau filaments from multiple cases of sporadic and inherited Alzheimer's disease adopt a common fold. Acta neuropathologica 136, 699-708, doi:10.1007/s00401-018-1914-z (2018).
8. Falcon, B. et al. Novel tau filament fold in chronic traumatic encephalopathy encloses hydrophobic molecules. Nature 568, 420-423, doi:10.1038/s41586-019-1026-5 (2019).
9. Fitzpatrick, A. W. P. et al. Cryo-EM structures of tau filaments from Alzheimer's disease. Nature 547, 185-190, doi:10.1038/nature23002 (2017).
10. Kollmer, M. et al. Cryo-EM structure and polymorphism of A amyloid fibrils purified from Alzheimer's brain tissue. Nature Communications 10, 4760, doi:10.1038/s41467-019-12683-8 (2019).
11. Andrich, K. & Bieschke, J. The Effect of (−)-Epigallo-catechin-(3)-gallate on Amyloidogenic Proteins Suggests a Common Mechanism. Adv Exp Med Biol 863, 139-161, doi:10.1007/978-3-319-18365-7_7 (2015).
12. Bieschke, J. et al. EGCG remodels mature α-synuclein and amyloid-O fibrils and reduces cellular toxicity. Proceedings of the National Academy of Sciences 107, 7710-7715, doi:10.1073/pnas.0910723107 (2010).
13. Chirita, C., Necula, M. & Kuret, J. Ligand-Dependent Inhibition and Reversal of Tau Filament Formation. Biochemistry 43, 2879-2887, doi:10.1021/bi036094h (2004).
14. Kocisko, D. A. et al. New Inhibitors of Scrapie-Associated Prion Protein Formation in a Library of 2,000 Drugs and Natural Products. Journal of Virology 77, 10288, doi:10.1128/JVI.77.19.10288-10294.2003 (2003).
15. Townsend. D. et al. Epigallocatechin-3-gallate remodels apolipoprotein A-I amyloid fibrils into soluble oligomers in the presence of heparin. The Journal of biological chemistry 293, 12877-12893, doi:10.1074/jbc.RA 118.002038 (2018).
16. Clavaguera, F. et al. Brain homogenates from human tauopathies induce tau inclusions in mouse brain. Proceedings of the National Academy of Sciences of the United States of America 110, 9535-9540, doi:10.1073/pnas.1301175110 (2013).
17. Sanders, David W. et al. Distinct Tau Prion Strains Propagate in Cells and Mice and Define Different Tauopathies. Neuron 82, 1271-1288, doi:http://dx.doi.org/10.1016/j.neuron.2014.04.047 (2014).
18. Eisenberg, D. & Jucker, M. The amyloid state of proteins in human diseases. Cell 148, 1188-1203, doi:10.1016/j.cell.2012.02.022 (2012).
19. Wiltzius, J. J. W. et al. Molecular mechanisms for protein-encoded inheritance. Nat Struct Mol Biol 16, 973-978, doi:http://www.nature.com/nsmb/journal/v16/n9/suppinfo/nsmb.1643_S1.html (2009).
20. Tsemekhman, K., Goldschmidt, L., Eisenberg, D. & Baker, D. Cooperative hydrogen bonding in amyloid formation. Protein science: a publication of the Protein Society 16, 761-764, doi:10.1110/ps.062609607 (2007).
21. Michalska, K. et al. Structure of Calcarisporiella thermophila Hsp104 Disaggregase that Antagonizes Diverse Proteotoxic Misfolding Events. Structure 27, 449-463.c447, doi:https://doi.org/10.1016/j.str.2018.11.001 (2019).
22. Shorter, J. & Lindquist, S. Hsp104, Hsp70 and Hsp40 interplay regulates formation, growth and elimination of Sup35 prions. The EMBO Journal 27, 2712-2724, doi:10.1038/emboj.2008.194 (2008).
23. Jackrel, M. E. & Shorter, J. Reversing deleterious protein aggregation with re-engineered protein disaggregases. Cell Cycle 13, 1379-1383, doi:10.4161/cc.28709 (2014).
24. Tariq, A. et al. Mining Disaggregase Sequence Space to Safely Counter TDP-43, FUS, and & #x3b1; —Synuclein Proteotoxicity. Cell Reports 28, 2080-2095.c2086, doi:10.1016/j.celrep.2019.07.069 (2019).
25. Eisele, Y. S. et al Targeting protein aggregation for the treatment of degenerative diseases. Nature reviews. Drug discovery 14, 759-780, doi:10.1038/nrd4593 (2015).
26. Kowalinski, E. et al. Structural analysis of specific metal chelating inhibitor binding to the endonuclease domain of influenza pH IN 1 (2009) polymerase. PLoS Pathog 8, e100283l-e1002831, doi:10.1371/joumal.ppat.1002831 (2012).
27. Miyata, M. et al. The Crystal Structure of the Green Tea Polyphenol (−)-Epigallocatechin Gallate-Transthyretin Complex Reveals a Novel Binding Site Distinct from the Thyroxine Binding Site. Biochemistry 49, 6104-6114, doi:10.1021/bi1004409 (2010).
28. Robertson, 1. M., Li, M. X. & Sykes, B. D. Solution structure of human cardiac troponin C in complex with the green tea polyphenol, (−)-epigallocatechin 3-gallate. The Journal of biological chemistry 284, 23012-23023, doi:10.1074/jbc.M109.021352 (2009).
29. Skrzypczak-Jankun, E., Zhou, K. & Jankun, J. Inhibition of lipoxygenase by (−)-epigallocatechin gallate: X-ray analysis at 2.1 Å reveals degradation of EGCG and shows soybean LOX-3 complex with EGC instead. Int J Mol Med 12, 415-420 (2003).
30. Urusova, D. V. et al. Epigallocatechin-gallate suppresses tumorigenesis by directly targeting Pin1. Cancer Prev Res (Phila) 4, 1366-1377, doi:10.1158/1940-6207.CAPR-11-0301 (2011).
31. Baell, J. B. Feeling Nature's PAINS: Natural Products, Natural Product Drugs, and Pan Assay Interference Compounds (PAINS). Journal of Natural Products 79, 616-628, doi:10.1021/acs.jnatprod.5b00947 (2016).
32. Negri, A., Naponelli, V., Rizzi, F. & Bettuzzi, S. Molecular Targets of Epigallocatechin-Gallate (EGCG): A Special Focus on Signal Transduction and Cancer. Nutrients 10, 1936, doi:10.3390/nu10121936 (2018).
33. Seidler, P. M. et al. Structure-based inhibitors halt prion-like seeding by Alzheimer's disease- and tauopathy-derived brain tissue samples. Journal of Biological Chemistry, doi:10.1074/jbc.RA 119.009688 (2019).
34. Arakhamia, T. et al. Posttranslational Modifications Mediate the Structural Diversity of Tauopathy Strains. Cell, doi:10.1016/j.cell.2020.01.027.
35. Boyer, D. R. & Eisenberg, D. S. A structure-based model for the electrostatic interaction of the N-terminus of protein tau with the fibril core of Alzheimer's Disease filaments. bioRxiv, 484279, doi:10.1101/484279 (2018).
36. Röder, C. et al. Atomic structure of PI3-kinase SH3 amyloid fibrils by cryo-electron microscopy. Nature Communications 10, 3754, doi:10.1038/s41467-019-11320-8 (2019).
37. Kaufman, S. K., Del Tredici, K., Thomas, T. L., Braak, H. & Diamond, M. I. Tau seeding activity begins in the transentorhinal/entorhinal regions and anticipates phospho-tau pathology in Alzheimer's disease and PART. Acta Neuropathologica 136, 57-67, doi:10.1007/s00401-018-1855-6 (2018).
38. Carragher, B. et al. Leginon: An Automated System for Acquisition of Images from Vitreous Ice Specimens. J Struct Biol 132, 33-45, doi:https://doi.org/10.1006/jsbi.2000.4314 (2000).
39. Rohou, A. & Grigorieff, N. CTFFIND4: Fast and accurate defocus estimation from electron micrographs. J Struct Biol 192, 216-221, doi:10.1016/j.jsb.2015.08.008 (2015).
40. Grant, T. & Grigorieff, N. Measuring the optimal exposure for single particle cryo-EM using a 2.6 Å reconstruction of rotavirus VP6. eLife 4, e06980-e06980, doi:10.7554/eLife.06980 (2015).
41. Tang, G. et al. EMAN2: An extensible image processing suite for electron microscopy. J Struct Biol 157, 38-46, doi:https://doi.org/10.1016/j.jsb.2006.05.009 (2007).
42. Scheres, S. H. W. RELION: Implementation of a Bayesian approach to cryo-EM structure determination. J Struct Biol 180, 519-530, doi:https://doi.org/10.1016/j.jsb.2012.09.006 (2012).
43. Emsley, P., Lohkamp, B., Scott, W. G. & Cowtan, K. Features and development of Coot. Acta Crystallographica Section D 66, 486-501, doi:doi:10.1107/S0907444910007493 (2010).
44. Afonine, P. V. et al. Real-space refinement in PHENIX for cryo-EM and crystallography. Acta Crystallographica Section D 74, 531-544, doi:doi:10.1107/S2059798318006551 (2018).
45. Schneider. C. A. R., W. S. & Eliceiri, K. W. NIH Image to ImageJ: 25 years of image analysis. Nature methods 9, 671-675 (2012).

### Example 3: Tables for Examples 1 and 2

TABLE 1
Description of brain tissue donor characteristics.

| Brain | | | Age at | Neuropath | |
|---|---|---|---|---|---|
| Bank | | Sex | death | Diagnosis | Brain Region |
| MAYO | AD1 | M | 68 | AD | Middle frontal |
| UCLA | AD2 | F | 78 | AD | Frontal cortex |
| | AD3 | M | 71 | AD | Frontal cortex |
| | AD4 | F | 62 | AD | Hippocampus |
| | AD5 | F | 91 | AD | Hippocampus |
| | AD6 | M | 75 | AD | Cerebellum |
| UCSF | AD7 | F | 58 | AD | Inferior temporal gyrus |
| | AD8 | F | 69 | AD | Inferior temporal gyrus |
| | AD9 | M | 70 | AD | Inferior temporal gyrus |
| | AD10 | F | 65 | AD | Inferior temporal gyrus |
| | AD11 | M | 72 | AD | Inferior temporal gyrus |
| | AD12 | M | 77 | AD | Inferior temporal gyrus |
| | AD13 | M | 66 | AD | Inferior temporal gyrus |
| | AD14 | F | 64 | AD | Inferior temporal gyrus |
| | AD15 | M | 68 | AD | Inferior temporal gyrus |
| UCSF | PSP1 | F | 76 | PSP | Inferior frontal gyrus |
| | PSP2 | F | 78 | PSP | Inferior frontal gyrus |
| | PSP3 | F | 72 | PSP | Inferior frontal gyrus |
| | PSP4 | F | 69 | PSP | Inferior frontal gyrus |
| | PSP5 | M | 71 | PSP | Inferior frontal gyrus |
| | PSP6 | F | 70 | PSP | Inferior frontal gyrus |
| | PSP7 | F | 71 | PSP | Inferior frontal gyrus |

In Table 1, for donors with multiple brain regions assayed, subsequent cells with sex, age and diagnosis are left blank and are the same as those given in the nearest row above.

TABLE 2
Cryo-EM data collection, refinement, and validation statistics.

| | Tau Paired helical filament |
|---|---|
| | from Alzheimer's disease |
| Name | brain complexed with EGCG |
| PDB ID | PDB ID 6W9B |
| EMDB ID | EMD-21581 |
| Data collection | |
| Magnification | ×130,000 |
| Defocus range (um) | 1.8-2.25 |
| Voltage (kV) | 300 |
| Camera | K2 Summit |
| | (Quantum LS) |
| Frame exposure time (s) | 0.2 |
| # movie frames | 30 |
| Total electron dose (e−/Å²) | 37.8 |
| Pixel size (Å) | 1.07 |
| Reconstruction | |
| Box size (pixel) | 432 |
| Inter-box distance (Å) | 43.2 |
| # micrograph collected | 6,988 |
| # segments extracted | 137,008 |
| # segments after Class2D | N/A |
| # segments after Class3D | 19,995 |
| Resolution (Å) | 3.9 |
| Map sharpening B-factor (Å²) | 0 |
| Helical rise (Å) | 2.41 |
| Helical twist (°) | 179.49 |
| Point Group | C₁ |
| Atomic model | |
| # non-hydrogen atoms | 2480 |
| # protein residues | 304 |
| R.m.s.d. bonds (Å) | |
| R.m.s.d. angles (°) | |
| Molprobity clashscore, all atoms | 11.1 |
| Molprobity score | 2.1 |
| Poor rotamers (%) | 0 |
| Ramachandran outliers (%) | 0 |
| Ramachandran allowed (%) | 9.0 |
| Ramachandran favored (%) | 91.0 |
| Cβ deviations > 0.25 Å (%) | 0 |
| Bad bonds (%) | 0 |
| Bad angles (%) | 0 |

### Example 4: Improved Lead Compounds

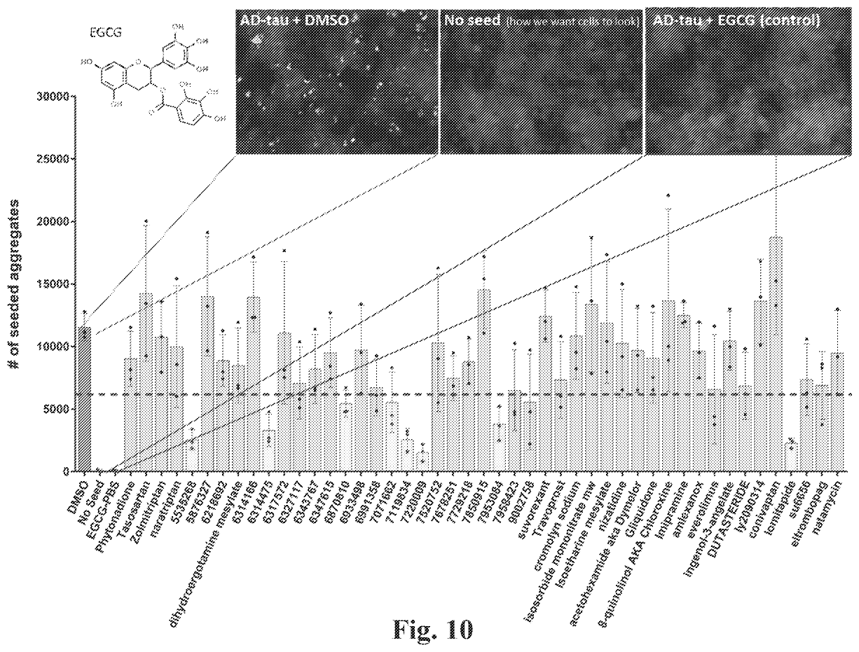

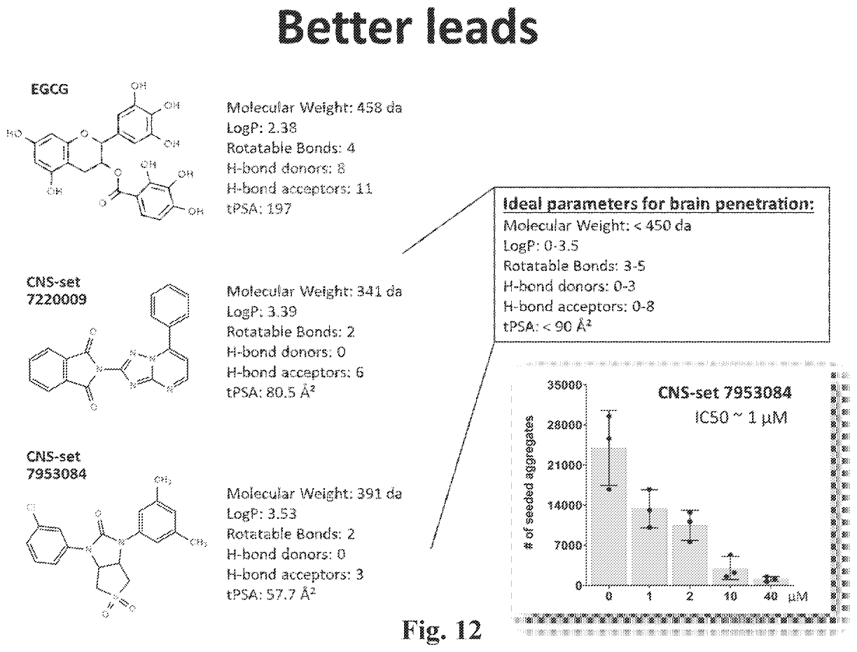

Additional supporting evidence that the pharmacophore that is defined by EGCG can be used to discover tau inhibitor molecules that have improved drug-like properties is provided in FIG. 10, FIG. 11, and FIG. 12. In Example 1, we propose that in silico docking can lend to the discover of new alternative inhibitors with more promising chemical scaffolds. In FIG. 10. FIG. 11, and FIG. 12, we show data to support this. We experimentally tested 50 compounds that we identified by in silico docking to the pharmacophore that is defined by EGCG and confirmed that 11 of these compounds inhibit seeding by AD-tau. All the new leads have superior lead-like properties compared to EGCG. As seen in FIG. 12, based on the properties of the new lead molecules, these compounds are expected to enter the brain whereas, without modification, EGCG is not. None of the lead molecules inhibit seeding quite as potently as EGCG. EGCG has an IC50 of around 100-300 nM, and our new leads have IC50s in the 1-10 uM range. But the leads are a unique starting point given their intrinsic drug-like properties. Beginning with these molecules is advantageous, which have some capacity to enter the brain. We expect we are 1 or 2 H-bonds away from a molecule that inhibits as potently as EGCG, and that an analog with H-bond donors and acceptors in the right place will do the trick.

### Example 5: Catalytic Synthesis of PEGylated EGCG Conjugates that Disaggregate Alzheimer's Tau

The naturally occurring flavonoid (−)-epigallocatechin gallate (EGCG) is a potent disaggregant of tau fibrils. Guided by the recent cryo-electron microscopy (cryoEM) structure of EGCG bound to fibrils of tau derived from an Alzheimer's brain donor, we report methods to site-specifically modify the EGCG D-ring with aminoPEGylated linkers. The resultant molecules inhibit tau fibril seeding in Alzheimer's brain extracts. Formulations of aminoPEGylated EGCG conjugated to the (quasi)-brain-penetrant nanoparticle Ferumoxytol inhibit seeding by AD-tau with linker length affecting activity. The protecting group free catalytic cycloaddition of amino azides to mono propargylated EGCG described here provides a blueprint for access to stable nanoparticulate forms of EGCG potentially useful as therapeutics to eliminate Alzheimer's-related tau tangles.

Alzheimer's disease (AD) is the 6^{th} leading cause of death in the United States and 7^{th} in the world. Personal and economic burdens associated with this most common type of dementia are enormous. Approximately 6.8 million Americans currently suffer from the disease. By 2050, its annual costs to the healthcare system are anticipated to reach $1.1 trillion.² Despite decades of research and numerous attempts at treatment, much is still unknown about the etiology of Alzheimer's. Two main markers have been identified: plaques of aggregated β-amyloid and neurofibrillary tangles of tau. However, the precise cause of cognitive decline and effective drug targets have been elusive. Early focus on β-amyloid led to clinical trials of multiple therapeutic candidates with limited success.³ Those failed trials called into question the hypothesis that amyloid plaques play a decisive role in cognitive decline. Recent advances in imaging⁴ showed tau tangles to be the best predictors of Alzheimer's progression as well as the species responsible for driving brain atrophy. Oligomeric and fibrillar tau appear to be promising targets for therapeutics.

The polyphenolic flavanoid (−)-epigallocatechin gallate (EGCG) inhibits aggregation of proteins involved in neurogenerative amyloidoses including huntingtin, amyloid-β and α-synuclein.⁵ Wobst et al⁶ reported EGCG blocks the fibrillization of tan by sequestering unfolded protein monomers. Recently, cryoEM was used to determine the binding site for EGCG on fibrils of tau deriving from the brain tissue of a donor with AD.⁷ Relative to the apo AD-tau fibril, the bound form contains EGCG wedged into an interfacial cleft (FIG. 14).

The structure of EGCG bound to AD tau indicated positions on the small molecule that might serve as anchor points for nanoparticle conjugation, wherein the ability to bind tau fibrils would be retained. Despite numerous reports of therapeutic potential for EGCG, the compound is prone to auto-oxidation, has poor pharmacokinetics and is largely excluded from the brain when administered systemically. Stable conjugation to brain penetrant nanoparticles was seen as potential means to offset those limitations.⁸ We selected Ferumoxytol as a nanoparticle carrier. Ferumoxytol exhibits moderate brain penetration, with penetration increasing coincident with pathologies that alter the neurovascular unit.

As best modeled (FIG. 14B), the tau bound form of EGCG oriented its A-ring C5 phenol and a major portion of the gallate D-ring towards solvent. We sought to selectively derivatize the natural product ($17/g, Oakwood Chemicals) at one position along this periphery (see Scheme 1) with end-functionalized ethylene glycol chains of varying length.

Wang and co-workers had reported that a sodium salt of EGCG reacted with propargyl bromide in DMF at 80° C. to afford predominately A-ring mono-ether 2, along with lesser amounts of further propargylated compounds.⁹ We repeated this reaction and found spectroscopic data for the major etherification product was inconsistent with structure 2. HMBC spectra showed a correlation between the propargylic methylene protons (OCH₂, 4.76 ppm) and C-4″ (137.0 ppm). C4″ exhibited coupling with C2″-H (6.89 ppm), and C2″-H also correlated to the carbonyl carbon (165.7 ppm). C5 (95.2 ppm), the linkage site assigned in 2, exhibited correlations to C6-H and the C4 methylene protons, but not to the propargyl group or D ring aryl protons. These data indicated the proper structure assignment should be ether 3, wherein alkylation had occurred at the para phenol of the gallate ester. This phenol is presumably the most acidic in EGCG.

A second product isolated from the reaction was doubly etherified (see SI for structure) and showed HMBC correlations between a second propargylic methylene (OCH₂, 4.67 ppm) and C4′ (137.0 ppm), and between C4′ and C2′-H (6.53 ppm). Data indicated the second propargyl ether formed on Ring C. The phenols on Ring A appeared to be the least susceptible to alkylation under basic conditions.

With the structure of 3 confirmed, conditions were screened to optimize its formation while avoiding the use of NaH in DMF—a potentially explosive combination, particularly when heated.¹⁰ It was eventually found that treating EGCG with 1 eq. propargyl bromide and 0.5 eq. powdered K₂CO₃ in DMF at room temperature afforded 3 in 45% isolated yield—versus the 33% yield obtained using the NaH/DMF procedure.

We next synthesized a set of glycol based ω-amino azides with chain lengths varying from 5 to 17 atoms (4a-e, see Table 4 and experimental section for details) in order to produce EGCG conjugates with incrementally increasing chain lengths. Cycloaddition reaction conditions were first optimized with 4c using copper catalysis (Table 3).¹¹ Standard conditions¹² using catalytic Cu(II) and sodium ascorbate in aqueous THF (Table 3, entries 1 & 2), failed to cycloadd 4c to 3 due to substrate insolubility. When THF was replaced with tBuOH, desired triazole 5c was detected, but only in trace quantities (entry 3). An attempt to replace sodium ascorbate with Cu°¹³ was unsuccessful (entry 4), as was the use of stoichiometric Cu(I) (entry 6). Notably, when stoichiometric amounts of CuSO₄ were employed (entry 5), starting materials were consumed and a highly insoluble precipitate formed. The use of H₂O/DMSO cosolvent mixture resulted in the formation of the product in 12% yield (entry 7), which remained unchanged even in the presence of excess aminoazide partner (entry 8). We suspected this material was a copper/product complex and hypothesized that earlier attempts at catalysis may have been poisoned by amino polyphenol 5c. To fortify the copper catalyst against possible product sequestration, we turned to polydentate ligands reported by Sharpless.¹⁴ Gratifyingly, in the presence tris((I-benzyl-4-triazolyl)methyl)amine (TBTA), 20 mol % of CuSO₄ rapidly catalyzed the cycloaddition of 4c to 3 in H₂O/DMSO at room temperature to afford triazole adduct 5c in 52% isolated yield (entry 9). When the catalyst load was decreased from 20 to 5 mol %, isolated yield decreased, and conversion plateaued at roughly 80%. The reactions required no workup and product was easily purified as its TFA salt via preparative reverse-phase HPLC (see experimental section for details).

Using the conditions shown in Table 3, entry 9, a set of EGCG conjugates having increasing chain lengths were synthesized (Table 4). As the number of glycol units increased, the yield of triazole products 5 decreased slightly. But in all cases, analytically pure product was isolated readily using preparative reversed phase HPLC.

Interestingly, when analyzing amine salts 5 by ¹H NMR in protic solvents (i.e. CD₃OD or D₂O), the aryl protons in the A-ring (5.93 ppm) quickly disappeared. Their integration (relative to stable resonances) decreased ˜75% in 3 hours. Overnight storage of the NMR samples saw complete disappearance of both signals. HRMS identified the products as [M+1]+2 ions, indicating C—H bonds in the A ring had been replaced by C-D bonds. Notably. C—H bonds in the C and D rings showed no exchange, even after prolonged storage. Deuteration of flavonoids has been observed in the gas phase by mass spectrometry.¹⁵ Jordheim and coworkers reported anthocyanidin natural products are deuterated in 15 vol % TFA in CD₃OD over a period of days.¹⁶ Rapid deuteration of compounds 5 at room temperature may derive from the acidity of their amine salt appendages, wherein deuteration of the A-ring was presumably occurring via a dearomatized species of type i (Scheme 2). The A-ring appeared to be considerably more basic than the C and D rings. Along those lines, we observed that EGCG itself would react with N-iodo succinimide to rapidly and selectively iodinate the A-ring (data not shown).

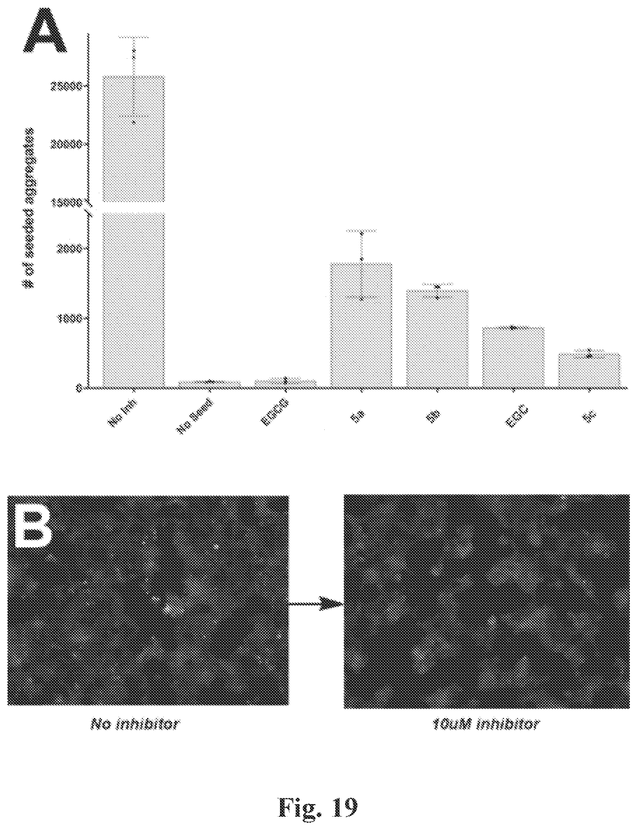

We next tested if the D-ring site of triazole-linked amino PEGylation would interfere with tau fibril disaggregation observed for EGCG. AD crude brain extracts have been shown to seed aggregation of fluorescently labeled tau in HEK293 recipient biosensor cells expressing an aggregation-prone fragment of tau called K18¹⁷, and seeding is inhibited by EGCG.^{7a} We compared inhibition of seeding by EGCG and D-ring analogs (5a-c) as a preliminary proof-of-concept. Crude extract of autopsied brain tissue of a donor with AD (prepared as described in the SI) were pre-incubated with inhibitors (10 μM final concentration on cells) for 16-18 hours and resulting homogenates were added to the cells for imaging 3 days later. The data obtained is shown in FIG. 19.

Intracellular tau aggregates are seen as bright green puncta in cells that were seeded with crude AD brain extract in the absence of inhibitor. The number of puncta in inhibitor-treated cells are a proxy used to assess the disaggregating activity of EGCG-linked nanoparticles. To our delight, all of the EGCG-linker conjugates inhibited seeding by AD brain extracts by at least 90% with 5c displaying potency nearly on par with EGCG itself. As a comparison with other analogs of EGCG, we tested ECG, which lacks the meta-OH group of the C ring. Consistent with the structure of EGCG bound to tau, which shows no contact with the meta-OH, ECG was seen to inhibit seeding as well as the linker-conjugated analogs and nearly as well as the parent natural product, EGCG. These data demonstrate that D ring derivatizations are well tolerated, consistent with our observation that the D ring remains largely solvent-exposed in the binding cleft of tangled tau filaments from AD brain.

EGCG is subject to off-target binding and rapid metabolism, which restricts its therapeutic potential. We reason that covalent conjugation of EGCG to nanoparticles may reduce binding to metabolic and off-target proteins, which accommodate EGCG inside of buried active sites of globular proteins that are sterically inaccessible to nanoparticle-bound molecules of EGCG. Thus, we sought to synthesize a series of EGCG-nanoparticle conjugates that varied by linker length to identify a minimal linker that retains interaction of EGCG with the solvent exposed binding cleft of fibrillar tau.

Ferumoxytol is an FDA approved carbohydrate-coated iron nanoparticle with widespread use in the clinic with applications ranging from anemia treatment to off-label MR imaging of neurovasculature.¹⁸ We conjugated an expanded series of EGCG bearing linkers of incrementally increasing length, 5a-e, to Ferumoxytol nanoparticles using standard amidation conditions (sulfo-NHS, EDC, 2 h, rt). Unlike previous inclusion-based, labile EGCG nanoparticle formulations that release EGCG at sites of action,¹⁹ we loaded the small molecule via covalent attachment. Covalent conjugation is likely to reduce off-target binding and has added potential to improve potency by exploiting the multivalency of the nanoparticle (each nanoparticle displays ˜50 potential linking sites).

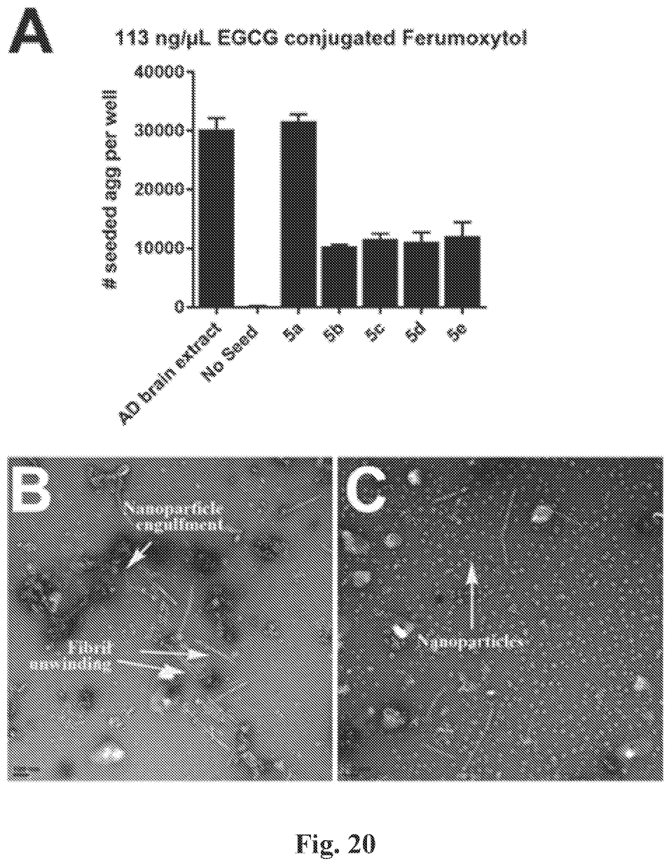

We tested the activity of Ferumoxytol conjugated EGCG analogs using the biosensor cell assay described above, except we omitted the pre-incubation step such that our assay more closely resembled the scenario of therapeutic intervention, for which there is no pre-incubation period. Nanoparticle conjugated EGCG derivative was mixed with crude AD brain extract and immediately transfected into tau biosensor cells. We find that all the analogs except for the compound with the shortest linker (5a) exhibited desired activity inhibiting seeding by at least 50% (FIG. 20A). Overall, our data demonstrates that nanoparticle conjugates retain the inhibitory properties of the parent compound, and underscores that functional EGCG nanoparticles can be successfully designed based on information that is gleaned from the cryoEM structure.

As added evidence of its inhibitory action, we also observed an interesting effect of EGCG nanoparticle incubation with tau paired helical filaments purified from AD brain by negative-stain electron microscopy. Nanoparticles loaded with 5c form dense clouds that engulf AD-tau fibrils, in some cases apparently unwinding the paired helical filament (FIG. 20B). Non-conjugated control nanoparticles exhibited no apparent interaction with AD-tau fibrils (FIG. 20C).

In summary, we have optimized a monoetherification of naturally occurring EGCG and properly assigned the regiochemistry of the reaction. We have established a procedure to directly catalyze cycloaddition of glycol based w-amino azide chains to this molecule. The resultant amino polyphenolic conjugates retain the ability to disaggregate AD brain-derived tau-both as isolated species and when loaded onto Ferumoxytol nanoparticles. These promising results provide a blueprint for future work wherein further refinements to the EGCG molecule and optimized nanoparticulate formulations could provide means to deliver a potent tau fibril disaggregant to the brains of Alzheimer's patients.

### Tables

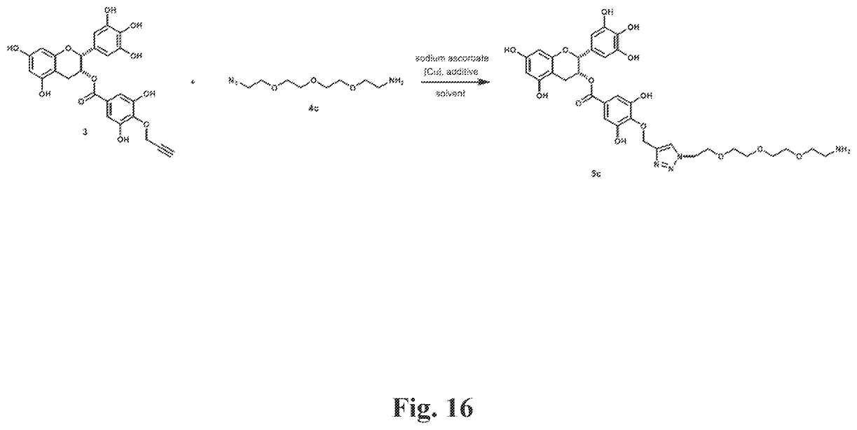

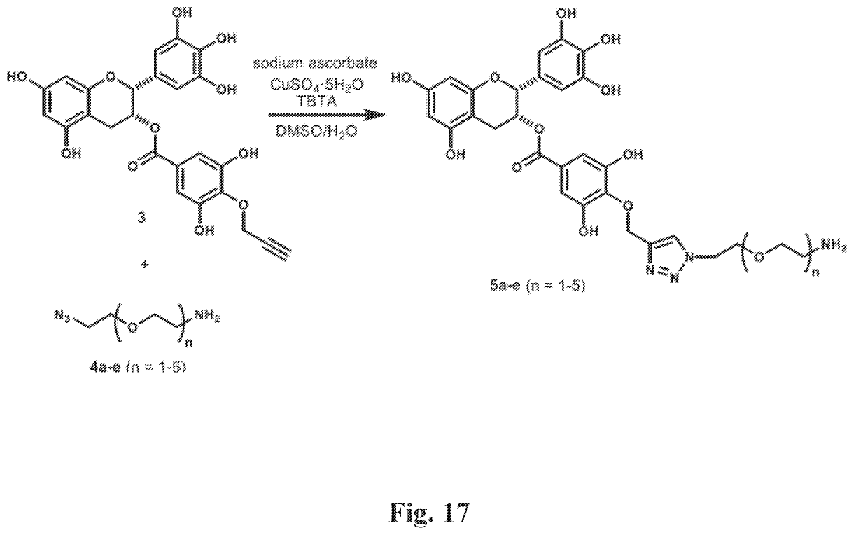

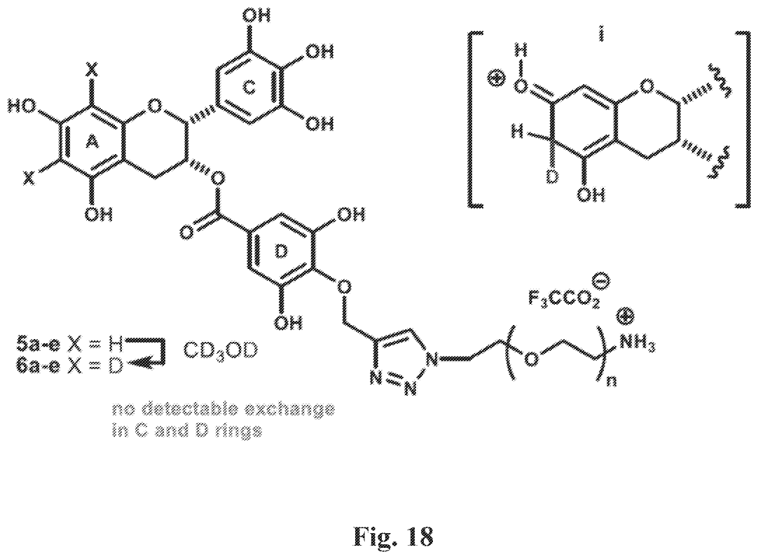

TABLE 3
Protecting group free, copper catalyzed
Huisgen cycloaddition to form aminoPEGylated EGCG^{a}
(Reaction shown in FIG. 16)

| Entry | [Cu] | Additive | Solvent (4:1) | Yield^{b} |
|---|---|---|---|---|
| 1 | Cu(OAc)₂ (0.2 eq.) | — | H₂O/THF | 0% |
| 2 | CuSO₄ · 5H₂O (0.2 eq.) | — | H₂O/THF | 0% |
| 3 | CuSO₄ · 5H₂O (0.2 eq.) | — | H₂O/^{t}BuOH | trace |
| 4^{c} | CuSO₄ · 5H₂O (0.2 eq.) | — | H₂O/^{t}BuOH | 0% |
| 5 | CuSO₄ · 5H₂O (1 eq.) | — | H₂O/^{t}BuOH | —^{d} |
| 6^{e} | CuBr (1 eq.) | — | H₂O/DMSO | —^{f} |
| 7 | CuSO₄ · 5H₂O (0.5 eq.) | — | H₂O/DMSO | 12% |
| 8^{g} | CuSO₄ · 5H₂O (0.5 eq.) | — | H₂O/DMSO | 12% |
| 9 | CuSO₄ · 5H₂O (0.2 eq.) | TBTA^{h} | H₂O/DMSO | 52% |

^{a}Reaction conditions: 3 (1 mmol), 4c (1 mmol), [Cu], additive (50 mol %), sodium ascorbate (2.0 eq.), solvent (0.1 M), 1 h.
^{b}Isolated yield
^{c}Cu (powder) was used as a reductant instead of sodium ascorbate
^{d}Reaction resulted in the formation of insoluble precipitate
^{e}No sodium ascorbate
^{f}Complex mixture
^{g}2 mmol of 4c was used
^{h}TBTA = tris((1-benzyl-4-triazolyl)methyl)amine

TABLE 4
A set of EGCG derivatives
having varied linker lengths^{a}
(Reaction shown in FIG. 17)

| Compound | n | Yield (%)^{b} |
|---|---|---|
| 5a | 1 | 68 |
| 5b | 2 | 58 |
| 5c | 3 | 52 |
| 5d | 4 | 46 |
| 5e | 5 | 45 |

^{a}Reaction conditions: 3 (1 mmol), 4 (1 mmol), CuSO₄ · 5H₂O (0.2 eq.), TBTA (50 mol %), sodium ascorbate (2.0 eq.), DMSO/H₂O (4:1) (0.1 M), 1 h.
^{b}Isolated yield

### Materials and Experiments

All reagents were purchased from commercial suppliers (Sigma-Aldrich. Combi-Blocks or Oakwood Chemicals) and were used without further purification. When necessary, reaction solvents were dried using an activated alumina solvent drying system. Thin layer chromatography (TLC) was performed on pre-coated plates Sorbent Technologies, silica gel 60 PF₂₃₄ (0.25 mm). TLC were visualized with UV light (254 nm) or stained using KMnO₄ or ninhydrin. Flash chromatography was performed on silica gel 60 (240-400 mesh). Purification of final products was performed using an Agilent 1200 HPLC system equipped with G1361A preparative pumps, and a Waters Sunfire C18 column (5 μm, 19 mm×250 mm). Analytical HPLC was performed using the same system, but with a G1312A binary pump. NMR spectra were recorded on a Bruker Avance (500 MHz) spectrometer using CDCl₃ or CD₃OD as solvents and referenced relative to residual CHCl₃ (δ=7.26 ppm) or CD₃OD (δ=3.31 ppm). Chemical shifts are reported in ppm and coupling constants (J) in Hertz. 13C NMR was recorded on the same instruments (125 MHz) with total proton decoupling referenced relative to residual CHCl₃ (8=77.16 ppm) or CD₃OD (δ=49.00 ppm). Infrared spectra were obtained on Jasco FT/IR-4100, both are equipped with a universal ATR sampling accessory. High-resolution mass spectra were recorded on Waters LCT Premier. Optical rotations were measured on a Rudolph Autopol III Automatic Polarimeter and are quoted in units of 10⁻¹ deg cm² g⁻.

### Preparation of Amino Azides 4; General Procedure (GP1).

The corresponding diol (1.0 eq.) was dissolved in DCM (0.6 M), followed by the addition of TsCl (2.1 eq.). The reaction was cooled to 0° C. KOH (8.0 eq.) was then added in one portion, the reaction was warmed to room temperature and stirred for 4 hours. The mixture was then diluted with H₂O (100 mL) and extracted with DCM (3×100 mL). The combined organic layers were dried (MgSO₄), filtered and concentrated in vacuo. The crude white solid was used directly in the next step.

To the crude bis-tosylate (1.0 eq.) in DMF (0.6 M) under Ar was added NaN₃ (4.0 eq.). The reaction was stirred overnight at 80° C. The mixture was then cooled to room temperature, diluted with H₂O (100 mL) and extracted with EtOAc (3×100 mL). Combined organic layers were washed with H₂O (2×50 mL), brine (2×50 mL), dried (MgSO₄), filtered and concentrated in vacuo. The resulting bis-azide was used directly in the next step without purification.

Crude bis-azide from the previous step (1.0 eq.) was dissolved in THF/Et₂O/H₂O (5/1/5, 0.6 M). PPh₃ (1.0 eq.) in Et₂O (0.7 M) was then added over 1 hour using a syringe pump. The resulting solution was stirred at room temperature overnight at which time precipitate formation was observed. The reaction was diluted with H₂O (100 mL) and washed with Et₂O (3×100 mL). The aqueous layer was then basified via the addition of solid NaOH to pH=11 and extracted with DCM (3×100 mL). Combined organic layers were dried (MgSO₄), filtered and concentrated in vacuo. The crude product (TLC: DCM/MeOH 8:2 R_{f}=0.2) was purified via flash column chromatography (silica gel, DCM/MeOH/Et₃N 100:0:0 to 90:10:0 to 80:10:10) to give the desired amino azide.

### 2-(2-azidoethoxy)ethan-1-amine (4a)²⁰

Compound was prepared according to GP1.

Yield: 90%, yellow oil.

¹H NMR (500 MHz, CDCl₃): δ=3.65 (t, J=5.0 Hz, 2H, 4-H), 3.54 (t, J=5.1 Hz, 2H, 2-H), 3.38 (t, J=5.0 Hz, 2H, 5-H), 2.90 (t, J=5.1 Hz, 2H, 1-H), 2.40 (s, 2H, NH₂).

¹³C NMR (125 MHz, CDCl₃): δ=72.7, 70.0, 50.7, 41.6.

### 2-(2-(2-azidoethoxy)ethoxy)ethan-1-amine (4b)²¹

Compound was prepared according to GP1.

Yield: 90%, yellow oil.

¹H NMR (500 MHz, CDCl₃): δ=3.68-3.63 (m, 6H, 4-H, 5-H, 8-H), 3.53 (t, J=5.0 Hz, 2H, 2-H), 3.39 (t, J=5.2 Hz, 2H, 6-H), 2.88 (t, 5.0 Hz, 2H, 1-H), 2.17 (s, 2H, NH₂).

¹³C NMR (125 MHz, CDCl₃): δ=73.0, 70.7, 70.3, 70.1, 50.7, 41.6.

### 2-(2-(2-(2-azidoethoxy)ethoxy)ethoxy)ethan-1-amine (4)²²

Compound was prepared according to GP1.

Yield: 85%, yellow oil.

¹H NMR (500 MHz, CDCl₃): δ=3.68-3.60 (m, 10H, 3-H, 4-H, 5-H, 6-H, 7-H), 3.50 (t, J=5.1 Hz, 2H, 2-H), 3.39 (t, J=5.1 Hz, 2H, 8-H), 2.86 (t, J=5.1 Hz, 2H, 1-H), 1.89 (s, 2H, NH₂)

¹³C NMR (125 MHz, CDCl₃): δ=73.1, 70.71, 70.66, 70.6, 70.3, 70.1, 50.7, 41.7.

### 14-azido-3,6,9,12-tetraoxatetradecan-1-amine (4d)²³

Compound was prepared according to GP1.

Yield: 73%, yellow oil.

¹H NMR (500 MHz, CDCl₃): δ=3.72-3.60 (m, 14H, 3-H, 4-H, 5-H, 6-H, 7-H, 8-H, 9-H), 3.55 (t, J=3.0 Hz, 2H, 2-H), 3.38 (t, J=4.7 Hz, 2H, 10-H), 2.90 (t, J=4.7 Hz, 2H, 1-H).

¹³C NMR (125 MHz, CDCl₃): δ=71.9, 70.7-70.0 (wide peak), 50.7, 41.4.

### 17-azido-3,6,9,12,15-pentaoxaheptadecan-1-amine (4e)²⁴

Compound was prepared according to GP1.

Yield: 70%, yellow oil.

¹H NMR (500 MHz, CDCl₃): δ=3.96 (t, J=3.2 Hz, 2H, 11-H), 3.62-3.80 (m, 18H, 2-H, 3-H, 4-H, 5-H, 6-H, 7-H, 8-H, 9-H, 10-H), 3.50 (t, J=4.90 Hz, 2H, 12-H), 3.15 (t, J=4.90 Hz, 2H, 1-H).

¹³C NMR (125 MHz, CDCl₃): δ=70.6-69.8 (wide peak), 66.9, 50.7, 40.6.

### (2R,3R)-5,7-dihydroxy-2-(3,4,5-trihydroxyphenyl)chroman-3-yl 3,5-dihydroxy-4-(prop-2-yn-1-yloxy)benzoate and (2R,3R)-2-(3,5-dihydroxy-4-(prop-2-yn-1-yloxy)phenyl)-5,7-dihydroxychroman-3-yl 3,5-dihydroxy-4-(prop-2-yn-1-yloxy)benzoate (3 and 3′) (using NaH/DMF)

To NaH (15.7 mg, 0.65 mmol, 1.5 eq.) at 0° C. was added a solution of EGCG (200 mg, 0.44 mmol, 1.0 eq.) in dry DMF (1.45 mL, 0.3 M). The resulting mixture was stirred at room temperature for 30 minutes. Propargyl bromide (53 μL, 0.48 mmol, 1.1 eq., 80% w/w) was then added and the reaction was heated to 80° C. and stirred overnight. Upon cooling to room temperature, the reaction was concentrated in vacuo and subjected to flash column chromatography (silica gel, CHCl₃/MeOH 100:0 to 15:1 to 13:1 to 11:1). Desired monopropargylated product (TLC: CHCl₃/MeOH 8:2, R_{f}=0.3) was obtained in 33% yield (72 mg) (decomposes at >120° C.), [α]_{D}²¹=−162.0° (c=0.1, MeOH) along with 15% of bispropargylated product (R_{f}=0.6), [α]_{D}²¹=−133.0° (c=0.1, MeOH) as white solids.

### Monopropargylated Product (3):

FT-IR (neat): 3358, 3290, 2124, 1697, 1606, 1522, 1454, 1371, 1347, 1242, 1196, 1147, 1056, 1039, 1017, 826, 769, 640 cm⁻¹.

¹H NMR (500 MHz, CD₃OD): δ=6.89 (s, 2H, Gal H-2, H-6), 6.47 (s, 2H, H-2′, H-6′), 5.93 (s, 2H, H-6, H-8), 5.52 (m, 1H, H-3), 4.95 (s, 1H, H-2), 4.76 (d, J=2.4 Hz, 2H, OCH₂R), 2.99-2.94 (dd, J=17.3, 4.5 Hz, 1H, H-4a), 2.85-2.80 (dd, J=17.3, 2.3 Hz, 1H, H-40), 2.77 (t, J=2.4 Hz, 1H, ═CH)

¹³C NMR (125 MHz, CD₃OD): δ=165.7, 156.5, 156.4, 155.8, 150.5, 145.3, 137.0, 132.4, 129.3, 125.7, 108.7, 105.4, 97.9, 95.2, 94.5, 78.6, 77.1, 75.3, 68.9, 58.6, 25.4

HRMS (ESI): m/z calcd for C₂₅H₂₀O₁₁ [M+H]⁺: 497.1039; found: 497.1102.

### Bispropargylated Produce (3′):

FT-IR (neat): 3359, 3282, 2926, 2858, 2362, 2124, 1695, 1601, 1519, 1451, 1363, 1235, 1174, 1142, 1049, 1014, 982, 754, 736, 711, 632 cm⁻¹.

¹H NMR (500 MHz, CD₃OD): δ=6.88 (s, 2H, Gal H-2, H-6), 6.50 (s, 2H, H-2′, H-6′), 5.94 (s, 2H, H-6, H-8), 5.55 (m, 1H, H-3), 4.99 (s, 1H, H-2), 4.76 (d, J=2.4 Hz, 2H, OCH₂R), 4.66 (d, J=2.4 Hz, 2H, OCH₂R′), 3.01-2.95 (dd, J=17.4, 4.6 Hz, 1H, H-4a), 2.86-2.81 (dd, J=17.3, 2.2 Hz, 1H, H-4), 2.77 (t, J=2.4 Hz, 1H, ═CH), 2.71 (t, J=2.4 Hz, 1H, ≡CH′)

¹³C NMR (125 MHz, CD₃OD): δ=165.6, 156.6, 156.5, 155.6, 150.5, 150.4, 137.0, 134.8, 132.3, 125.6, 108.7, 105.5, 97.9, 95.2, 94.5, 79.0, 78.6, 78.1, 75.3, 75.0, 68.8, 58.8, 58.6, 26.4

HRMS (ESI): m/z calcd for C₂₈H₂₃O₁₁ [M+H]⁺: 535.1240; found: 535.1252.

### (2R,3R)-5,7-dihydroxy-2-(3,4,5-trihydroxyphenyl)chroman-3-yl 3,5-hydroxy-4-(prop-2-yn-1-yloxy)benzoate (3) (using K₂CO₃/DMF)

To EGCG (1 g, 2.18 mmol, 1.0 eq.) in DMF (11 mL, 0.2 M) at 0° C. was added K2C03 (166 mg, 1.2 mmol, 0.5 eq.) in one portion. The reaction was stirred at room temperature for 1 hour. Propargyl bromide (0.24 mL, 2.18 mmol, 1.1 eq., 80% w/w) was then added and the reaction was stirred at the same temperature overnight. The mixture was then concentrated in vacuo and purified as above furnishing the product in 45% yield (491 mg) along with 10% of bispropargylated side product. All the analytical data matched the one obtained using an alternative procedure.

Click Reaction with PEGylated Linkers; General Procedure (GP2).

To a flame-dried microwave vial was added 3 (40 mg, 0.081 mmol, 1.0 eq) and the corresponding azide (0.081 mmol, 1.0 eq.). In a separate vial, a solution of CuSO₄·5H₂O (4 mg, 0.016 mmol, 0.2 eq.), sodium ascorbate (34 mg, 0.17 mmol. 2.0 eq.), TBTA (21 mg, 0.04 mmol, 0.5 eq.) in DMSO/H₂O (4:1, 0.81 mL, 0.1 M) was prepared and added to the first flask. The reaction was stirred at room temperature for 1 h. The crude mixture was purified by preparative reverse-phase HPLC (33-60% MeCN/H₂O+0.1% (v/v) TFA in 8.5 min) to give the title compounds (t_{R}=5.2 min).

### (2R,3R)-5,7-dihydroxy-2-(3,4,5-trihydroxyphenyl)chroman-3-yl 4-((1-(2-(2-aminoethoxy)ethyl)-H-1,2,3-triazol-4-yl)methoxy)-3,5-dihydroxybenzoate (5a)

Compound was prepared according to GP2.

Yield: 35 mg, 68%, white solid. [α]_{D}²¹=−81° (c=0.1, MeOH).

FT-IR (neat): 3374, 2951, 2934, 1676, 1626, 1523, 1448, 1370, 1196, 1146, 1061, 1015, 770, 724, 650, 612 cm⁻¹

¹H NMR (500 MHz, CD₃OD): δ=7.88 (s, 1H, triazole-H), 6.87 (s, 2H, Gal H-2, H-6), 6.50 (s, 2H, H-2′, H-6′), 5.95 (s, 2H, H-6, H-8), 5.54 (m, 1H, H-3), 5.25 (s, 2H, OCH₂R), 4.97 (s, 1H, H-2),

4.56 (t, J=4.8 Hz, 2H, N—CH₂CH₂OCH₂CH₂NH₂),

3.83-3.75 (m, 2H, N—CH₂CH₂OCH₂CH₂NH₂).

3.50-3.48 (m, 2H, N—CH₂CH₂OCH₂CH₂NH₂),

3.01-2.97 (m, 3H, N—CH₂CH₂OCH₂CH₂NH₂, and H-4α),

2.86-2.82 (dd, J=17.3, 2.2 Hz, 1H, H-4β).

¹³C NMR (125 MHz, CD₃OD): δ=165.6, 156.5, 156.5, 155.8, 150.4, 145.3, 137.1, 132.3, 129.4, 125.7, 124.9, 108.8, 105.4, 97.8, 95.1, 94.5, 77.0, 69.1, 69.0, 66.3, 63.9, 49.9, 39.0, 25.4

HRMS (ESI): m/z calcd for C₂₉H₃₂N₄O₂ [M+H]⁺: 627.1938; found: 627.1954.

### (2R,3R)-5,7-dihydroxy-2-(3,4,5-trihydroxyphenyl)chroman-3-yl 4-((1-(2-(2-aminoethoxy)ethoxy)ethyl)-1H-1,2,3-triazol-4-yl)methoxy)-3,5-dihydroxybenzoate (5b)

Compound was prepared according to GP2.

Yield: 32 mg, 58%, white solid. [α]_{D}²¹=−73° (c=0.1, MeOH).

FT-IR (neat): 3170, 2964, 2952, 1678, 1627, 1609, 1523, 1450, 1376, 1347, 1201, 1146, 1058, 1039, 969, 836, 720, 677, 602 cm⁻¹.

¹H NMR (500 MHz, CD₃OD): 5=7.90 (s, 1H, triazole-H), 6.88 (s, 2H, Gal H-2, H-6), 6.50 (s, 2H, H-2′, H-6′), 5.95 (s, 2H, H-6, H-8), 5.53-5.52 (m, 1H, H-3), 5.26 (s, 2H, OCH₂R), 4.97 (s, 1H, H-2),

4.52-4.50 (t, J=4.6 Hz, 2H, N—CH₂CH₂OCH₂CH₂OCH₂CH₂NH₂),

3.82-3.73 (m, 2H, N—CH₂CH₂OCH₂CH₂OCH₂CH₂NH₂),

3.50-3.48 (m, 2H, N—CH₂CH₂OCH₂CH₂OCH₂CH₂NH₂),

3.41 (m, 4H, N—CH₂CH₂OCH₂CH₂OCH₂CH₂NH₂),

3.02-2.96 (m, 3H, N—CH₂CH₂OCH₂CH₂OCH₂CH₂NH₂, and H-4α),

2.86-2.82 (dd, J=18.1, 1.9 Hz, 1H, H-4β).

¹³C NMR (125 MHz, CD₃OD): δ=165.6, 156.54, 156.46, 155.8, 150.4, 145.3, 137.1, 132.3, 129.4, 125.6, 108.9, 105.3, 97.8, 95.1, 94.5, 77.0, 70.1, 69.8, 68.98, 68.95, 66.3, 63.8, 53.7, 50.1, 39.3, 25.5

HRMS (ESI): m/z calcd for C₃₁H₃₅N₄O₁₃ [M+H]⁺: 671.2201; found: 671.2170 (2R,3R)-5,7-dihydroxy-2-(3,4,5-trihydroxyphenyl)chroman-3-yl 4-((1-(2-(2-(2-(2-aminoethoxy)ethoxy)ethoxy)ethyl)-H-1,2,3-triazol-4-yl)methoxy)-3,5-dihydroxybenzoate (5c)

Compound was prepared according to GP2.

Yield: 30 mg, 52%, white solid. [α]_{D}²¹=−62° (c=0.1. MeOH).

FT-IR (neat): 3203, 2970, 1674, 1602, 1523, 1437, 1368, 1200, 1143, 1060, 981, 831, 718, 647, 622, 610 cm⁻¹.

¹H NMR (500 MHz, CD₃OD): δ=7.87 (s, 1H, triazole-H), 6.89 (s, 2H, Gal H-2, H-6), 6.50 (s, 2H, H-2′, H-6′), 5.94 (s, 2H, H-6, H-8), 5.54-5.53 (m, 1H, H-3), 5.25 (s, 2H, OCH₂R), 4.97 (s, 1H, H-2),

4.52 (t, J=4.9 Hz, 2H, N—CH₂CH₂(OCH₂CH₂)₂OCH₂CH₂NH₂),

3.81-3.73 (m, 2H, N—CH₂CH₂(OCH₂CH₂)₂OCH₂CH₂NH₂),

3.59-3.57 (m, 2H, N—CH₂CH₂(OCH₂CH₂)₂OCH₂CH₂NH₂),

3.54-3.47 (m, 4H, N—CH₂CH₂(OCH₂CH₂)₂OCH₂CH₂NH₂,

3.45-3.42 (m, 4H, N—CH₂CH₂(OCH₂CH₂)₂OCH₂CH₂NH₂),

3.07-3.05 (t, J=5.5 Hz, 2H, N—CH₂CH₂(OCH₂CH₂)₂OCH₂CH₂NH₂), 3.01-2.96 (dd, J=17.3, 4.5 Hz, 1H, H-4α), 2.86-2.82 (dd, J=17.3, 2.3 Hz, 1H, H-4β)

¹³C NMR (125 MHz, CD₃OD): δ=165.6, 156.54, 156.48, 155, 8, 150.4, 145.3, 137.0, 132.3, 129.4, 125.6, 124.9, 108.9, 105.3, 97.8, 95.1, 94.5, 77.0, 70.0, 69.9, 69.6, 69.0, 66.3, 63.9, 50.1, 39.3, 25.5

HRMS (ESI): m/z calcd for C₃₃H₃₈N₄O₁₄Na [M+Na]⁺: 737.2282; found: 737.2308

### (2R,3R)-5,7-dihydroxy-2-(3,4,5-trihydroxyphenyl)chroman-3-yl 4-((1-(14-amino-3,6,9,12-tetraoxatetradecyl)-1H-1,2,3-triazol-4-yl)methoxy)-3,5-dihydroxybenzoate (5d)

Compound was prepared according to GP2.

Yield: 28 mg, 46%, white solid. [α]_{D}²¹=−69° (c=0.1, MeOH).

FT-IR (neat): 3179, 2926, 1681, 1627, 1523, 1451, 1372, 1349, 1203, 1146, 1061, 1038, 831, 726, 641, 618 cm⁻¹.

¹H NMR (500 MHz, CD₃OD): δ=7.90 (s, 1H, triazole-H), 6.87 (s, 2H, Gal H-2, H-6), 6.48 (s, 2H, H-2′, H-6′), 5.93 (s, 2H, H-6, H-8), 5.52-5.53 (m, 1H, H-3), 5.23 (s, 2H, OCH₂R), 4.95 (s, 1H, H-2),

4.51-4.48 (t, J=4.7 Hz, 2H, N—CH₂CH₂(OCH₂CH₂)₃OCH₂CH₂NH₂),

3.78-3.70 (m, 2H, N—CH₂CH₂(OCH₂CH₂)₃OCH₂CH₂NH₂),

3.61-3.58 (m, 2H, N—CH₂CH₂(OCH₂CH₂)₃OCH₂CH₂NH₂),

3.55-3.50 (m, 6H, N—CH₂CH₂(OCH₂CH₂)₃OCH₂CH₂NH₂),

3.46-3.40 (m, 6H, N—CH₂CH₂(OCH₂CH₂)₃OCH₂CH₂NH₂), 3.05-3.03 (t, J=5.0 Hz, 2H, N—CH₂CH₂(OCH₂CH₂)₃OCH₂CH₂NH₂, 3.00-2.94 (dd, J=17.5, 4.6 Hz, 1H, H-4α), 2.85-2.80 (dd, J=17.5, 1.8 Hz, 1H, H-4β)

¹³C NMR (125 MHz, CD₃OD): δ=165.6, 156.50, 156.45, 155.8, 150.4, 145.3, 137.2, 132.3, 129.4, 125.6, 125.0, 108.9, 105.3, 97.8, 95.1, 94.5, 77.0, 70.05, 69.94, 69.88, 69.82, 69.78, 69.5, 69.0, 68.9, 66.3, 64.0, 50.1, 39.2, 25.5

HRMS (ESI): m/z calcd for C35H42N₄O₁₅Na [M+Na]*: 781.2544; found: 781.2525 (2R,3R)-5,7-dihydroxy-2-(3,4,5-trihydroxyphenyl)chroman-3-yl 4-((1-(17-amino-3,6,9,12,15-pentaoxaheptadecyl)-H-1,2,3-triazol-4-yl)methoxy)-3,5-dihydroxybenzoate (5e)

Compound was prepared according to GP2.

Yield: 29 mg, 45/a, white solid. [α]_{D}²¹=−79° (c=0.1, MeOH).

FT-IR (neat): 3307, 2908, 1678, 1625, 1521, 1449, 1374, 1349, 1238, 1201, 1147, 1096, 845, 772, 722, 652, 633 cm⁻¹.

¹H NMR (500 MHz, CD₃OD): δ=7.90 (s, 1H, triazole-H), 6.87 (s, 2H, Gal H-2, H-6), 6.49 (s, 2H, H-2′, H-6′), 5.93 (s, 2H, H-6, H-8), 5.52-5.53 (m, 1H, H-3), 5.24 (s, 2H, OCH₂R), 4.95 (s, 1H, H-2),

4.52-4.50 (t, J=4.7 Hz, 2H, N—CH₂CH₂(OCH₂CH₂)₄OCH₂CH₂NH₂),

3.82-3.71 (m, 2H, N—CH₂CH₂(OCH₂CH₂)₄OCH₂CH₂NH₂),

3.63-3.60 (m, 2H, N—CH₂CH₂(OCH₂CH₂)₄OCH—CH₂NH₂),

3.57-3.38 (m, 16H, N—CH₂CH₂(OCH₂CH₂)₄OCH₂CH₂NH₂,

3.01-2.94 (m, 3H, N—CH₂CH₂(OCH₂CH₂)₄OCH₂CH₂NH₂, H-4α), 2.85-2.80 (dd, J=17.5, 1.8 Hz, 1H, H-4β)

¹³C NMR (125 MHz, CD₃OD): δ=165.6, 156.56, 156.49, 155.8, 150.4, 145.3, 143.6, 137.2, 132.3, 129.4, 125.6, 124.9, 108.9, 105.3, 97.8, 95.1, 94.5, 77.0, 69.94, 69.87, 69.84, 69.80, 69.76, 69.70, 69.68, 69.43, 69.0, 68.9, 66.3, 63.9, 50.0, 39.2, 25.5

HRMS (ESI): m/z calcd for C37H47N₄O₁₆ [M+H]⁺: 803.2987; found: 803.2997.

### REFERENCES FOR EXAMPLE 5

(1) Present Address: Department of Pharmacology and Pharmaceutical Sciences, University of Southern California School of Pharmacy, 1985 Zonal Ave, Los Angeles Calif. 90089-9121
(2) Alzheimer's & Dementia 2020, 16, 391.
(3) Gouras, G. K.; Olsson, T. T.; Hansson, O. Neurotherapeutics 2015, 12, 3.
(4) Murray, M. E.; Lowe, V. J.; Graff-Radford, N. R.; Liesinger, A. M.; Cannon, A.; Przybelski, S. A.: Rawal, B.; Parisi, J. E.; Petersen, R. C.; Kantarci, K.; Ross, O. A.; Duara, R.; Knopman, D. S.; Jack, C. R.; Dickson, D. W. Brain 2015, 138, 1370.
(5) (a) Bieschke, J.; Russ, J.; Friedrich, R. P.; Ehrnhoefer, D. E.; Wobst, H.; Neugebauer, K.; Wanker, E. E. Proceedings of the National Academy of Sciences of the United States of America 2010, 107, 7710. (b) Debnath, K.; Shekhar, S.; Kumar, V.; Jana. N. R.; Jana. N. R. ACS Applied Materials and Interfaces 2016, 8, 20309.
(6) Wobst, H. J.; Sharma, A.; Diamond, M. I.; Wanker, E. E.; Bieschke, J. FEBS Letters 2015, 589, 77.
(7) (a) Seidler, P. M.; Boyer. D. R., Sawaya, M. R.; Ge, P.; Shin, W. S.; DeTure, M. A.; Dickson, D. W.; Jiang, L.; Eisenberg, D. S. bioRxiv 2020, 2020.05.29.124537. (b) Sonawane, S. K.; Chidambaram, H.; Boral, D.; Gorantla, N. V.; Balmik, A. A.; Dangi, A.; Ramasamy. S.; Marelli, U. K.; Chinnathambi, S. Scientific Reports 2020, 10.
(8) (a) Lu, M.; Cohen, M. H.; Rieves, D.; Pazdur, R. American Journal of Hematology 2010, 85, 315. (b) Muldoon, L. L.; S??ndor, M.; Pinkston, K. E.; Neuwelt, E. A. Neurosurgery 2005, 57, 785. (c) Neubert, J.; Wagner, S.; Kiwit, J.; Brauer, A. U.; Glumm, J. International Journal of Nanomedicine 2015, 10, 2033. (d) Nguyen, K. L.; Yoshida, T.; Kathuria-Prakash, N.; Zaki, I. H.; Varallyay, C. G.: Semple, S. I.; Saouaf, R.; Rigsby, C. K.; Stoumpos, S.; Whitehead, K. K.; Griffin, L. M.; Saloner, D.; Hope, M. D.; Prince, M. R.; Fogel, M. A.: Schiebler, M. L.; Roditi, G. H.: Radjenovic. A.; Newby, D. E.; Neuwelt. E. A.; Bashir, M. R.; Hu, P.; Paul Finn. J. Radiology 2019, 293, 554.
(9) Wang, J.; Sun, P., Wang, Q.; Zhang, P.; Wang, Y.; Zi, C.; Wang, X.; Sheng, J. Cancer Cell International 2019, 19.
(10) Yang, Q.; Sheng, M.; Henkelis, J. J.; Tu, S.; Wiensch, E.; Zhang, H.; Zhang, Y.; Tucker, C.; Ejeh, D. E. Organic Process Research and Development 2019, 23, 2210.
(11) (a) Aufort, M.; Herscovici, J.; Bouhours. P.; Moreau, N.; Girard, C. Bioorganic and Medicinal Chemistry Letters 2008, 18, 1195. (b) Dieterich, D. C.: Lee, J. J.; Link, A. J.; Graumann, J.; Tirrell, D. A.; Schuman, E. M. Nature Protocols 2007, 2, 532. (c) Flows, M. C.; Ledo, A. L.; Narine, S. S. BioMed Research International 2014, 2014. (d) Lee, W.; Sarkar, S.; Pal, R.; Kim, J. Y.: Park, H.; Huynh, P. T.: Bhise, A.; Bobba, K. N.; Kim, K. I.; Ha, Y. S.; Soni, N.; Kim, W.; Lee, K.; Jung, J. M.; Rajkumar, S.; Lee, K. C.: Yoo, J. ACS Applied Bio Materials 2021, 4. (e) Sun, P.; Chen, J.; Liu, J. a.; Zhang, K. Macromolecules 2017, 50, 1463.
(12) Himo, F.; Lovell, T.; Hilgraf, R.; Rostovtsev, V. V.: Noodleman, L.; Sharpless, K. B.; Fokin, V. V. Journal of the American Chemical Society 2005, 127, 210.
(13) Rostovtsev, V. V.; Green, L. G.; Fokin. V. V.; Sharpless, K. B. Angewandte Chemie—International Edition 2002, 41, 2596.
(14) Chan, T. R.; Hilgraf, R.; Sharpless, K. B.: Fokin, V. V. Organic Letters 2004, 6, 2853.
(15) Madhusudanan, K. P.; Sachdev, K.; Harrison. D. A.; Kulshreshtha, D. K. International Journal of Mass Spectrometry and Ion Processes 1984, 62, 289.
(16) Jordheim, M.; Fossen, T.; Songstad, J.; Andersen, Ø. M. Journal of Agricultural and Food Chemistry 2007, 55, 8261.
(17) (a) Clavaguera, F.; Akatsu, H.; Fraser, G., Crowther, R. A.; Frank, S.; Hench, J.; Probst, A.; Winkler, D. T.; Reichwald, J.; Staufenbiel, M.; Ghetti, B.; Goedert, M.; Tolnay, M. Proceedings of the National Academy of Sciences of the United States of America 2013, 110, 9535. (b) Hyman, B. T. Neuron 2014, 82, 1189. (c) Seidler, P. M.; Boyer, D. R.; Murray, K. A.; Yang, T. P.: Bentzel, M.; Sawaya, M. R.: Rosenberg, G.; Cascio, D.; Williams, C. K.; Newell, K. L.; Ghetti, B.; DeTure, M. A.: Dickson, D. W.; Vinters, H. V.; Eisenberg, D. S. Journal of Biological Chemistry 2019, 294, 16451.
(18) (a) Muldoon, L. L.; Sandor, M.; Pinkston, K. E.; Neuwelt, E. A. Neurosurgery 2005, 57, 785. (b) Toth, G. B.: Varallyay, C. G.: Horvath, A.; Bashir, M. R.; Choyke, P. L.; Daldrup-Link, H. E.; Dosa, E.; Finn, J. P.; Gahramanov, S.; Harisinghani, M.; Macdougall, I.; Neuwelt, A.; Vasanawala. S. S.; Ambady, P.; Barajas, R.; Cetas, J. S.; Ciporen, J.; DeLoughery, T. J.; Doolittle, N. D.; Fu, R.; Grinstead, J.; Guimaraes, A. R.; Hamilton, B. E.; Li, X.; McConnell, H. L.; Muldoon, L. L.; Nesbit, G.; Netto, J. P.; Petterson, D.; Rooney, W. D.; Schwartz, D.; Szidonya, L.; Neuwelt, E. A. Kidney International 2017, 92, 47. (c) Nguyen, K.-L.; Yoshida. T.; Kathuria-Prakash, N.; Zaki, I. H.; Varallyay, C. G.; Semple, S. I.; Saouaf, R.; Rigsby, C. K.; Stoumpos, S.; Whitehead, K. K.; Griffin, L. M.; Saloner, D.; Hope, M. D.; Prince, M. R.: Fogel, M. A.; Schiebler, M. L.; Roditi, G. H.; Radjenovic, A.; Newby. D. E.; Neuwelt, E. A.; Bashir, M. R.; Hu, P.; Finn, J. P. Radiology 2019, 293, 554. (d) Lu, M.; Cohen, M. H.; Rieves, D.; Pazdur, R. American Journal of Hematology 2010, NA. (e) Enteshari Najafabadi, R.; Kazemipour, N.; Esmacili, A.; Beheshti, S.; Nazifi, S. BMC Pharmacology and Toxicology 2018, 19.
(19) (a) He, A.; Guan, X.; Song, H.; Li, S.; Huang, K. Food Bioscience 2020, 37. (b) Radhakrishnan, R.; Kulhari, H.; Pooja, D.; Gudem, S.; Bhargava, S.; Shukla, R.; Sistla, R. Chemistry and Physics of Lipids 2016, 198, 51. (c) Sanna, V.; Singh, C. K.; Jashari, R.; Adhami, V. M.; Chamcheu, J. C.; Rady, I.; Sechi, M.; Mukhtar, H.; Siddiqui, I. A. Scientific Reports 2017, 7. (d) Smith, A.; Giunta. B.; Bickford, P. C.; Fountain. M.; Tan, J.; Shytle, R. D. International Journal of Pharmaceutics 2010, 389, 207. (e) Zou, L. q.; Peng, S. f.; Liu, W.; Gan, L.; Liu, W. I.; Liang, R. h.: Liu, C. m.; Niu, J.; Cao, Y. I.: Liu, Z.; Chen, X. Food Research International 2014, 64, 492.
(20) Tran, F.; Odell, A. V.; Ward, G. E.; Westwood. N. J. Molecules 2013, 18, 11639.
(21) Friscourt, F.; Fahmi, C. J.; Boons, G. J. Journal of the American Chemical Society 2012, 134, 18809.
(22) Bertozzi, C. R.; Bednarski, M. D. Journal of Organic Chemistry 1991, 56, 4326.
(23) Brauch, S.; Henze, M.; Osswald, B.; Naumann, K.; Wessjohann, L. A.; Van Berkel, S. S.; Westermann, B. Organic and Biomolecular Chemistry 2012, 10, 958.
(24) Wang, H.; Raghupathi, K. R.; Zhuang, J.; Thayumanavan, S. ACS Macro Letters 2015, 4, 422.

### Example 6: Additional Compounds

The following are a series of tables that have additional analogs that can be readily synthesized using methods described herein, for example in Example 5.

Dimeric analogs

| | |
|---|---|
| 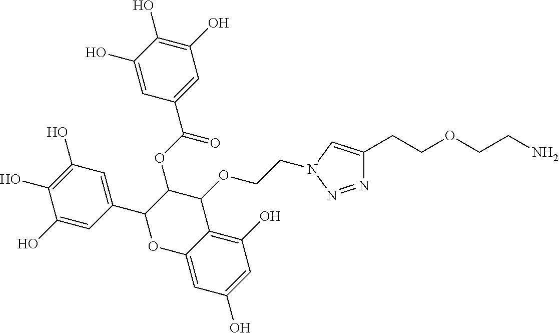 [SMILES: NCCOCCc1cn(CCOC2c3c(O)cc(O)cc3OC(c3cc(O)c(O)c(O)c3)C2OC(=O)c2cc(O)c(O)c(O)c2)nn1] | 15 |
| 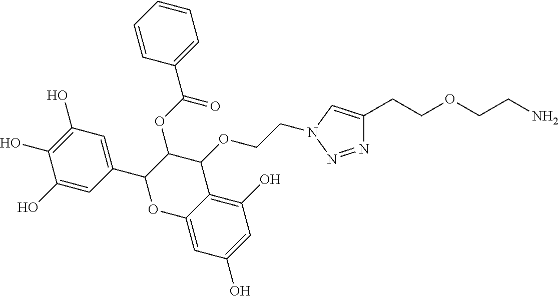 [SMILES: NCCOCCc1cn(CCOC2c3c(O)cc(O)cc3OC(c3cc(O)c(O)c(O)c3)C2OC(=O)c2ccccc2)nn1] | 16 |
| 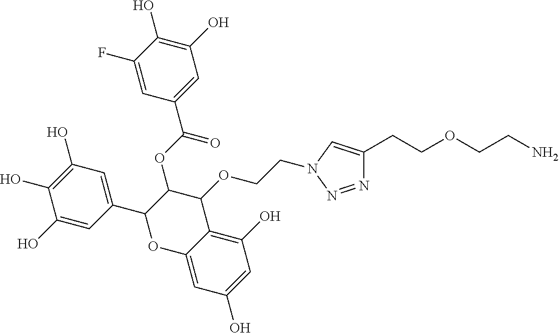 [SMILES: NCCOCCc1cn(CCOC2c3c(O)cc(O)cc3OC(c3cc(O)c(O)c(O)c3)C2OC(=O)c2cc(O)c(O)c(F)c2)nn1] | 17 |
| 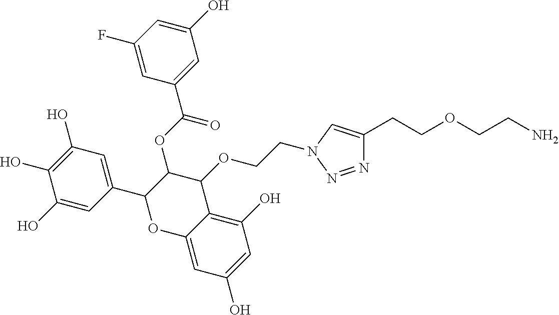 [SMILES: NCCOCCc1cn(CCOC2c3c(O)cc(O)cc3OC(c3cc(O)c(O)c(O)c3)C2OC(=O)c2cc(O)cc(F)c2)nn1] | 18 |
| 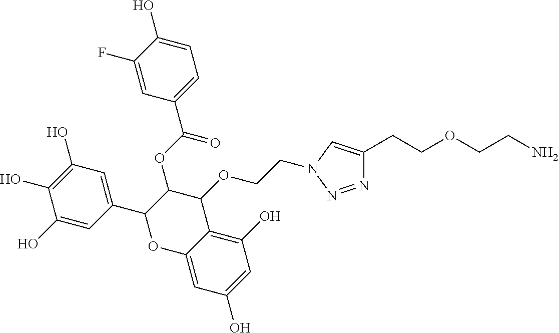 [SMILES: NCCOCCc1cn(CCOC2c3c(O)cc(O)cc3OC(c3cc(O)c(O)c(O)c3)C2OC(=O)c2ccc(O)c(F)c2)nn1] | 19 |
| 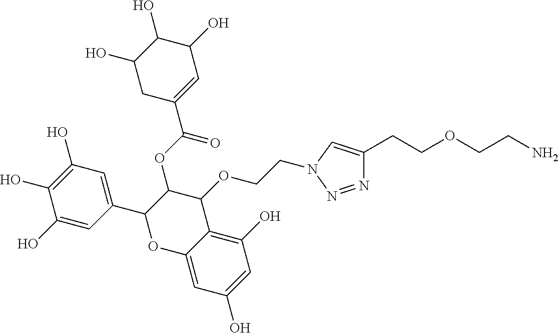 [SMILES: NCCOCCc1cn(CCOC2c3c(O)cc(O)cc3OC(c3cc(O)c(O)c(O)c3)C2OC(=O)C2=CC(O)C(O)C(O)C2)nn1] | 20 |
| 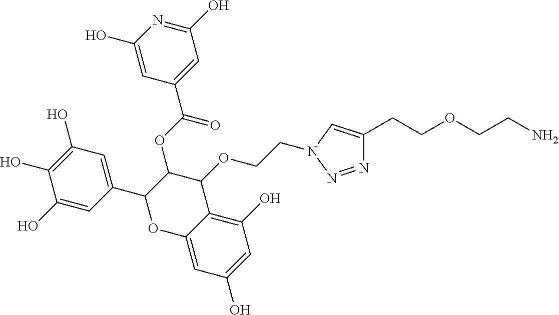 [SMILES: NCCOCCc1cn(CCOC2c3c(O)cc(O)cc3OC(c3cc(O)c(O)c(O)c3)C2OC(=O)c2cc(O)nc(O)c2)nn1] | 21 |
| 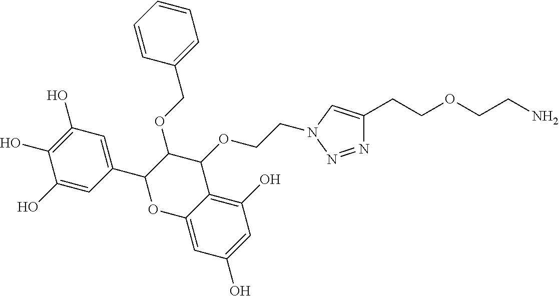 [SMILES: NCCOCCc1cn(CCOC2c3c(O)cc(O)cc3OC(c3cc(O)c(O)c(O)c3)C2OCc2ccccc2)nn1] | 22 |
| 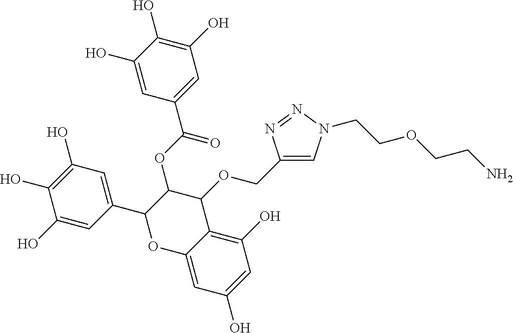 [SMILES: NCCOCCn1cc(COC2c3c(O)cc(O)cc3OC(c3cc(O)c(O)c(O)c3)C2OC(=O)c2cc(O)c(O)c(O)c2)nn1] | 23 |
| 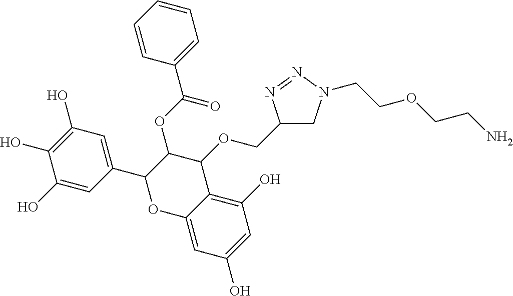 [SMILES: NCCOCCN1CC(COC2c3c(O)cc(O)cc3OC(c3cc(O)c(O)c(O)c3)C2OC(=O)c2ccccc2)N=N1] | 24 |
| 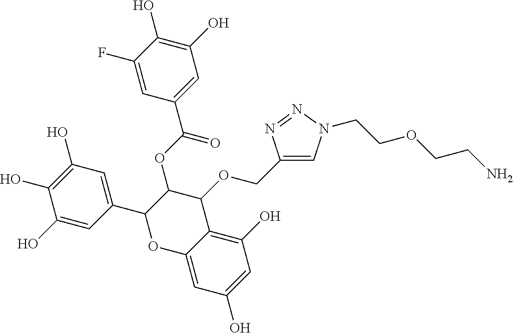 [SMILES: NCCOCCn1cc(COC2c3c(O)cc(O)cc3OC(c3cc(O)c(O)c(O)c3)C2OC(=O)c2cc(O)c(O)c(F)c2)nn1] | 25 |
| 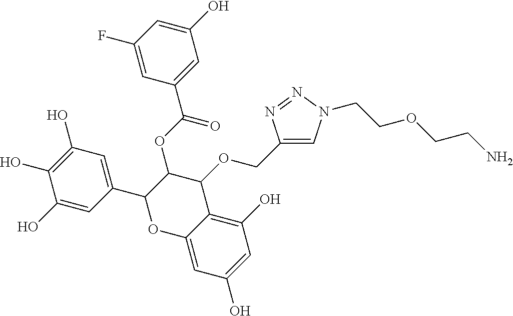 [SMILES: NCCOCCn1cc(COC2c3c(O)cc(O)cc3OC(c3cc(O)c(O)c(O)c3)C2OC(=O)c2cc(O)cc(F)c2)nn1] | 26 |
| 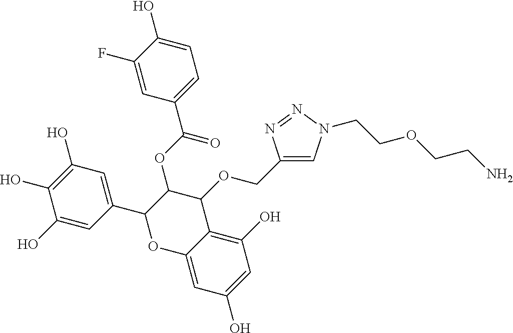 [SMILES: NCCOCCn1cc(COC2c3c(O)cc(O)cc3OC(c3cc(O)c(O)c(O)c3)C2OC(=O)c2ccc(O)c(F)c2)nn1] | 27 |
| 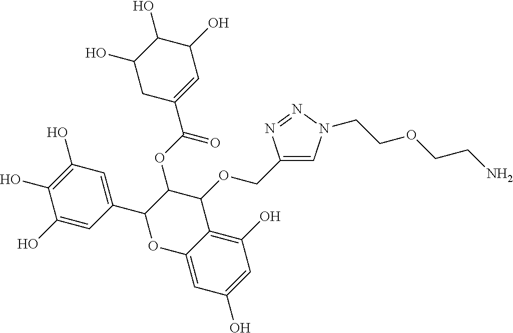 [SMILES: NCCOCCn1cc(COC2c3c(O)cc(O)cc3OC(c3cc(O)c(O)c(O)c3)C2OC(=O)C2=CC(O)C(O)C(O)C2)nn1] | 28 |
| 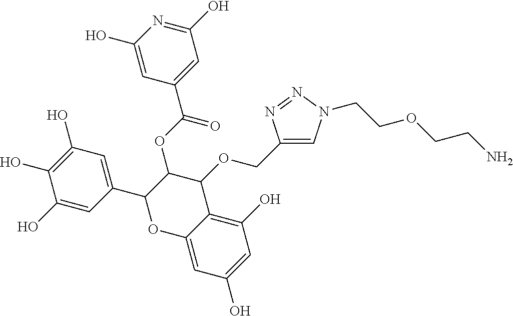 [SMILES: NCCOCCn1cc(COC2c3c(O)cc(O)cc3OC(c3cc(O)c(O)c(O)c3)C2OC(=O)c2cc(O)nc(O)c2)nn1] | 29 |
| 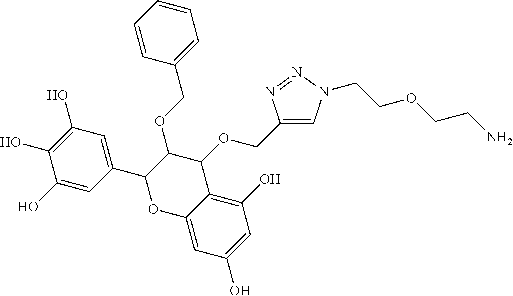 [SMILES: NCCOCCn1cc(COC2c3c(O)cc(O)cc3OC(c3cc(O)c(O)c(O)c3)C2OCc2ccccc2)nn1] | 30 |
| 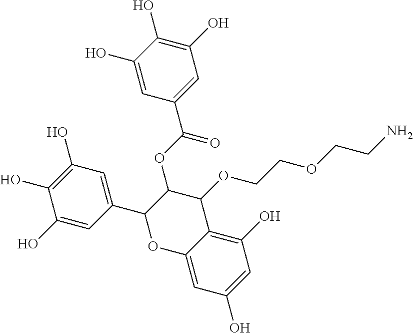 [SMILES: NCCOCCOC1c2c(O)cc(O)cc2OC(c2cc(O)c(O)c(O)c2)C1OC(=O)c1cc(O)c(O)c(O)c1] | 31 |
| 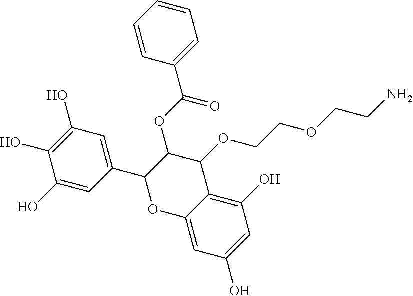 [SMILES: NCCOCCOC1c2c(O)cc(O)cc2OC(c2cc(O)c(O)c(O)c2)C1OC(=O)c1ccccc1] | 32 |
| 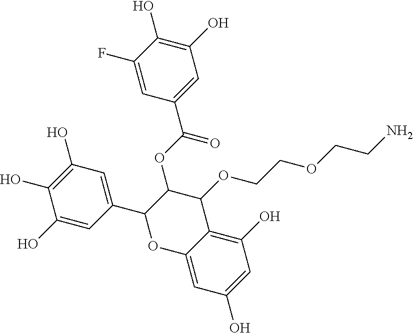 [SMILES: NCCOCCOC1c2c(O)cc(O)cc2OC(c2cc(O)c(O)c(O)c2)C1OC(=O)c1cc(O)c(O)c(F)c1] | 33 |
| 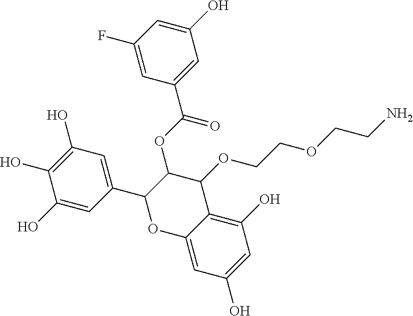 [SMILES: NCCOCCOC1c2c(O)cc(O)cc2OC(c2cc(O)c(O)c(O)c2)C1OC(=O)c1cc(O)cc(F)c1] | 34 |
| 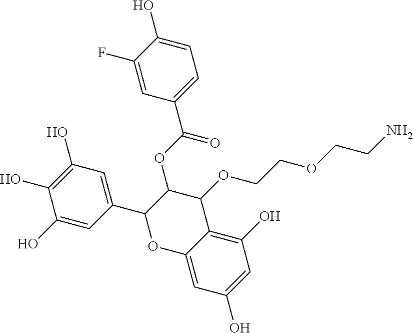 [SMILES: NCCOCCOC1c2c(O)cc(O)cc2OC(c2cc(O)c(O)c(O)c2)C1OC(=O)c1ccc(O)c(F)c1] | 35 |
| 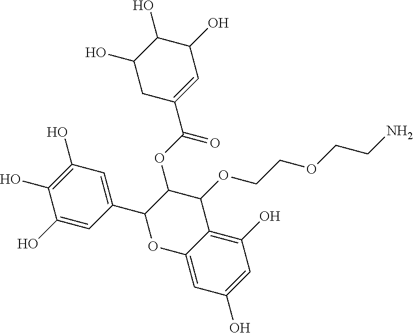 [SMILES: NCCOCCOC1c2c(O)cc(O)cc2OC(c2cc(O)c(O)c(O)c2)C1OC(=O)C1=CC(O)C(O)C(O)C1] | 36 |
| 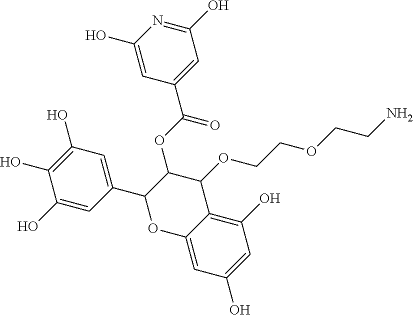 [SMILES: NCCOCCOC1c2c(O)cc(O)cc2OC(c2cc(O)c(O)c(O)c2)C1OC(=O)c1cc(O)nc(O)c1] | 37 |
| 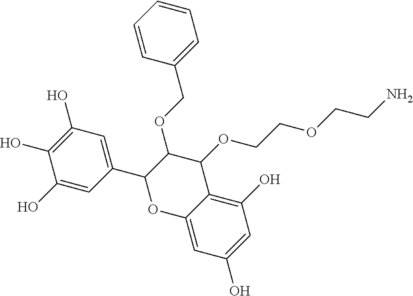 [SMILES: NCCOCCOC1c2c(O)cc(O)cc2OC(c2cc(O)c(O)c(O)c2)C1OCc1ccccc1] | 38 |

TABLE D
Trimeric analogs

| | |
|---|---|
| 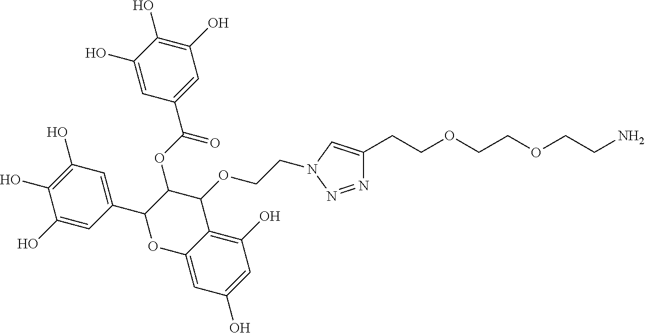 [SMILES: NCCOCCOCCc1cn(CCOC2c3c(O)cc(O)cc3OC(c3cc(O)c(O)c(O)c3)C2OC(=O)c2cc(O)c(O)c(O)c2)nn1] | 39 |
| 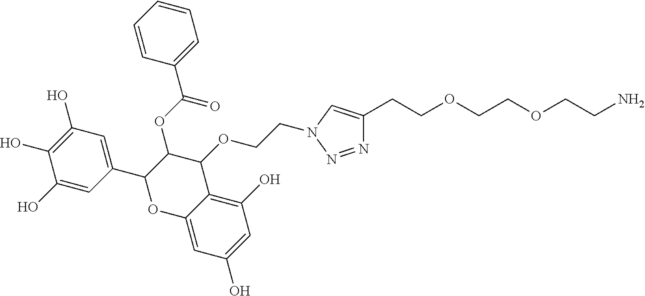 [SMILES: NCCOCCOCCc1cn(CCOC2c3c(O)cc(O)cc3OC(c3cc(O)c(O)c(O)c3)C2OC(=O)c2ccccc2)nn1] | 40 |
| 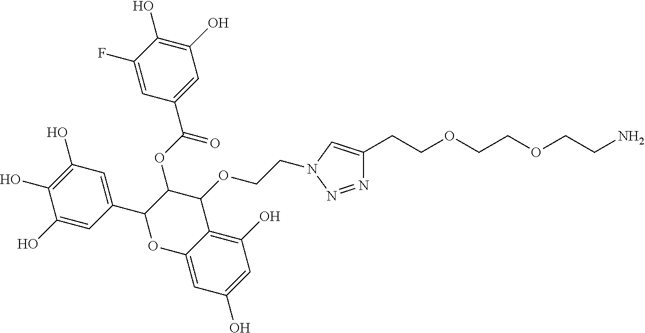 [SMILES: NCCOCCOCCc1cn(CCOC2c3c(O)cc(O)cc3OC(c3cc(O)c(O)c(O)c3)C2OC(=O)c2cc(O)c(O)c(F)c2)nn1] | 41 |
| 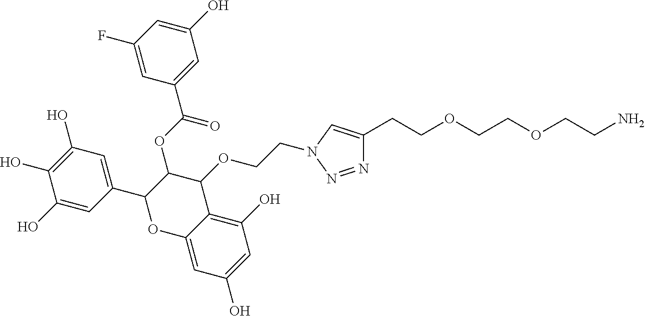 [SMILES: NCCOCCOCCc1cn(CCOC2c3c(O)cc(O)cc3OC(c3cc(O)c(O)c(O)c3)C2OC(=O)c2cc(O)cc(F)c2)nn1] | 42 |
| 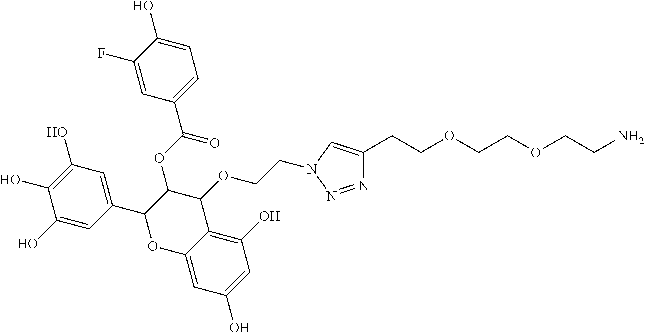 [SMILES: NCCOCCOCCc1cn(CCOC2c3c(O)cc(O)cc3OC(c3cc(O)c(O)c(O)c3)C2OC(=O)c2ccc(O)c(F)c2)nn1] | 43 |
| 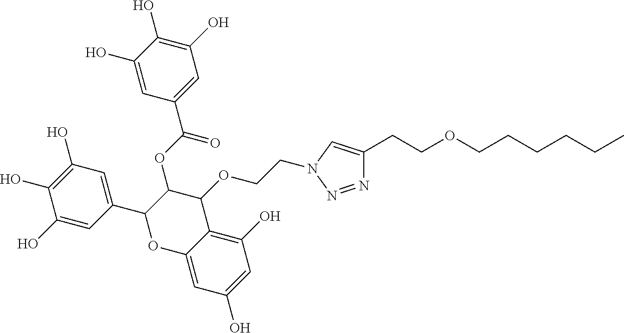 [SMILES: CCCCCCOCCc1cn(CCOC2c3c(O)cc(O)cc3OC(c3cc(O)c(O)c(O)c3)C2OC(=O)c2cc(O)c(O)c(O)c2)nn1] | 44 |
| 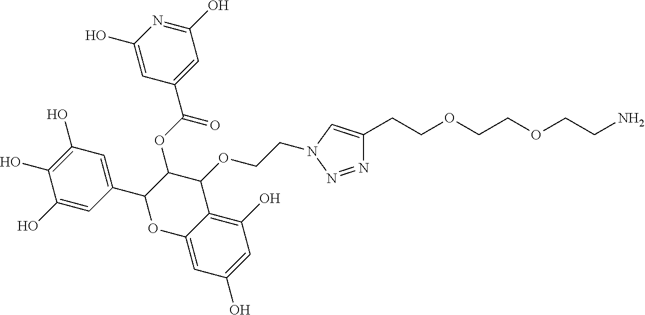 [SMILES: NCCOCCOCCc1cn(CCOC2c3c(O)cc(O)cc3OC(c3cc(O)c(O)c(O)c3)C2OC(=O)c2cc(O)nc(O)c2)nn1] | 45 |
| 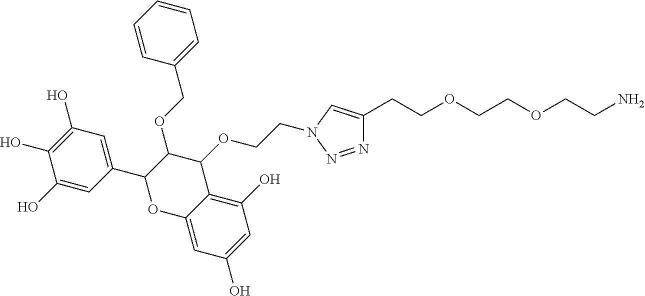 [SMILES: NCCOCCOCCc1cn(CCOC2c3c(O)cc(O)cc3OC(c3cc(O)c(O)c(O)c3)C2OCc2ccccc2)nn1] | 46 |
| 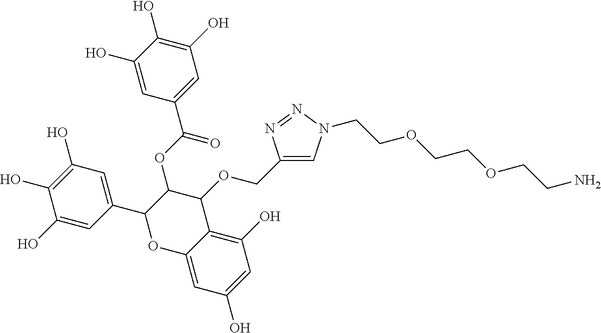 [SMILES: NCCOCCOCCn1cc(COC2c3c(O)cc(O)cc3OC(c3cc(O)c(O)c(O)c3)C2OC(=O)c2cc(O)c(O)c(O)c2)nn1] | 47 |
| 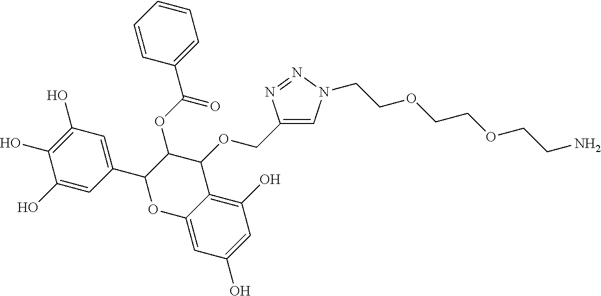 [SMILES: NCCOCCOCCn1cc(COC2c3c(O)cc(O)cc3OC(c3cc(O)c(O)c(O)c3)C2OC(=O)c2ccccc2)nn1] | 48 |
| 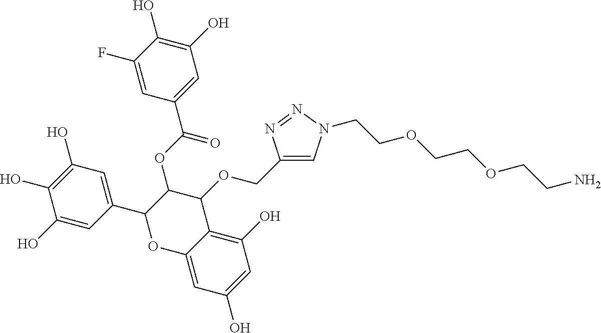 [SMILES: NCCOCCOCCn1cc(COC2c3c(O)cc(O)cc3OC(c3cc(O)c(O)c(O)c3)C2OC(=O)c2cc(O)c(O)c(F)c2)nn1] | 49 |
| 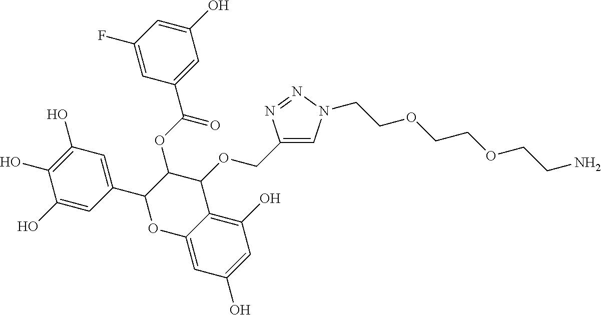 [SMILES: NCCOCCOCCn1cc(COC2c3c(O)cc(O)cc3OC(c3cc(O)c(O)c(O)c3)C2OC(=O)c2cc(O)cc(F)c2)nn1] | 50 |
| 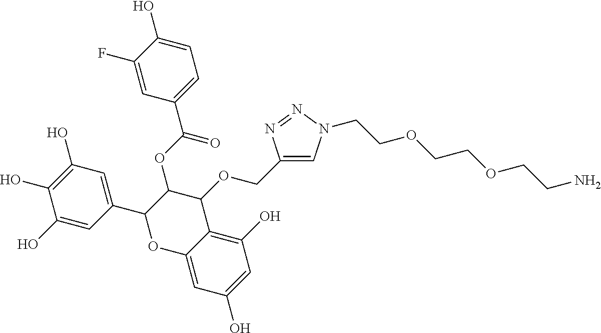 [SMILES: NCCOCCOCCn1cc(COC2c3c(O)cc(O)cc3OC(c3cc(O)c(O)c(O)c3)C2OC(=O)c2ccc(O)c(F)c2)nn1] | 51 |
| 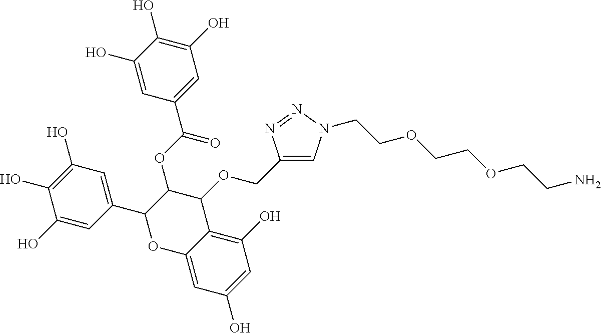 [SMILES: NCCOCCOCCn1cc(COC2c3c(O)cc(O)cc3OC(c3cc(O)c(O)c(O)c3)C2OC(=O)c2cc(O)c(O)c(O)c2)nn1] | 52 |
| 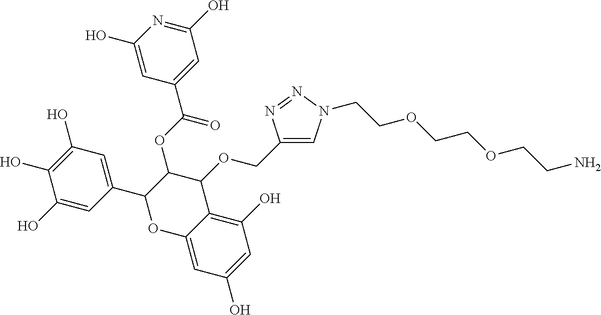 [SMILES: NCCOCCOCCn1cc(COC2c3c(O)cc(O)cc3OC(c3cc(O)c(O)c(O)c3)C2OC(=O)c2cc(O)nc(O)c2)nn1] | 53 |
| 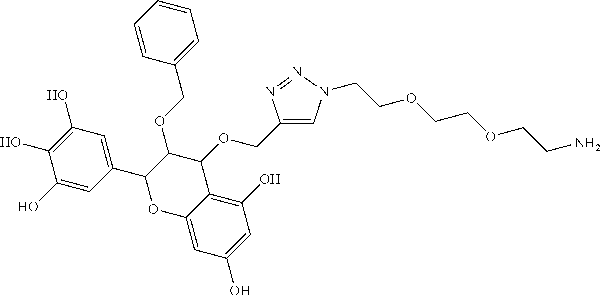 [SMILES: NCCOCCOCCn1cc(COC2c3c(O)cc(O)cc3OC(c3cc(O)c(O)c(O)c3)C2OCc2ccccc2)nn1] | 54 |
| 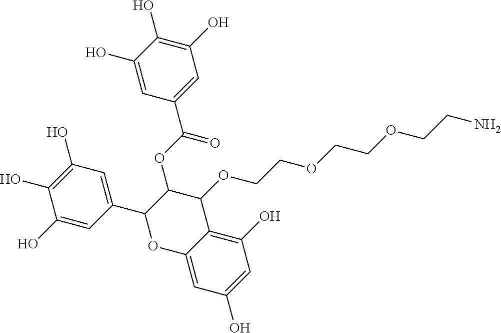 [SMILES: NCCOCCOCCOC1c2c(O)cc(O)cc2OC(c2cc(O)c(O)c(O)c2)C1OC(=O)c1cc(O)c(O)c(O)c1] | 55 |
| 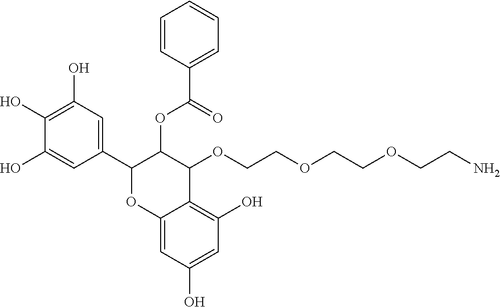 [SMILES: NCCOCCOCCOC1c2c(O)cc(O)cc2OC(c2cc(O)c(O)c(O)c2)C1OC(=O)c1ccccc1] | 56 |
| 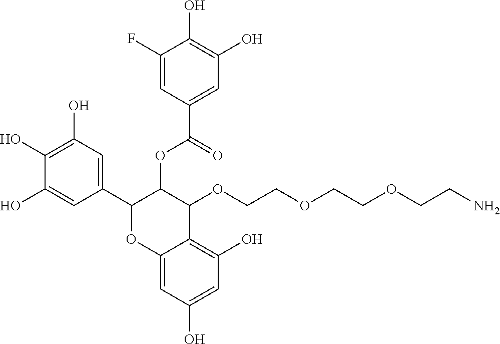 [SMILES: NCCOCCOCCOC1c2c(O)cc(O)cc2OC(c2cc(O)c(O)c(O)c2)C1OC(=O)c1cc(O)c(O)c(F)c1] | 57 |
| 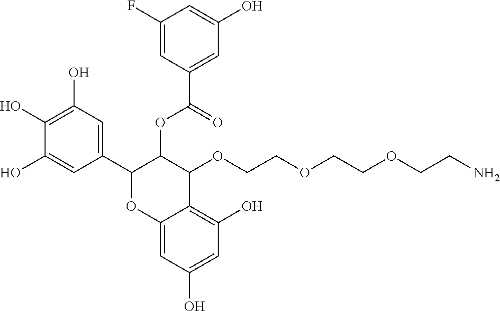 [SMILES: NCCOCCOCCOC1c2c(O)cc(O)cc2OC(c2cc(O)c(O)c(O)c2)C1OC(=O)c1cc(O)cc(F)c1] | 58 |
| 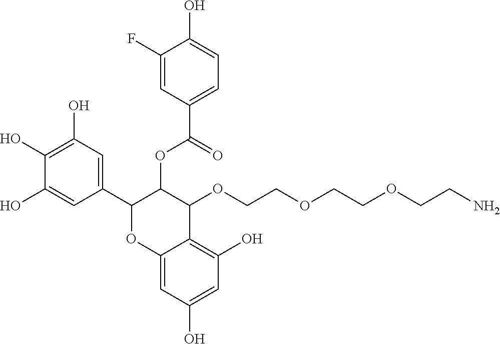 [SMILES: NCCOCCOCCOC1c2c(O)cc(O)cc2OC(c2cc(O)c(O)c(O)c2)C1OC(=O)c1ccc(O)c(F)c1] | 59 |
| 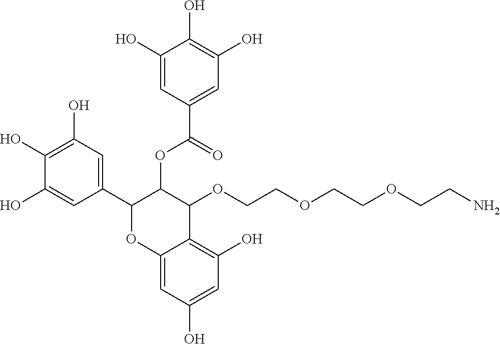 [SMILES: NCCOCCOCCOC1c2c(O)cc(O)cc2OC(c2cc(O)c(O)c(O)c2)C1OC(=O)c1cc(O)c(O)c(O)c1] | 60 |
| 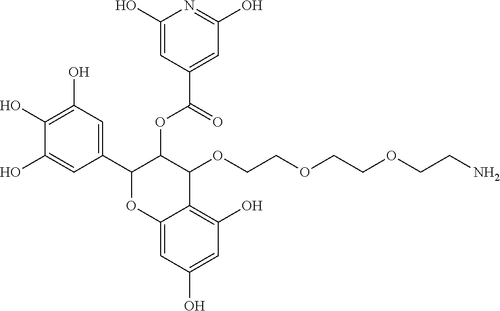 [SMILES: NCCOCCOCCOC1c2c(O)cc(O)cc2OC(c2cc(O)c(O)c(O)c2)C1OC(=O)c1cc(O)nc(O)c1] | 61 |
| 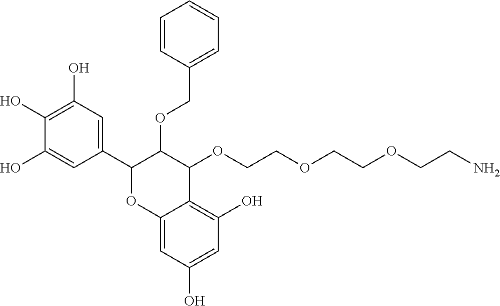 [SMILES: NCCOCCOCCOC1c2c(O)cc(O)cc2OC(c2cc(O)c(O)c(O)c2)C1OCc1ccccc1] | 62 |

TABLE E
Tetrameric analogs

| | |
|---|---|
| 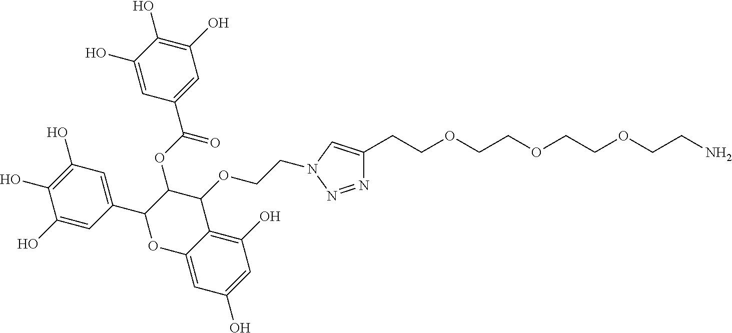 [SMILES: NCCOCCOCCOCCc1cn(CCOC2c3c(O)cc(O)cc3OC(c3cc(O)c(O)c(O)c3)C2OC(=O)c2cc(O)c(O)c(O)c2)nn1] | 63 |
| 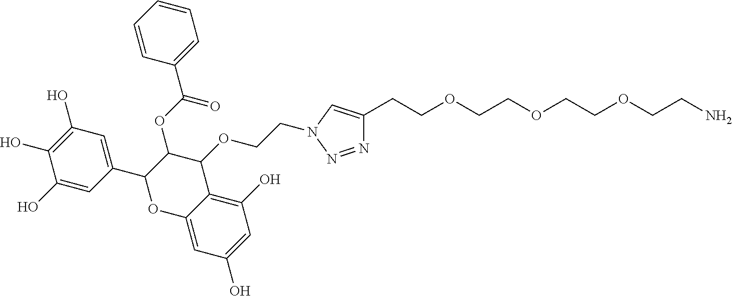 [SMILES: NCCOCCOCCOCCc1cn(CCOC2c3c(O)cc(O)cc3OC(c3cc(O)c(O)c(O)c3)C2OC(=O)c2ccccc2)nn1] | 64 |
| 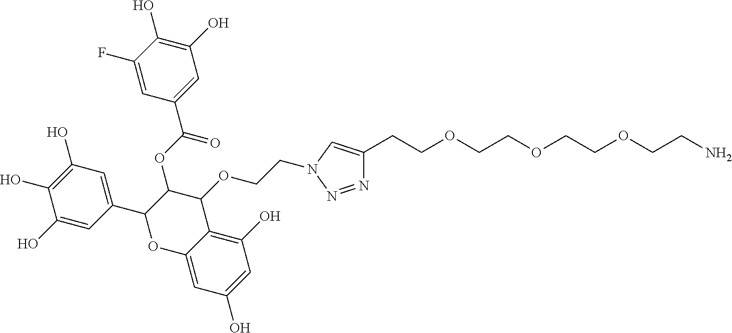 [SMILES: NCCOCCOCCOCCc1cn(CCOC2c3c(O)cc(O)cc3OC(c3cc(O)c(O)c(O)c3)C2OC(=O)c2cc(O)c(O)c(F)c2)nn1] | 65 |
| 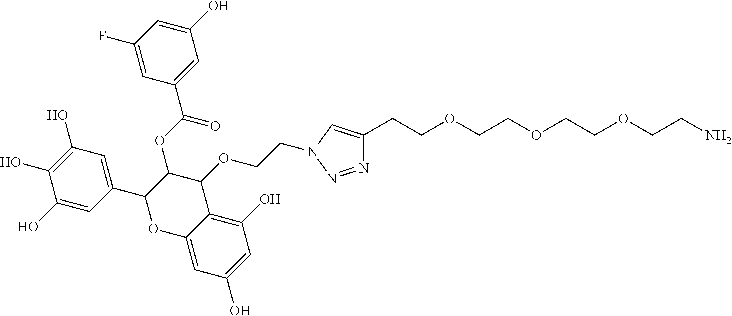 [SMILES: NCCOCCOCCOCCc1cn(CCOC2c3c(O)cc(O)cc3OC(c3cc(O)c(O)c(O)c3)C2OC(=O)c2cc(O)cc(F)c2)nn1] | 66 |
| 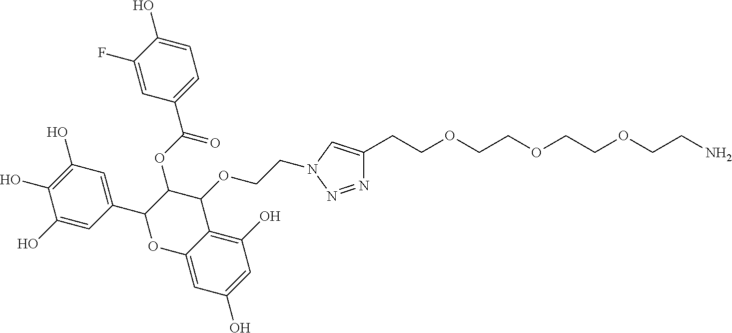 [SMILES: NCCOCCOCCOCCc1cn(CCOC2c3c(O)cc(O)cc3OC(c3cc(O)c(O)c(O)c3)C2OC(=O)c2ccc(O)c(F)c2)nn1] | 67 |
| 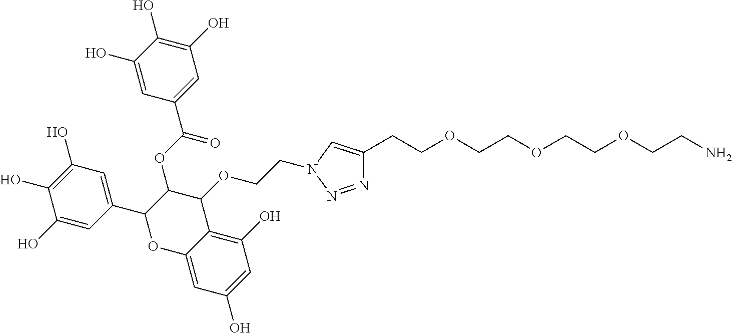 [SMILES: NCCOCCOCCOCCc1cn(CCOC2c3c(O)cc(O)cc3OC(c3cc(O)c(O)c(O)c3)C2OC(=O)c2cc(O)c(O)c(O)c2)nn1] | 68 |
| 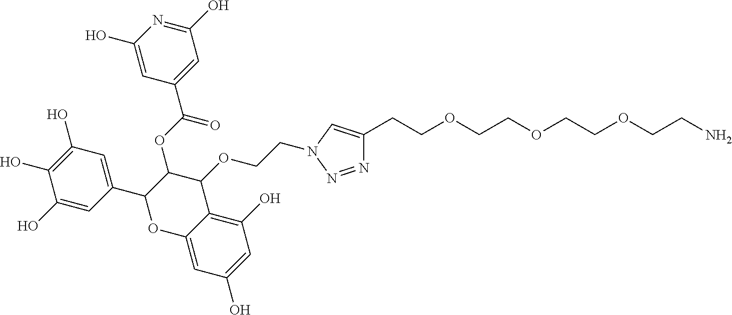 [SMILES: NCCOCCOCCOCCc1cn(CCOC2c3c(O)cc(O)cc3OC(c3cc(O)c(O)c(O)c3)C2OC(=O)c2cc(O)nc(O)c2)nn1] | 69 |
| 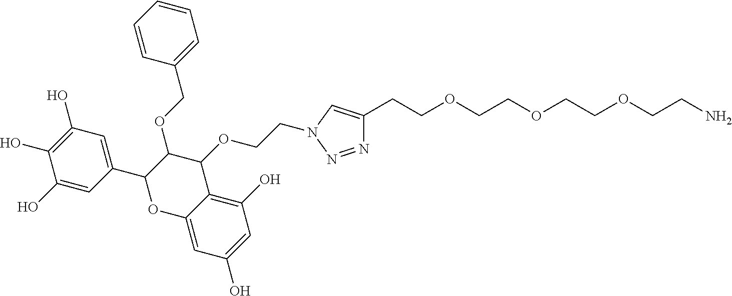 [SMILES: NCCOCCOCCOCCc1cn(CCOC2c3c(O)cc(O)cc3OC(c3cc(O)c(O)c(O)c3)C2OCc2ccccc2)nn1] | 70 |
| 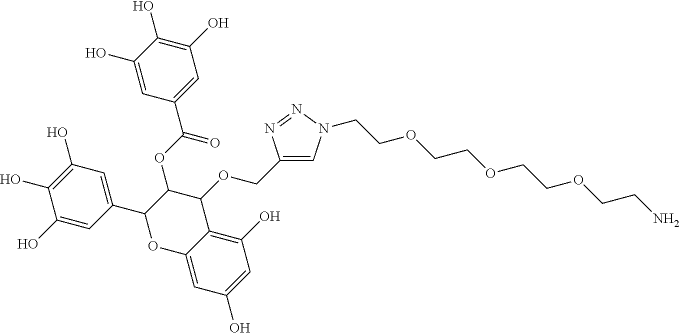 [SMILES: NCCOCCOCCOCCn1cc(COC2c3c(O)cc(O)cc3OC(c3cc(O)c(O)c(O)c3)C2OC(=O)c2cc(O)c(O)c(O)c2)nn1] | 71 |
| 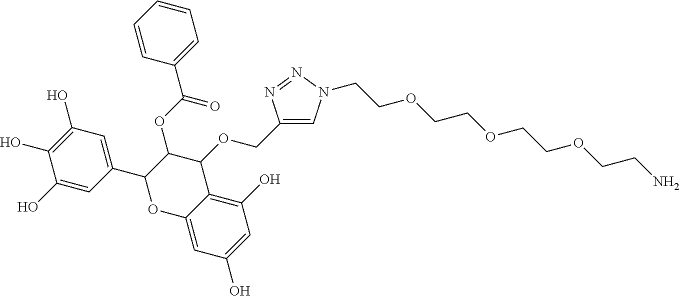 [SMILES: NCCOCCOCCOCCn1cc(COC2c3c(O)cc(O)cc3OC(c3cc(O)c(O)c(O)c3)C2OC(=O)c2ccccc2)nn1] | 72 |
| 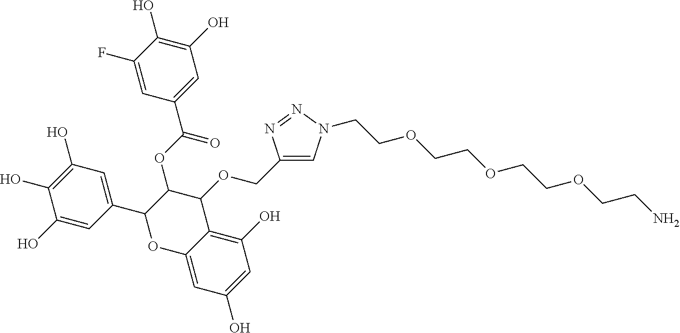 [SMILES: NCCOCCOCCOCCn1cc(COC2c3c(O)cc(O)cc3OC(c3cc(O)c(O)c(O)c3)C2OC(=O)c2cc(O)c(O)c(F)c2)nn1] | 73 |
| 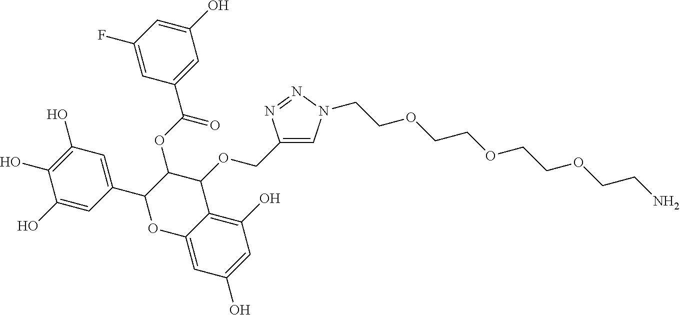 [SMILES: NCCOCCOCCOCCn1cc(COC2c3c(O)cc(O)cc3OC(c3cc(O)c(O)c(O)c3)C2OC(=O)c2cc(O)cc(F)c2)nn1] | 74 |
| 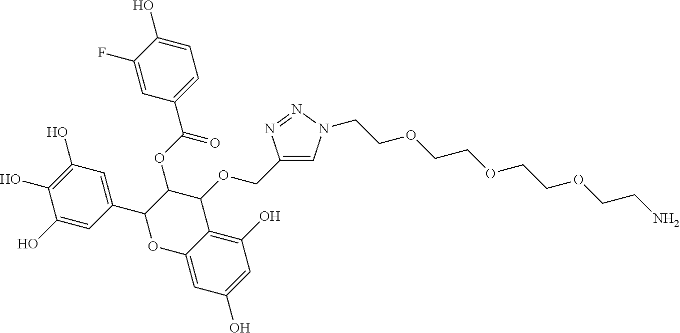 [SMILES: NCCOCCOCCOCCn1cc(COC2c3c(O)cc(O)cc3OC(c3cc(O)c(O)c(O)c3)C2OC(=O)c2ccc(O)c(F)c2)nn1] | 75 |
| 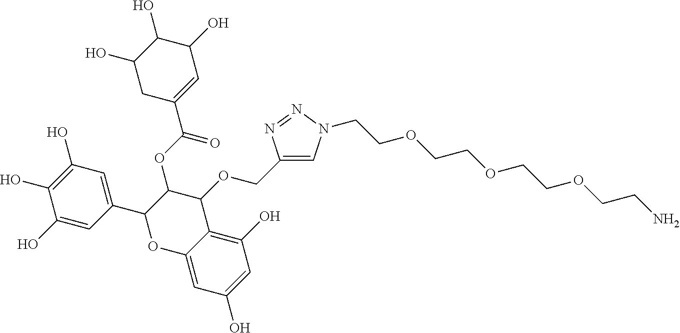 [SMILES: NCCOCCOCCOCCn1cc(COC2c3c(O)cc(O)cc3OC(c3cc(O)c(O)c(O)c3)C2OC(=O)C2=CC(O)C(O)C(O)C2)nn1] | 76 |

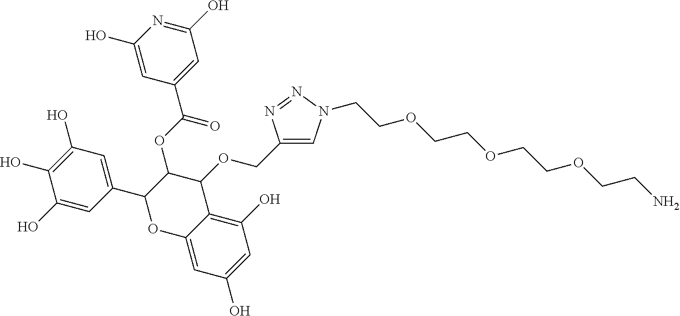 [SMILES: NCCOCCOCCOCCn1cc(COC2c3c(O)cc(O)cc3OC(c3cc(O)c(O)c(O)c3)C2OC(=O)c2cc(O)nc(O)c2)nn1]

| | |
|---|---|
| 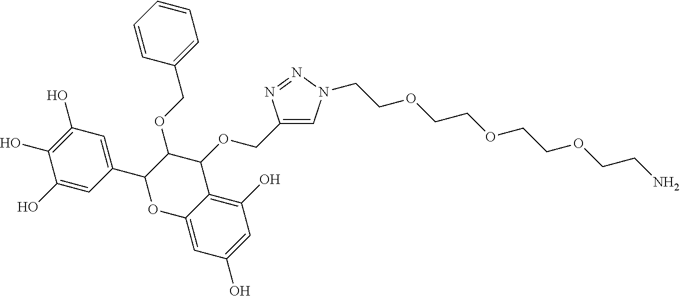 [SMILES: NCCOCCOCCOCCn1cc(COC2c3c(O)cc(O)cc3OC(c3cc(O)c(O)c(O)c3)C2OCc2ccccc2)nn1] | 78 |
| 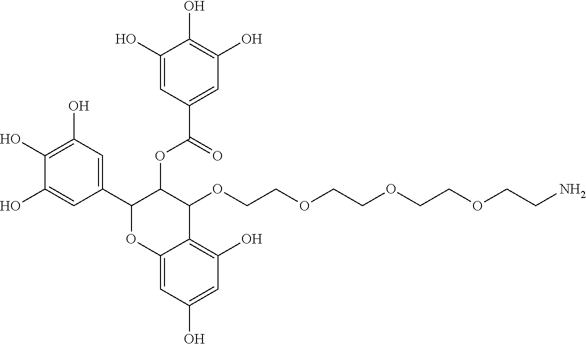 [SMILES: NCCOCCOCCOCCOC1c2c(O)cc(O)cc2OC(c2cc(O)c(O)c(O)c2)C1OC(=O)c1cc(O)c(O)c(O)c1] | 79 |
| 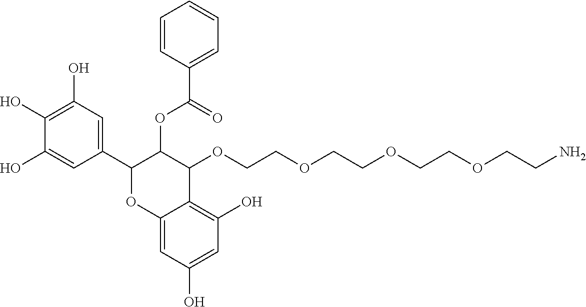 [SMILES: NCCOCCOCCOCCOC1c2c(O)cc(O)cc2OC(c2cc(O)c(O)c(O)c2)C1OC(=O)c1ccccc1] | 80 |
| 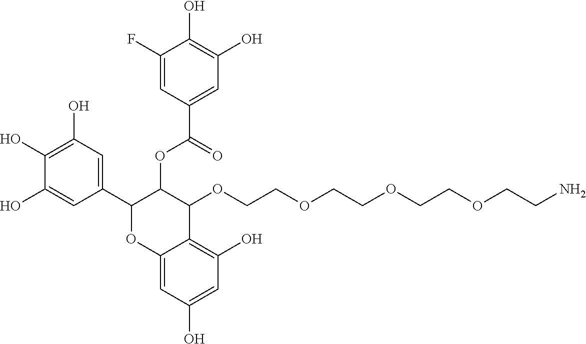 [SMILES: NCCOCCOCCOCCOC1c2c(O)cc(O)cc2OC(c2cc(O)c(O)c(O)c2)C1OC(=O)c1cc(O)c(O)c(F)c1] | 81 |
| 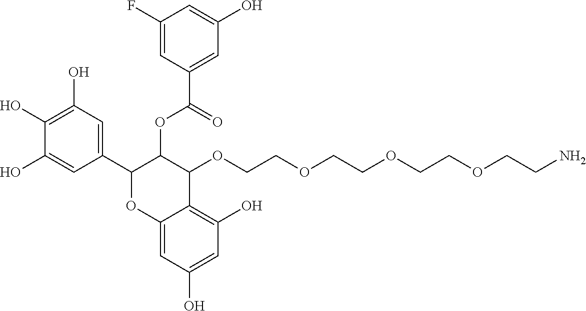 [SMILES: NCCOCCOCCOCCOC1c2c(O)cc(O)cc2OC(c2cc(O)c(O)c(O)c2)C1OC(=O)c1cc(O)cc(F)c1] | 82 |
| 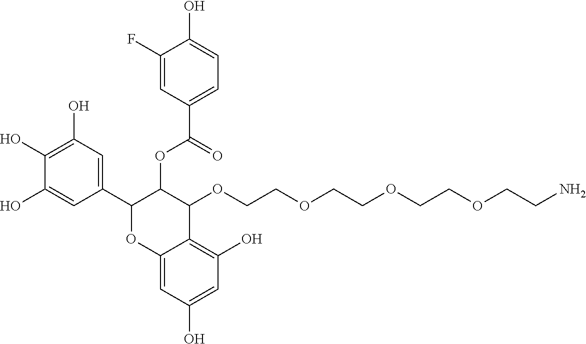 [SMILES: NCCOCCOCCOCCOC1c2c(O)cc(O)cc2OC(c2cc(O)c(O)c(O)c2)C1OC(=O)c1ccc(O)c(F)c1] | 83 |
| 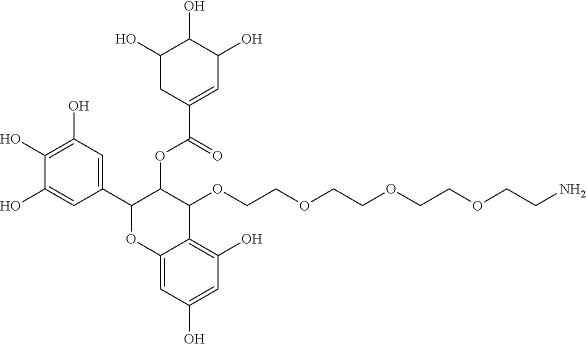 [SMILES: NCCOCCOCCOCCOC1c2c(O)cc(O)cc2OC(c2cc(O)c(O)c(O)c2)C1OC(=O)C1=CC(O)C(O)C(O)C1] | 84 |
| 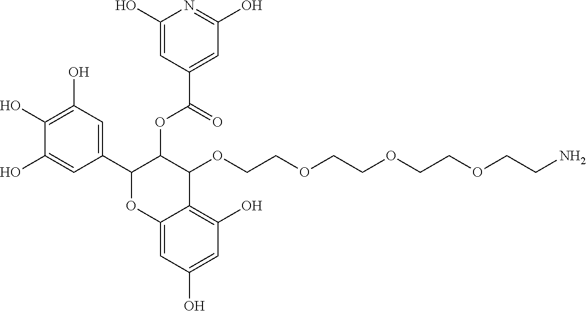 [SMILES: NCCOCCOCCOCCOC1c2c(O)cc(O)cc2OC(c2cc(O)c(O)c(O)c2)C1OC(=O)c1cc(O)nc(O)c1] | 85 |
| 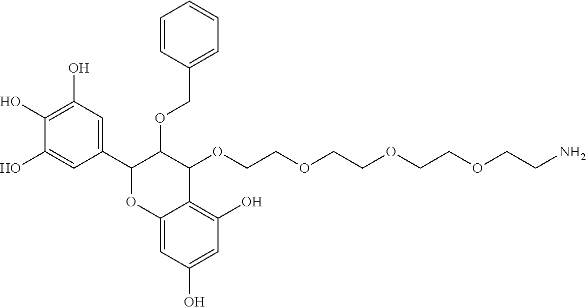 [SMILES: NCCOCCOCCOCCOC1c2c(O)cc(O)cc2OC(c2cc(O)c(O)c(O)c2)C1OCc1ccccc1] | 86 |

TABLE F
Pentameric analogs

| | |
|---|---|
| 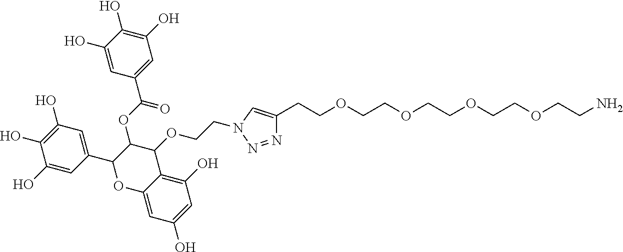 [SMILES: NCCOCCOCCOCCOCCc1cn(CCOC2c3c(O)cc(O)cc3OC(c3cc(O)c(O)c(O)c3)C2OC(=O)c2cc(O)c(O)c(O)c2)nn1] | 87 |
| 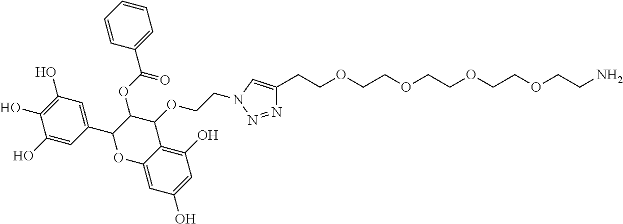 [SMILES: NCCOCCOCCOCCOCCc1cn(CCOC2c3c(O)cc(O)cc3OC(c3cc(O)c(O)c(O)c3)C2OC(=O)c2ccccc2)nn1] | 88 |
| 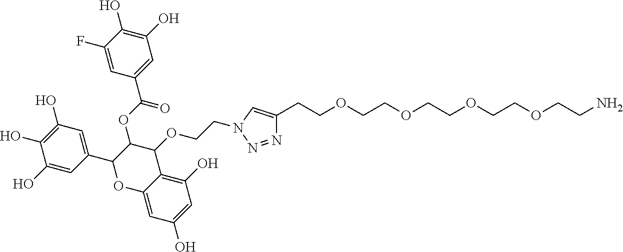 [SMILES: NCCOCCOCCOCCOCCc1cn(CCOC2c3c(O)cc(O)cc3OC(c3cc(O)c(O)c(O)c3)C2OC(=O)c2cc(O)c(O)c(F)c2)nn1] | 89 |
| 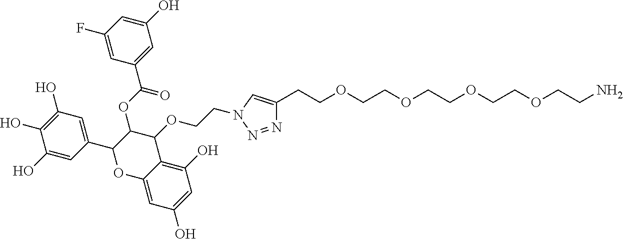 [SMILES: NCCOCCOCCOCCOCCc1cn(CCOC2c3c(O)cc(O)cc3OC(c3cc(O)c(O)c(O)c3)C2OC(=O)c2cc(O)cc(F)c2)nn1] | 90 |
| 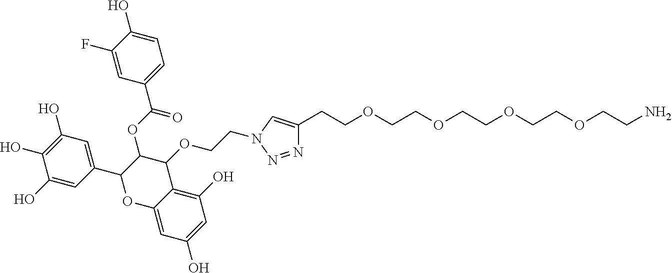 [SMILES: NCCOCCOCCOCCOCCc1cn(CCOC2c3c(O)cc(O)cc3OC(c3cc(O)c(O)c(O)c3)C2OC(=O)c2ccc(O)c(F)c2)nn1] | 91 |
| 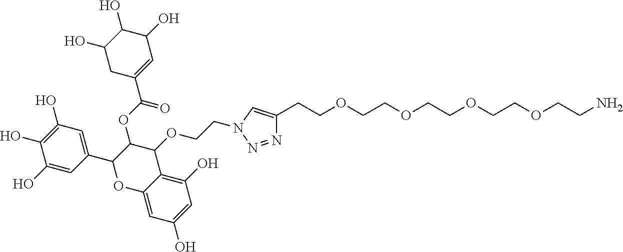 [SMILES: NCCOCCOCCOCCOCCc1cn(CCOC2c3c(O)cc(O)cc3OC(c3cc(O)c(O)c(O)c3)C2OC(=O)C2=CC(O)C(O)C(O)C2)nn1] | 92 |
| 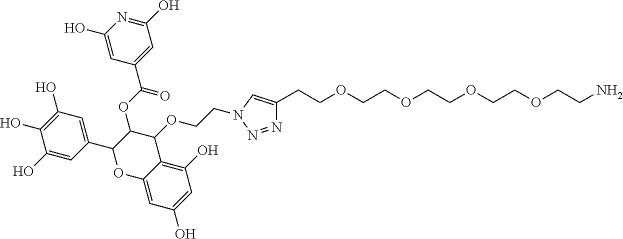 [SMILES: NCCOCCOCCOCCOCCc1cn(CCOC2c3c(O)cc(O)cc3OC(c3cc(O)c(O)c(O)c3)C2OC(=O)c2cc(O)nc(O)c2)nn1] | 93 |
| 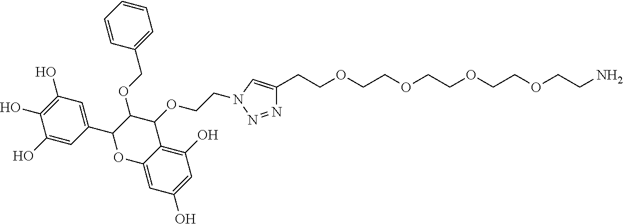 [SMILES: NCCOCCOCCOCCOCCc1cn(CCOC2c3c(O)cc(O)cc3OC(c3cc(O)c(O)c(O)c3)C2OCc2ccccc2)nn1] | 94 |
| 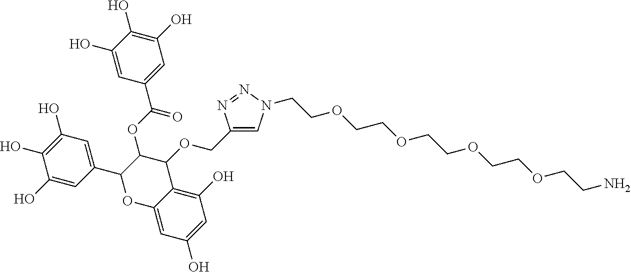 [SMILES: NCCOCCOCCOCCOCCn1cc(COC2c3c(O)cc(O)cc3OC(c3cc(O)c(O)c(O)c3)C2OC(=O)c2cc(O)c(O)c(O)c2)nn1] | 95 |
| 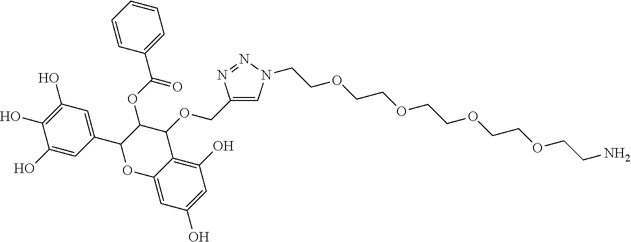 [SMILES: NCCOCCOCCOCCOCCn1cc(COC2c3c(O)cc(O)cc3OC(c3cc(O)c(O)c(O)c3)C2OC(=O)c2ccccc2)nn1] | 96 |
| 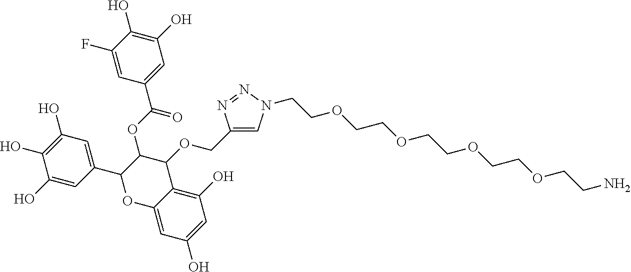 [SMILES: NCCOCCOCCOCCOCCn1cc(COC2c3c(O)cc(O)cc3OC(c3cc(O)c(O)c(O)c3)C2OC(=O)c2cc(O)c(O)c(F)c2)nn1] | 97 |
| 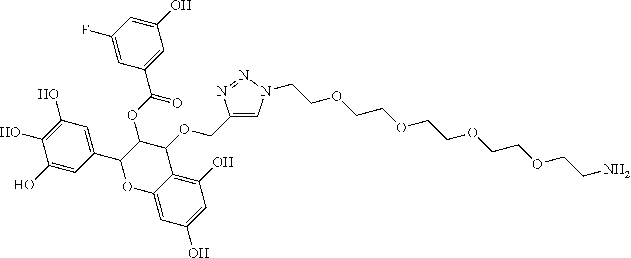 [SMILES: NCCOCCOCCOCCOCCn1cc(COC2c3c(O)cc(O)cc3OC(c3cc(O)c(O)c(O)c3)C2OC(=O)c2cc(O)cc(F)c2)nn1] | 98 |
| 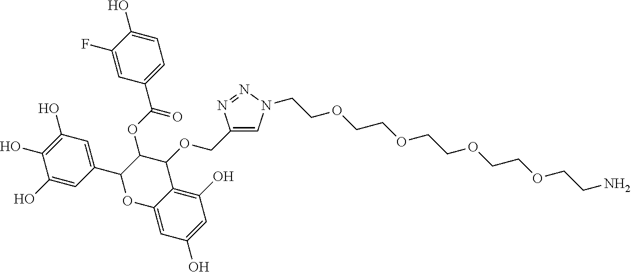 [SMILES: NCCOCCOCCOCCOCCn1cc(COC2c3c(O)cc(O)cc3OC(c3cc(O)c(O)c(O)c3)C2OC(=O)c2ccc(O)c(F)c2)nn1] | 99 |
| 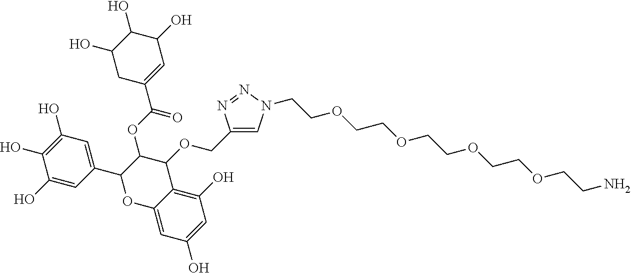 [SMILES: NCCOCCOCCOCCOCCn1cc(COC2c3c(O)cc(O)cc3OC(c3cc(O)c(O)c(O)c3)C2OC(=O)C2=CC(O)C(O)C(O)C2)nn1] | 100 |
| 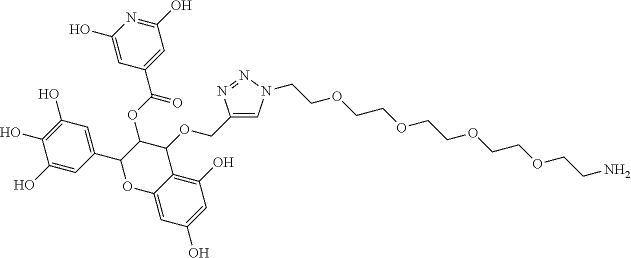 [SMILES: NCCOCCOCCOCCOCCn1cc(COC2c3c(O)cc(O)cc3OC(c3cc(O)c(O)c(O)c3)C2OC(=O)c2cc(O)nc(O)c2)nn1] | 101 |
| 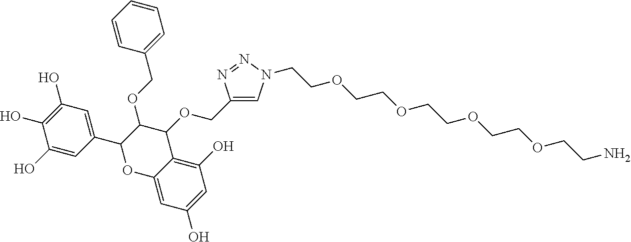 [SMILES: NCCOCCOCCOCCOCCn1cc(COC2c3c(O)cc(O)cc3OC(c3cc(O)c(O)c(O)c3)C2OCc2ccccc2)nn1] | 102 |
| 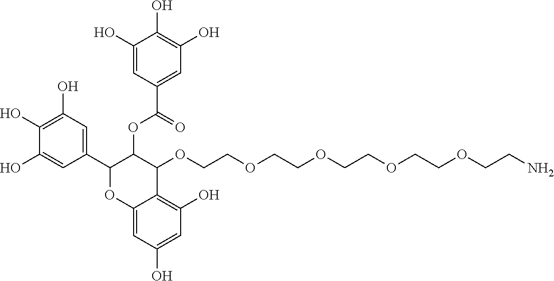 [SMILES: NCCOCCOCCOCCOCCOC1c2c(O)cc(O)cc2OC(c2cc(O)c(O)c(O)c2)C1OC(=O)c1cc(O)c(O)c(O)c1] | 103 |
| 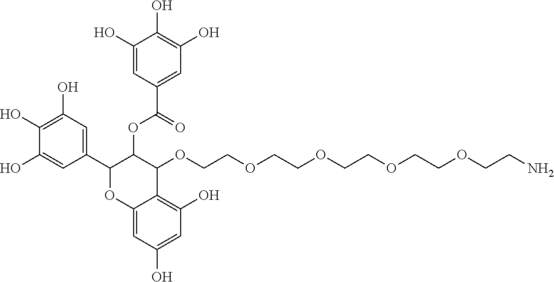 [SMILES: NCCOCCOCCOCCOCCOC1c2c(O)cc(O)cc2OC(c2cc(O)c(O)c(O)c2)C1OC(=O)c1cc(O)c(O)c(O)c1] | 104 |
| 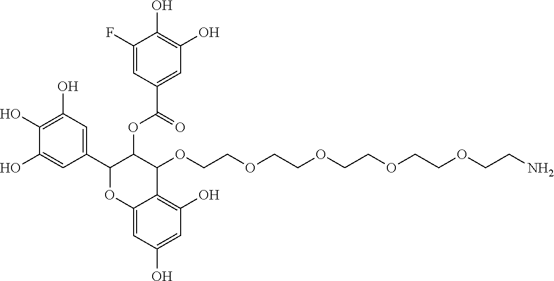 [SMILES: NCCOCCOCCOCCOCCOC1c2c(O)cc(O)cc2OC(c2cc(O)c(O)c(O)c2)C1OC(=O)c1cc(O)c(O)c(F)c1] | 105 |
| 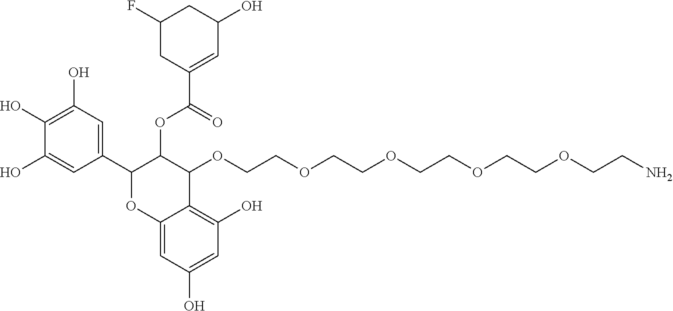 [SMILES: NCCOCCOCCOCCOCCOC1c2c(O)cc(O)cc2OC(c2cc(O)c(O)c(O)c2)C1OC(=O)C1=CC(O)CC(F)C1] | 106 |
| 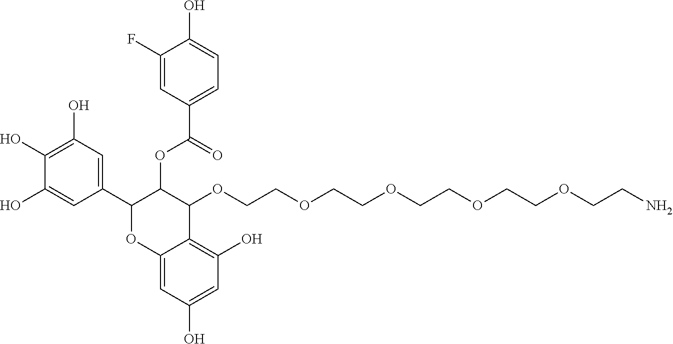 [SMILES: NCCOCCOCCOCCOCCOC1c2c(O)cc(O)cc2OC(c2cc(O)c(O)c(O)c2)C1OC(=O)c1ccc(O)c(F)c1] | 107 |
| 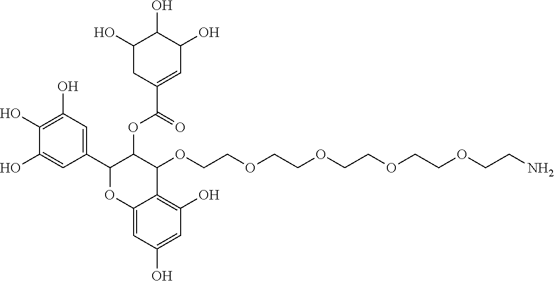 [SMILES: NCCOCCOCCOCCOCCOC1c2c(O)cc(O)cc2OC(c2cc(O)c(O)c(O)c2)C1OC(=O)C1=CC(O)C(O)C(O)C1] | 108 |
| 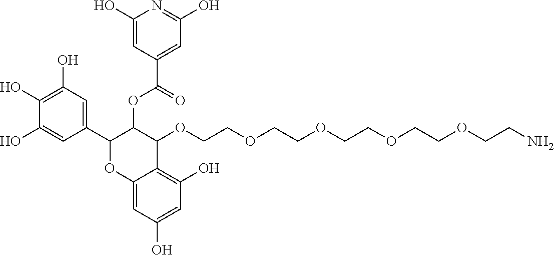 [SMILES: NCCOCCOCCOCCOCCOC1c2c(O)cc(O)cc2OC(c2cc(O)c(O)c(O)c2)C1OC(=O)c1cc(O)nc(O)c1] | 109 |
| 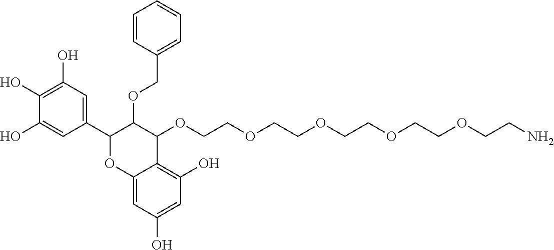 [SMILES: NCCOCCOCCOCCOCCOC1c2c(O)cc(O)cc2OC(c2cc(O)c(O)c(O)c2)C1OCc1ccccc1] | 110 |

TABLE G
Hexameric analogs

| | |
|---|---|
| 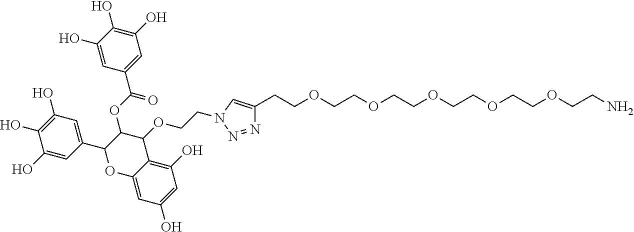 [SMILES: NCCOCCOCCOCCOCCOCCc1cn(CCOC2c3c(O)cc(O)cc3OC(c3cc(O)c(O)c(O)c3)C2OC(=O)c2cc(O)c(O)c(O)c2)nn1] | 111 |
| 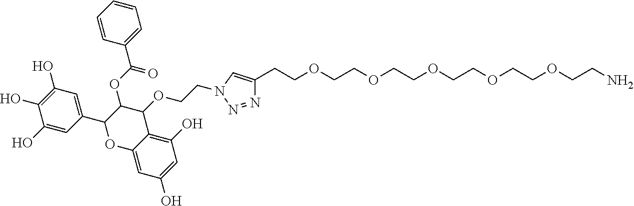 [SMILES: NCCOCCOCCOCCOCCOCCc1cn(CCOC2c3c(O)cc(O)cc3OC(c3cc(O)c(O)c(O)c3)C2OC(=O)c2ccccc2)nn1] | 112 |
| 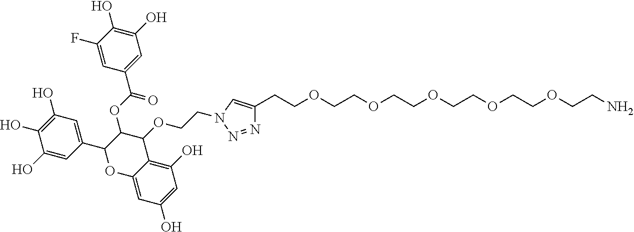 [SMILES: NCCOCCOCCOCCOCCOCCc1cn(CCOC2c3c(O)cc(O)cc3OC(c3cc(O)c(O)c(O)c3)C2OC(=O)c2cc(O)c(O)c(F)c2)nn1] | 113 |
| 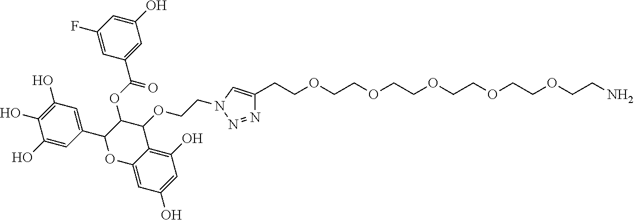 [SMILES: NCCOCCOCCOCCOCCOCCc1cn(CCOC2c3c(O)cc(O)cc3OC(c3cc(O)c(O)c(O)c3)C2OC(=O)c2cc(O)cc(F)c2)nn1] | 114 |
| 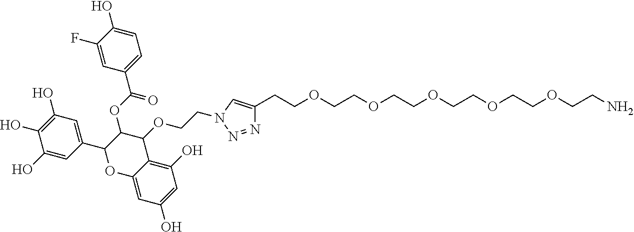 [SMILES: NCCOCCOCCOCCOCCOCCc1cn(CCOC2c3c(O)cc(O)cc3OC(c3cc(O)c(O)c(O)c3)C2OC(=O)c2ccc(O)c(F)c2)nn1] | 115 |
| 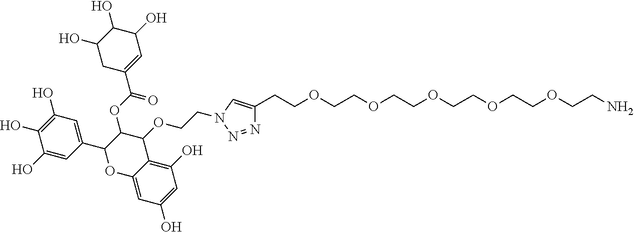 [SMILES: NCCOCCOCCOCCOCCOCCc1cn(CCOC2c3c(O)cc(O)cc3OC(c3cc(O)c(O)c(O)c3)C2OC(=O)C2=CC(O)C(O)C(O)C2)nn1] | 116 |
| 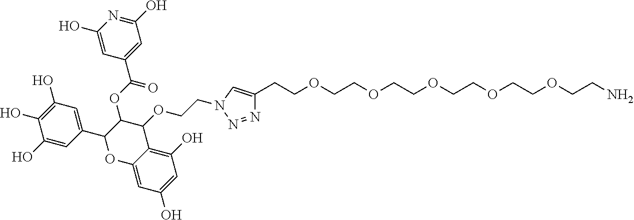 [SMILES: NCCOCCOCCOCCOCCOCCc1cn(CCOC2c3c(O)cc(O)cc3OC(c3cc(O)c(O)c(O)c3)C2OC(=O)c2cc(O)nc(O)c2)nn1] | 117 |
| 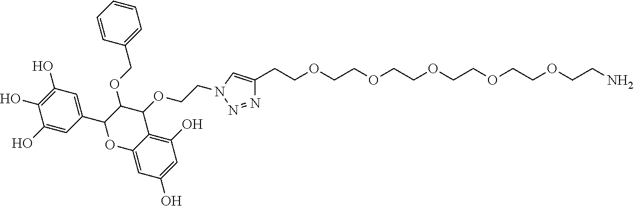 [SMILES: NCCOCCOCCOCCOCCOCCc1cn(CCOC2c3c(O)cc(O)cc3OC(c3cc(O)c(O)c(O)c3)C2OCc2ccccc2)nn1] | 118 |
| 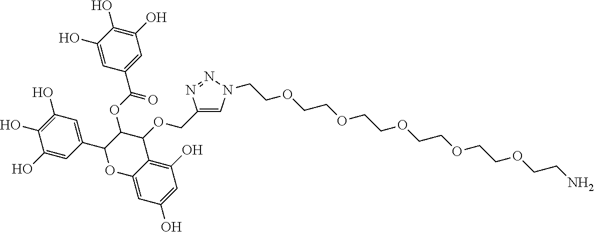 [SMILES: NCCOCCOCCOCCOCCOCCn1cc(COC2c3c(O)cc(O)cc3OC(c3cc(O)c(O)c(O)c3)C2OC(=O)c2cc(O)c(O)c(O)c2)nn1] | 119 |
| 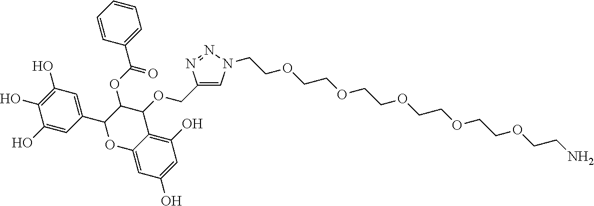 [SMILES: NCCOCCOCCOCCOCCOCCn1cc(COC2c3c(O)cc(O)cc3OC(c3cc(O)c(O)c(O)c3)C2OC(=O)c2ccccc2)nn1] | 120 |
| 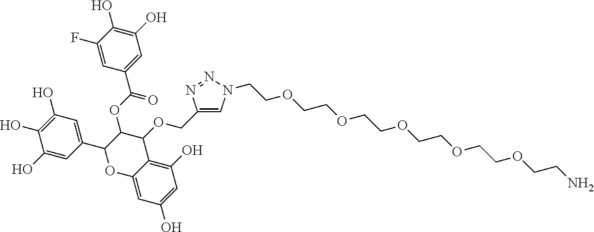 [SMILES: NCCOCCOCCOCCOCCOCCn1cc(COC2c3c(O)cc(O)cc3OC(c3cc(O)c(O)c(O)c3)C2OC(=O)c2cc(O)c(O)c(F)c2)nn1] | 121 |
| 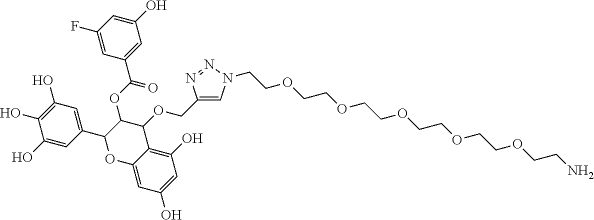 [SMILES: NCCOCCOCCOCCOCCOCCn1cc(COC2c3c(O)cc(O)cc3OC(c3cc(O)c(O)c(O)c3)C2OC(=O)c2cc(O)cc(F)c2)nn1] | 122 |
| 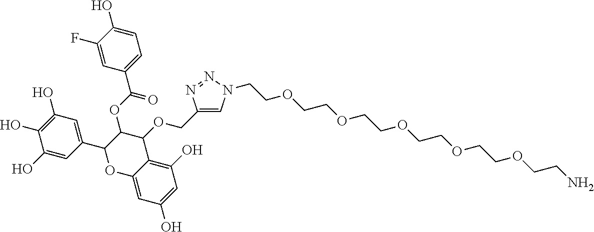 [SMILES: NCCOCCOCCOCCOCCOCCn1cc(COC2c3c(O)cc(O)cc3OC(c3cc(O)c(O)c(O)c3)C2OC(=O)c2ccc(O)c(F)c2)nn1] | 123 |
| 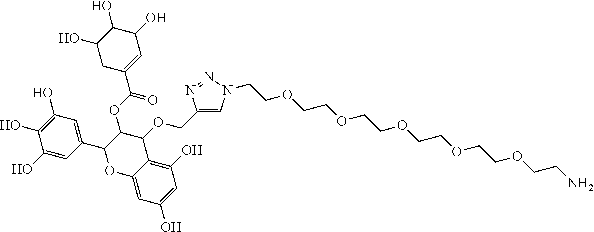 [SMILES: NCCOCCOCCOCCOCCOCCn1cc(COC2c3c(O)cc(O)cc3OC(c3cc(O)c(O)c(O)c3)C2OC(=O)C2=CC(O)C(O)C(O)C2)nn1] | 124 |
| 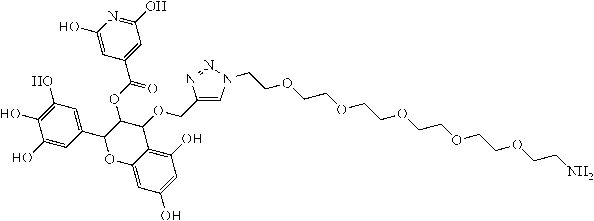 [SMILES: NCCOCCOCCOCCOCCOCCn1cc(COC2c3c(O)cc(O)cc3OC(c3cc(O)c(O)c(O)c3)C2OC(=O)c2cc(O)nc(O)c2)nn1] | 125 |
| 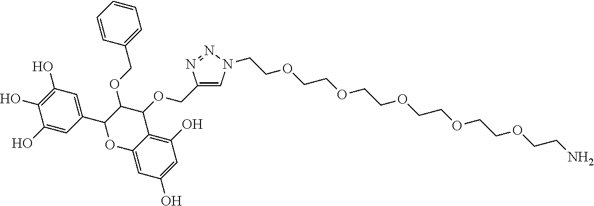 [SMILES: NCCOCCOCCOCCOCCOCCn1cc(COC2c3c(O)cc(O)cc3OC(c3cc(O)c(O)c(O)c3)C2OCc2ccccc2)nn1] | 126 |
| 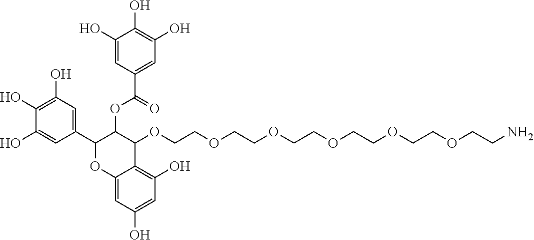 [SMILES: NCCOCCOCCOCCOCCOCCOC1c2c(O)cc(O)cc2OC(c2cc(O)c(O)c(O)c2)C1OC(=O)c1cc(O)c(O)c(O)c1] | 127 |
| 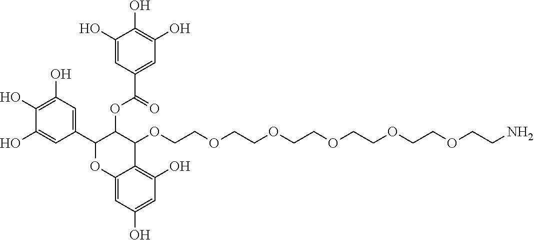 [SMILES: NCCOCCOCCOCCOCCOCCOC1c2c(O)cc(O)cc2OC(c2cc(O)c(O)c(O)c2)C1OC(=O)c1cc(O)c(O)c(O)c1] | 128 |
| 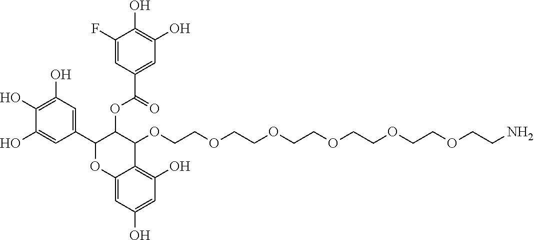 [SMILES: NCCOCCOCCOCCOCCOCCOC1c2c(O)cc(O)cc2OC(c2cc(O)c(O)c(O)c2)C1OC(=O)c1cc(O)c(O)c(F)c1] | 129 |
| 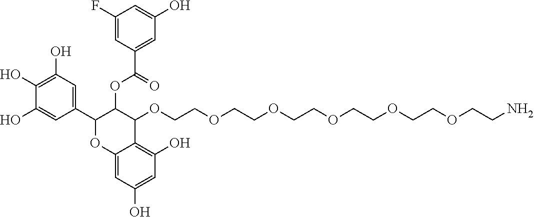 [SMILES: NCCOCCOCCOCCOCCOCCOC1c2c(O)cc(O)cc2OC(c2cc(O)c(O)c(O)c2)C1OC(=O)c1cc(O)cc(F)c1] | 130 |
| 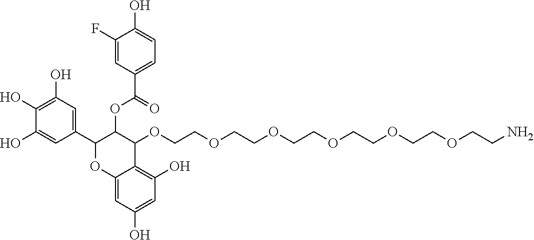 [SMILES: NCCOCCOCCOCCOCCOCCOC1c2c(O)cc(O)cc2OC(c2cc(O)c(O)c(O)c2)C1OC(=O)c1ccc(O)c(F)c1] | 131 |
| 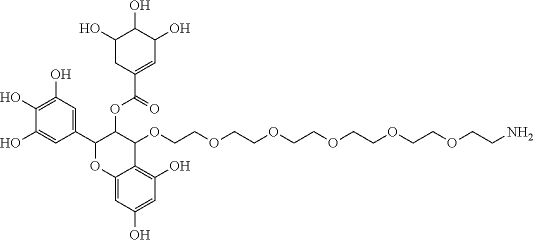 [SMILES: NCCOCCOCCOCCOCCOCCOC1c2c(O)cc(O)cc2OC(c2cc(O)c(O)c(O)c2)C1OC(=O)C1=CC(O)C(O)C(O)C1] | 132 |
| 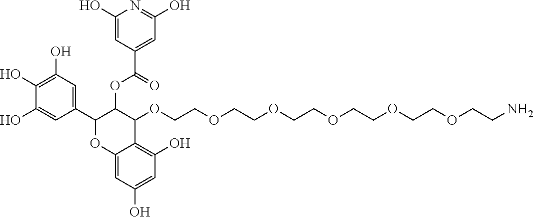 [SMILES: NCCOCCOCCOCCOCCOCCOC1c2c(O)cc(O)cc2OC(c2cc(O)c(O)c(O)c2)C1OC(=O)c1cc(O)nc(O)c1] | 133 |
| 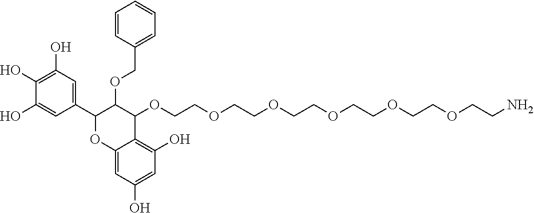 [SMILES: NCCOCCOCCOCCOCCOCCOC1c2c(O)cc(O)cc2OC(c2cc(O)c(O)c(O)c2)C1OCc1ccccc1] | 134 |

TABLE H
D ring derivatization

| | |
|---|---|
| 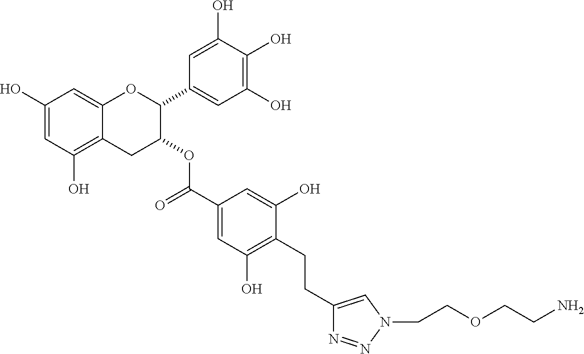 [SMILES: NCCOCCn1cc(CCc2c(O)cc(C(=O)O[C@@H]3Cc4c(O)cc(O)cc4O[C@@H]3c3cc(O)c(O)c(O)c3)cc2O)nn1] | 10 |
| 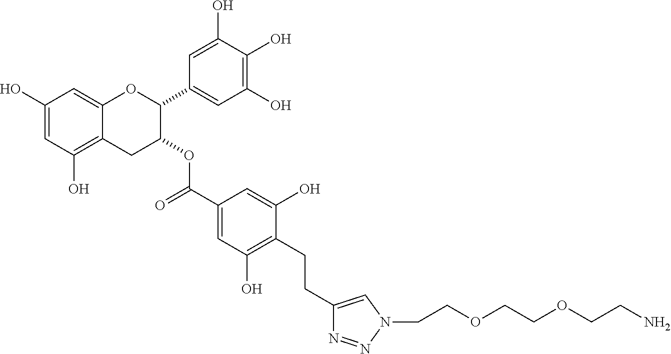 [SMILES: NCCOCCOCCn1cc(CCc2c(O)cc(C(=O)O[C@@H]3Cc4c(O)cc(O)cc4O[C@@H]3c3cc(O)c(O)c(O)c3)cc2O)nn1] | 11 |
| 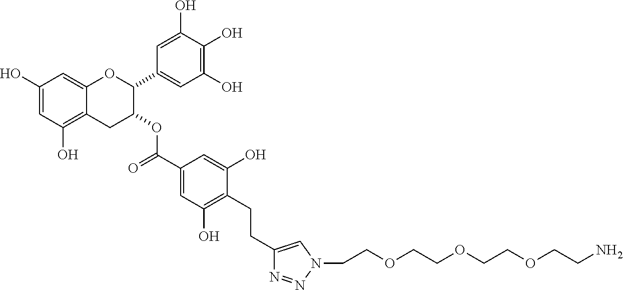 [SMILES: NCCOCCOCCOCCn1cc(CCc2c(O)cc(C(=O)O[C@@H]3Cc4c(O)cc(O)cc4O[C@@H]3c3cc(O)c(O)c(O)c3)cc2O)nn1] | 12 |
| 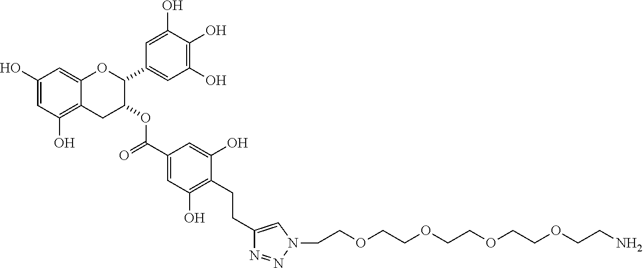 [SMILES: NCCOCCOCCOCCOCCn1cc(CCc2c(O)cc(C(=O)O[C@@H]3Cc4c(O)cc(O)cc4O[C@@H]3c3cc(O)c(O)c(O)c3)cc2O)nn1] | 13 |
| 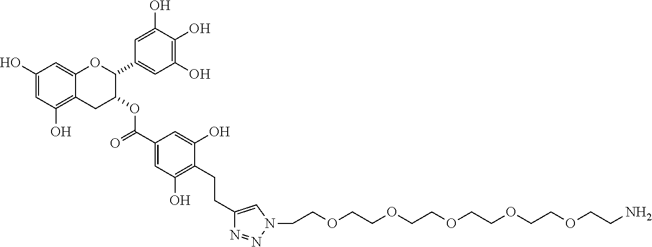 [SMILES: NCCOCCOCCOCCOCCOCCn1cc(CCc2c(O)cc(C(=O)O[C@@H]3Cc4c(O)cc(O)cc4O[C@@H]3c3cc(O)c(O)c(O)c3)cc2O)nn1] | 14 |

### INCORPORATION BY REFERENCE

Each publication and patent mentioned herein is hereby incorporated by reference in its entirety. In case of conflict, the present specification, including any definitions herein, will control.

### EQUIVALENTS

While specific embodiments of the subject invention have been discussed, the above specification is illustrative and not restrictive. Many variations of the invention will become apparent to those skilled in the art upon review of the preceding description and the following claims. The full scope of the invention should be determined by reference to the claims, along with their full scope of equivalents, and by reference to the rest of the specification, along with such variations.

What is claimed is:

1. An agent comprising a flavanol or a flavanol analog, a linker coupled to the flavanol or the flavanol analog, and a carrier coupled to the linker.

2. The agent of claim 1, wherein the flavanol or the flavanol analog comprises a compound selected from Table J.

3. The agent of claim 2, wherein the linker comprises a compound selected from Table L.

4. The agent of any one of claims 1 to 3, wherein the carrier comprises a nanoparticle.

5. The agent of claim 4, wherein the nanoparticle is an iron oxide nanoparticle (IONP).

6. The agent of claim 5, wherein the IONP is selected from Table N.

7. The agent of any one of claims 1 to 6, wherein the carrier comprises a coating that comprises polyethylene glycol (PEG), dextran, starch, chitosan, lipid, citrate, polyaniline, meso-2,3-dimercaptosuccinic acid, poly(maleic anhydride-alt-1-octadecene), polyacrylamide, phosphonate, silica, or a protein or a peptide segment having a sequence that is recognized by membrane-embedded proteins in the brain endothelium for conveying the agent across the blood-brain barrier.

8. The agent of claim 7, wherein the coating comprises dextran, and wherein the dextran comprises dextran-20 kDa, dextran-40 kDa, carboxy dextran, or cross-linked dextran-20 kDa.

9. The agent of claim 7, wherein the coating comprises PEG, optionally wherein the carrier is coupled to the linker via said PEG.

10. The agent of any one of claims 1 to 9, wherein the nanoparticle has a hydrodynamic particle size that is at least 4 nanometers and at most 150 nanometers as measured by dynamic light scattering.

11. The agent of any one of claims 1 to 10, wherein the agent further comprises an anti-amyloid antibody coupled to the carrier, or alternatively coupling to the carrier a peptide segment that targets the agent to an amyloid protein of interest, and potentially interferes with aggregation of the said amyloid by capping the target fibril as an additional mode of amyloid inhibition.

12. The agent of any one of claims 1 to 11, wherein the agent destabilizes a tau amyloid fibril when in contact with said tau amyloid fibril.

13. The agent of any one of claims 1 to 12, wherein the agent is permeable across the blood-brain barrier.

14. A composition for treating a tauopathy, wherein the composition comprises the agent of any one of claims 1 to 13.

15. The composition of claim 14, wherein the composition is effective in treating Alzheimer's disease.

16. A method of preparing the agent of any one of claims 1 to 13, wherein the method comprises reacting the flavanol or the flavanol analog with propargyl bromide, thereby forming a propargylated compound.

17. The method of claim 16, further comprising reacting the propargylated compound with an amino azide and tris((1-benzyl-4-triazolyl)methyl)amine, thereby forming a conjugate of the flavanol or the flavanol analog with a linker.

18. The method of claim 17, further comprising reacting the conjugate with the carrier, thereby covalently attaching the two to each other, to form the agent.

19. A method of treating a tauopathy in a subject, comprising administering to the subject an agent of any one of claims 1 to 13 or the composition of claim 14 or 15.

20. The method of claim 19, wherein said administering is intravenous.

21. The method of claim 19 or 20, wherein said tauopathy comprises Alzheimer's disease.

22. The method of any one of claims 19 to 21, wherein said tauopathy comprises progressive supranuclear palsy or chronic traumatic encephalopathy or other tauopathy.

23. A method of destabilizing a tau amyloid fibril, comprising contacting the tau amyloid fibril with the agent of any one of claims 1 to 13.

24. The method of claim 23, wherein the tau amyloid fibril comprises a paired helical filament.

25. The method of claim 24, wherein the agent dis-aggregates the paired helical filament.

26. The method of claim 23, wherein the agent disrupts ion pairing of lysine at position 340 of tau with respect to SEQ ID NO: 1 by forming at least one hydrogen bond with said lysine.
